SEQUENCE LISTING (34 sequences)

SEQ ID NO: 1 - protein, 1368 aa, Artificial Sequence [sequence omitted]

SEQ ID NO: 2 - protein, 573 aa, Artificial Sequence
  N-split-Cas9-v1 (split between 573 and 574)
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIE

SEQ ID NO: 3 - protein, 637 aa, Artificial Sequence
  N-split-Cas9-v2 (split between 637 and 638)
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLK

SEQ ID NO: 4 - protein, 795 aa, Artificial Sequence
  C-split-Cas9-v1 (split between 573 and 574)
CFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 5 - protein, 731 aa, Artificial Sequence
  C-split-Cas9-v2 (split between 637 and 638)
TYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 6 - protein, 138 aa, Artificial Sequence
  Intein from N. punctiforme
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNMIKIATRKYLGKQNVYDIGVERDHNFALKNGFIASN

SEQ ID NO: 7 - protein, 145 aa, Artificial Sequence
  NrdJ-1 Intein
CLVGSSEIITRNYGKTTIKEVVEIFDNDKNIQVLAFNTHTDNIEWAPIKAAQLTRPNAELVELEIDTLHGVKTIRCTPDHPVYTKNRGYVRADELTDDDELVVAIMEAKTYIGKLKSRKIVSNEDTYDIQTSTHNFFANDILVHN

SEQ ID NO: 8 - protein, 125 aa, Artificial Sequence
  Gp41-1 Intein
CLDLKTQVQTPQGMKEISNIQVGDLVLSNTGYNEVLNVFPKSKKKSYKITLEDGKEIICSEEHLFPTQTGEMNISGGLKEGMCLYVKEMMLKKILKIEELDERELIDIEVSGNHLFYANDILTHN

SEQ ID NO: 9 - protein, 102 aa, Artificial Sequence
  N-terminal splicing region of N. punctiforme
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 10 - protein, 105 aa, Artificial Sequence
  N-terminal splicing region of NrdJ-1
CLVGSSEIITRNYGKTTIKEVVEIFDNDKNIQVLAFNTHTDNIEWAPIKAAQLTRPNAELVELEIDTLHGVKTIRCTPDHPVYTKNRGYVRADELTDDDELVVAI

SEQ ID NO: 11 - protein, 88 aa, Artificial Sequence
  N-terminal splicing region of gp41-1
CLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 12 - protein, 36 aa, Artificial Sequence
  C-terminal splicing region of N. punctiforme
MIKIATRKYLGKQNVYDIGVERDHNFALKNGFIASN

SEQ ID NO: 13 - protein, 40 aa, Artificial Sequence
  C-terminal splicing region of NrdJ-1
MEAKTYIGKLKSRKIVSNEDTYDIQTSTHNFFANDILVHN

SEQ ID NO: 14 - protein, 37 aa, Artificial Sequence
  C-terminal splicing region of g41-1
MMLKKILKIEELDERELIDIEVSGNHLFYANDILTHN

SEQ ID NO: 15 - protein, 7 aa, Artificial Sequence
  SV40 NLS
PKKKRKV

SEQ ID NO: 16 - protein, 17 aa, Artificial Sequence
  2xSV40 NLS
PKKKRKVEDPKKKRKVD

SEQ ID NO: 17 - protein, 20 aa, Artificial Sequence
  Nucleoplasmin NLS
AVKRPAATKKAGQAKKKKLD

SEQ ID NO: 18 - protein, 25 aa, Artificial Sequence
  MSRRRKANPTKLSENAKKLAKEVEN
MSRRRKANPTKLSENAKKLAKEVEN

SEQ ID NO: 19 - protein, 9 aa, Artificial Sequence
  c-Myc NLS
PAAKRVKLD

SEQ ID NO: 20 - protein, 9 aa, Artificial Sequence
  TUS protein NLS
KLKIKRPVK

SEQ ID NO: 21 - DNA, 257 bp, Artificial Sequence
  U6 promoter
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacg

SEQ ID NO: 22 - DNA, 799 bp, Artificial Sequence
  CHB promoter
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgg

SEQ ID NO: 23 - DNA, 1095 bp, Artificial Sequence
  Human exon 51 of DMD
aacaggattgtctaccagacattttaattctagtactatgcatcttaaccattaccataggctgacttactctacagtgtccaacactattcatattaagatttatttaatgactttgaaacagtatttcatgtctaaatagaaaaactactaactcgcatttttaagaaaatattgtatcttggtttttcttcactgctggccagtttactaacaatctgaaataaaaagaaaaaaatatgataaactgctcccagtataaaatacagagctaagacaagaacgtttcattggctttgatttccctagggtccagcttcaaattaatttacttcctattcaagggaattttaaatcagaaagaagatcttatcccatcttgttttgcctttgttttttcttgaataaaaaaaaaataagtaaaatttatttccctggcaaggtctgaaaacttttgttttctttaccacttccacaatgtatatgattgttactgagaaggcttatttaacttaagttacttgtccaggcatgagaatgagcaaaatcgttttttaaaaaattgttaaatgtatattaatgaaaaggttgaatcttttcattttctaccatgtattgctaaacaaagtatccacattgttagaaaaagatatataatgtcatgaataagagtttggctcaaattgttactcttcaattaaatttgacttattgttattgaaattggctctttagcttgtgtttctaatttttctttttcttcttttttcctttttgcaaaaacccaaaatattttagctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggtatgagaaaaaatgataaaagttggcagaagtttttctttaaaatgaagattttccaccaatc

SEQ ID NO: 24 - DNA, 1029 bp, Artificial Sequence
  Porcine exon 51 DMD
gagaggaattctctgccagctatttttactctggaacttcaggtcttaaccattaccttgtgttcccttacaccacaatgttcaacactattaacattaatttaatatttcttaagagccttgaaatggtgtctcattatccaagtggaaaaactttactagctctgattttccaagaataatatagcttgggttttctttactgctagccactttattatcagtctgaaagaaaaagaaaaaaaatatataaactgctaccaatataaaataaagagcaaaggcaagaacatttcattggttttgatttccctagagtccacttccaaagcaatttacctcttattcaaggaaattgtaagtttgaaataagatcttatccaatttttacttttttccccttgaacaaaaaaaaaagtaaaacattcaccttatcaaagtttgaatactttatatttttctttaccagttcagcaactgtataattgttattgagggctttttaggttaaattaattgctcacctataggaataatcaaaatggttttgaaagattgttaaatatgtattaatgaaaaatttgaatctttgaattattctaccatgtagtactcatgtttctaggagaaaaacatgtaatgtcataaaaaatactttggctcaaattgttgccctttaattagttttattgttattgaaactggcttcttagcttgtgtttctaattttccttttttctttttccctttttgaaaaaaaaaatttagctactagtcagactgttactctggtgacacaacccacagttaccaaggaaactgccatctccaaaccagaaatgccatcttccttgctgttggaggtacctgctctggcagatttcaacagggcttggacagaacttaccgactggctgtctctgcttgatcgagttataaaatcacagagggtgatggtgggtgatcttgaagacatcaacgagatgatcatcaagcagaaggtatggggggaataaagataagagctgg

SEQ ID NO: 25 - DNA, 21 bp, Artificial Sequence
  first spacer sequence pig
gagagttcctaaggtagagag

SEQ ID NO: 26 - DNA, 21 bp, Artificial Sequence
  first spacer sequence human
gataaagataagagctggcag

SEQ ID NO: 27 - DNA, 21 bp, Artificial Sequence
  second spacer sequence pig
gtaatttgaagctggacccta

SEQ ID NO: 28 - DNA, 20 bp, Artificial Sequence
  second spacer sequence human
gtctaggagagtaaagtgat

SEQ ID NO: 29 - DNA, 93 bp, Artificial Sequence
  sgRNA backbone sequence
gtttaagagctatgctggaaacagcatagcaagtttaaataaggctagtccgttatcaacttgaaaaagtggcaccgagtcggtgcttttttt

SEQ ID NO: 30 - DNA, 39 bp, Artificial Sequence
  Sequence from Fig. 11A
tagaacaaaaggtgacaatctgaatgtatttgtgtgaaa

SEQ ID NO: 31 - DNA, 40 bp, Artificial Sequence
  Sequence froom Fig. 11A
ctaaagtagcctgctatgataagttgaaaaagggtttgta

SEQ ID NO: 32 - DNA, 40 bp, Artificial Sequence
  Sequence from Fig. 11A
tagaacaaaaggtgacaaataagttgaaaaagggtttgta

SEQ ID NO: 33 - DNA, 20 bp, Artificial Sequence
  oligonucleotide
taatttgaagctggacccta

SEQ ID NO: 34 - DNA, 20 bp, Artificial Sequence
  oligonucleotide
gtctaggagagtaaagtgat

(19) United States
(12) Patent Application Publication
(10) Pub. No.: US 2023/0024301 A1
(43) Pub. Date: Jan. 26, 2023

(54) TREATMENT OF DISEASES CAUSED BY FRAME SHIFT MUTATIONS

(71) Applicant: Helmholtz Zentrum München Deutsches Forschungszentrum für Gesundheit und Umwelt (GmbH), Neuherberg (DE)

(72) Inventors: Christian Kupatt, Munich (DE); Wolfgang Wurst, Munich (DE); Dong-Jiunn Jeffery Truong, Freising (DE)

(21) Appl. No.: 17/765,804

(22) PCT Filed: Oct. 2, 2020
(86) PCT No.: PCT/EP2020/077634

§ 371 Date: Mar. 31, 2022

(30) Foreign Application Priority Data
Oct. 2, 2019 (LU) LU 101423

(51) Int. Cl. C12N 15/861; C07K 14/47; A61P 21/00

(52) U.S. Cl. CPC C12N 15/861; C07K 14/4708; A61P 21/00

(57) ABSTRACT

The present invention relates a vector system and a vector system for use in a method of treating a disease, each comprising a first vector and a second vector. The present invention further relates to the first vector, the second vector and a combination of the first vector and the second vector. In addition, the present invention relates to a pharmaceutical composition comprising the vector system of the invention or the combination of the invention.

### CROSS-REFERENCE TO RELATED APPLICATIONS

The present application claims the benefit of priority of Luxemburg Patent Application No. 101423 filed 2 Oct. 2019, the content of which is hereby incorporated by reference it its entirety for all purposes.

### TECHNICAL FIELD OF THE INVENTION

The present invention relates a vector system and a vector system for use in a method of treating a disease, wherein the vector system comprises a first vector and a second vector. The present invention further relates to the first vector, the second vector and a combination of the first vector and the second vector. In addition, the present invention relates to a pharmaceutical composition comprising the vector system of the invention or the combination of the invention.

### BACKGROUND

Duchenne muscular dystrophy represents the most frequent hereditary childhood myopathy, leading to progressive muscle degeneration and weakness, and to premature death due to respiratory and cardiac involvement. The vast majority of patients carry frameshift mutations in the DMD gene encoding dystrophin (DMD), are based mainly on exon deletions^{3,4}. The X-chromosomal location of DMD renders 1 in 3,500 to 5,000 male newborns affected⁵.

Antisense oligonucleotide (AON)-mediated exon skipping aimed at reframing DMD transcripts⁶ has already been translated into clinical trials^{7,8}. However, AONs, though initially efficient in a dose-dependent manner⁶, offer only temporary and limited efficacy of DMD expressions. Endonuclease-based gene editing strategies provide a more efficient and permanent genomic correction, as demonstrated in mdx mouse models¹⁰⁻¹⁴.

Recently, intravenous (i.v.) application of AAV9 delivering CRISPR/Cas9 components in a beagle model of DMD (exon 50 deficiency) proved successful in restoring expression of a shortened dystrophin in various muscles, including the heart¹⁵. However, functional data have not been reported as of yet.

Consequently, there still is a need for therapies of diseases caused by frameshift mutations such as deletion of an exon of a gene. The technical problem of the invention is to comply with this need.

### SUMMARY OF THE INVENTION

The technical problem is solved by the subject-matter as defined in the claims. As shown in Example 1, the inventors could surprisingly show that a vector system comprising two vectors, each comprising a fragment of an endonuclease that is fused to a split intein can be used in the treatment of DMD as an exemplary disease caused by a frameshift mutation. The inventors could for the first time show the successful application of excision of an exon to restore the reading frame of a gene.

Accordingly, the present invention relates to a vector system for use in a method of treating a disease, the vector system comprising
(a) a first vector comprising a nucleic acid sequence encoding:
   (i) a first fragment of an endonuclease,
   (ii) a first fragment of an intein, and
   (ii) a first guide RNA (gRNA); and
(b) a second vector comprising a nucleic acid sequence encoding:
   (i) a second fragment of the endonuclease,
   (ii) a second fragment of the intein, and
   (ii) a second guide RNA (gRNA);
      wherein the first gRNA binds to a region, which is located 5′ to a sequence of interest comprised in a nucleic acid sequence in the genome, preferably DNA, of a target cell, wherein the second gRNA binds to a region located 3′ to the sequence of interest comprised in the nucleic acid sequence in the genome, preferably DNA, of a target cell;
      wherein the first fragment and the second fragment of the intein are capable of associating into a functional intein,
      wherein the functional intein is capable of ligating the first and the second fragment of the endonuclease to form a functional endonuclease;
      wherein the functional endonuclease is capable of excising the sequence of interest.

The endonuclease may be Cas9, preferably Streptococcus pyogenes Cas9 (SpCas9), more preferably the Cas9 comprises or has an amino acid sequence as set forth in SEQ ID NO: 1 or an amino acid sequence having at least 60%, 70%, 80%, 85%, 90%, 95%, 98%, 99% or 100% sequence identity to a sequence as shown in SEQ ID 1.

The first fragment of the nuclease may comprise an amino acid sequence selected from the group consisting of SEQ ID NOs: 2 and 3 or an amino acid sequence having at least 60%, 70%, 80%, 85%, 90%, 95%, 98%, 99% or 100% sequence identity to a sequence selected from the group consisting of SEQ ID NOs: 2 and 3.

The second fragment of the nuclease may comprise an amino acid sequence selected from the group consisting of SEQ ID NOs: 4 and 5 or an amino acid sequence having at least 60%, 70%, 80%, 85%, 90%, 95%, 98%, 99% or 100% sequence identity to a sequence selected from the group consisting of SEQ ID NOs: 4 and 5.

The intein may be Npu of SEQ ID 6 or an amino acid sequence having at least 60%, 70%, 80%, 85%, 90%, 95%, 98%, 99% or 100% sequence identity to a sequence as shown in SEQ ID 6, NrdJ-1 of SEQ ID 7 or an amino acid sequence having at least 60%, 70%, 80%, 85%, 90%, 95%, 98%, 99% or 100% sequence identity to a sequence as shown in SEQ ID 7 and gp-41 as shown in SEQ ID 8 or an amino acid sequence having at least 60%, 70%, 80%, 85%, 90%, 95%, 98%, 99% or 100% sequence identity to a sequence as shown in SEQ ID 8.

The first fragment of the intein may comprise or have an amino acid sequence selected from the group consisting of SEQ ID NOs: 9-11 or an amino acid sequence having at least 60%, 70%, 80%, 85%, 90%, 95%, 98%, 99% or 100% sequence identity to a sequence selected from the group consisting of SEQ ID NOs: 9-11.

The second fragment of the intein may comprise or have an amino acid sequence selected from the group consisting of SEQ ID NOs: 12-14 or an amino acid sequence having at least 60%, 70%, 80%, 85%, 90%, 95%, 98%, 99% or 100% sequence identity to a sequence selected from the group consisting of SEQ ID NOs: 12-14.

The first and/or the second vector may be a viral vector.

The viral vector preferably is an adeno-associated virus or lentivirus.

The viral vector preferably is an adeno-associated virus (AAV).

The AAV preferably is selected from the group consisting of AAV1, AAV2, AAV3, AAV4, AAV5, AAV6, AAV7, AAV8, AAV9, AAV10, AAV11 or any combination thereof. More preferably, the AAV is AAV1, AAV5, AAV6 or AAV9, most preferably AAV9.

The viral vector may be coated with a dendrimer. The dendrimer preferably is a PAMAM (Poly(amidoamine)). The dendrimer preferably is a 2^{nd} generation PAMAM.

The nucleic acid of the first and/or the second vector may further comprise:
(iv) a nuclear localization signal, preferably comprising or having the sequence selected from the list consisting of SEQ ID NOs: 15-20 or an amino acid sequence having at least 60%, 70%, 80%, 85%, 90%, 95%, 98%, 99% or 100% sequence identity to a sequence selected from the list consisting of SEQ ID NOs: 15-21.

The first fragment of the nuclease and the second fragment of the intein and the nucleic acid(s) encoding the first and/or the second gRNA may be operatively coupled to a promoter, wherein the promoter(s) preferably is/are inducible.

The promoter that is operatively coupled to the first fragment of the nuclease and/or the second fragment of the intein may be selected from the group consisting of CBH, preferably as depicted in SEQ ID NO: 22, B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, Mb promoter, NphsI promoter, SP-B promoter, SYN1 promoter or WASP promoter.

The promotor that is operatively coupled to the first and/or the second gRNA may be an RNA polymerase III promoter, preferably selected from the list consisting of U6, H1 and 7SK, more preferably the promoter is U6 as depicted in SEQ ID NO: 21.

The method (of treating a disease) may further comprise:
administering to the subject the first vector; and
administering to the subject the second vector.

The method (of treating a disease) may further comprise:
excising the sequence of interest.

The first and the second vector may be administered to the patient simultaneously or sequentially.

The first and the second vector may be administered to the patient sequentially, preferably with a time delay of at least 5 minutes, at least 10 minutes, at least 15 minutes, at least 30 minutes, of at least 1 hour, at least 2 hours, at least 3 hours, at least 4 hours, at least 8 hours, at least 12 hours, at least 24 hours, at least 48 hours, at least 72 hours, at least 1 week or at least 2 weeks.

The subject may be a mammal, preferably a human or a pig, more preferably a human.

The first and the second vectors may be administered systemically, enterally, parenterally, intravenously, intra-arterially, topically, intraperitoneally, intramuscularly, intradermally, intrathecally, intravitreally, subcutaneously, transdermally and/or transmucosally.

The disease may be selected from the group consisting of Duchenne muscular dystrophy, hereditary myopathy with early respiratory failure, early-onset myopathy with fatal cardiomyopathy, core myopathy with heart disease, centronuclear myopathy, limb-girdle muscular dystrophy type 2J, familial dilated cardiomyopathy 9, hypertrophic cardiomyopathy and tibial muscular dystrophy, preferably Duchenne muscular dystrophy.

The disease may be a muscular disease, preferably the disease is Duchenne muscular dystrophy, preferably characterized by a deletion of exon 52 of the dystrophin gene.

The nucleic acid sequence of interest may be exon 51 of the dystrophin gene, preferably exon 51 comprises or has a sequence of 23 or 24 or a nucleic acid sequence comprising or having at least 60%, 70%, 80%, 85%, 90%, 95%, 98%, 99% or 100% sequence identity to a sequence as shown in SEQ ID 23 or 24.

The first gRNA may comprise a nucleic acid sequence as set forth in any of SEQ ID NOs: 25 or 26 and/or the second gRNA may comprise a nucleic acid sequence as set forth in any of SEQ ID NOs: 25 or 26 or a nucleic acid sequence having at least 60%, 70%, 80%, 85%, 90%, 95%, 98%, 99% or 100% sequence identity to a sequence as shown in SEQ ID NOs: 25 or 26 and/or the second gRNA comprises a nucleic acid sequence as set forth in any of SEQ ID NOs: 27 or 28 or a nucleic acid sequence having at least 60%, 70%, 80%, 85%, 90%, 95%, 98%, 99% or 100% sequence identity to a sequence as shown in SEQ ID NOs: 27 or 28.

The deletion of exon 51 of DMD may restore the reading frame of the DMD, thereby enabling the translation of a truncated but functional DMD.

The present invention further relates to a vector system as defined herein.

The present invention further relates to a first vector as defined herein.

The present invention further relates to a second vector as defined herein.

The present invention further relates to a combination of the first vector of the invention and the second vector of the invention.

The present invention further relates to a pharmaceutical composition comprising the vector system of the invention or comprising the combination of the invention.

The present invention further relates to a method for excising a sequence of interest from the genome, preferably DNA, of a subject, comprising the administration of the vector system of the invention, the combination of the invention or the pharmaceutical composition of the invention and thereby excising the sequence of interest from the genome, preferably DNA, of a subject.

### BRIEF DESCRIPTION OF THE DRAWINGS

The invention will be better understood with reference to the detailed description when considered in conjunction with the non-limiting examples and the accompanying drawings, in which:

FIG. 1 shows the genome editing of DMDΔ52 pigs by Cas9-mediated exon 51 excision. (A) Loss of exon 52 in DMDΔ52 pigs leads to an out-of-frame mutation with a premature stop codon, preventing translation of the mutated dystrophin. Cas9-mediated excision of exon 51 corrects the reading frame, resulting in translation of an internally truncated but functional protein. Out of frame exons are illustrated in gray. (B) Dystrophin expression in DMD animals is locally restored at the injection sites after intramuscular (i.m.) AAV9-Cas9-gE51 treatment and dose-dependently detected after intravenous (i.v.) treatment with 2×10¹³ virus particles/kg (low dose) or 2×10¹⁴ virus particles/kg (high dose). Scale bars, 200 μm. (C), RT-PCR reveals editing of the mutated DMD (delEx52) by AAV9-Cas9-gE51, resulting in the del.Ex52+51 mutation. Percentages indicate expression of the corrected dys (del.Ex52+51) relative to total dys (del.Ex52+del.Ex52+51). (D) Immunoblotting shows that high dystrophin expression levels can be achieved locally after i.m. AAV9-Cas9-gE51 injection in limb muscles, while i.v. application provides a widespread expression in both skeletal and breathing muscles. Percentages indicate expression of dystrophin protein relative to WT. Each dystrophin band was normalized to β-actin. (E) Unsupervised hierarchical clustering of normalized LFQ intensity values separates WT, DMD untreated and i.m. treated skeletal muscle samples. The code indicates z-score normalized expression values. (F) Principal component analysis (PCA) clearly separates proteomes from WT, DMD and i.m. treated skeletal muscle. Spots represent individual samples. (G,H) Representative schematics of 24-hour behavioral observation of a wildtype, DMD untreated, and DMD i.v. treated pig (G) and quantification of their total standing time (H). (I) Creatine kinase levels of DMD animals are ameliorated by i.v. treatment. *p<0.05, **p<0.01.

FIG. 2 shows that the genome editing of DMDΔ52 restores the structure and function of diseased skeletal muscle. (A) WGA staining (red) of cell borders and DNA labeling (blue) indicate less homogenous fiber size distribution and abnormal central nuclei location in DMD skeletal muscle, which can be ameliorated by systemic AAV9-Cas9-gE51 treatment. Scale bars, 25 μm. (B) Minimum diameter of cross-sectional fibers is reduced in intramuscularly (i.m.) AAV9-Cas9-gE51-treated animals. (C) Percentage of fibers with centralized nuclei in diverse skeletal muscles of wildtype (WT), DMD untreated and DMD AAV9-Cas9-gE51 treated animals. (D) After intravenous (i.v.) G2-AAV9-Cas9-gE51 application, skeletal muscles contain a higher density of CD31⁺ capillaries than untreated DMD muscle fibers. (E) More CD14⁺ inflammatory cells were found in untreated DMD than in i.v. treated muscle tissue. (F,G) Interstitial fibrosis was reduced upon i.v. treatment in both peripheral muscle and diaphragm. Scale bars in (F), 50 μm. (H,I) Functionally, the twitch amplitude after common peroneal nerve stimulation (cf. Methods) was increased in i.v. treated animals (H), as was tetanic contraction after 1.5 sec stimulation at 50 Hz (i). *p<0.05, **p<0.01, ***p<0.001 (Mann-Whitney U non-parametric test in B).

FIG. 3 shows that the genome editing of DMDΔ52 improves survival and reduces cardiac arrhythmogenic vulnerability. (A) Kaplan-Meier curve indicating significantly increased survival time in intravenously (i.v.) G2-AAV9-Cas9-gE51 treated (n=6) compared to untreated (n=13) DMD animals (p<0.005). (B) Ejection fraction (EF), contraction velocity (dP/dtₘₐₓ) and relaxation velocity (dP/dtₘᵢₙ) indicate systolic, but not diastolic impairment of DMD heart (wildtype, n=3; untreated DMD, n=3; i.v. treated DMD, n=4). *p<0.05 (C) High-resolution electrophysiological mapping demonstrating normal excitation amplitude (>1.3 mV, violet) in wildtype myocardium but widespread areas of low amplitude excitation (<1.3 mV, yellow) or scar (<0.3 mV, red) in untreated DMD animals. I.v. application of G2-AAV9-Cas9-gE51 partially restored the excitation amplitude (FIG. 9b). (D) Interstitial fibrosis levels in corresponding apical regions of wildtype, untreated DMD and i.v. G2-AAV9-Cas9-gE51 treated DMD hearts. Scale bar, 200 μm. (E-G) Spontaneous single-cell calcium transients were recorded in myocardial slices loaded with Fluo-4 AM. Representative Ca²⁺ transients (E) show increased time to peak and transient amplitude durations (delta F/F₀) in untreated DMD slices (F). Subcellular analysis was performed by measuring Fluo-4 fluorescence (A.U.) over time in 4 regions of interest (ROI) within a single cell; scale bars, 10 μm (G). DMD untreated samples revealed unsynchronized cytosolic Ca²⁺ waves. These pathological features were virtually abolished in i.v. injected animals (E-G). All data presented as mean±SD, *p<0.05, **p<0.01, n>8 cells/group (2 wildtype, 2 untreated DMD, 3 i.v. injected DMD animals).

FIG. 4 shows that the somatic genome editing of human DMDΔ52 rescues disease phenotypes of skeletal and cardiac muscle cells from patient-specific iPSCs. (A) Skeletal myotube formation, defective in myoblasts differentiated from hDMDΔ52 hiPSCs, is rescued by transduction with serotype 6 AAVs coding for an intein-split Cas9 and gRNAs designed for DMD exon 51 excision. (B) RT-qPCR analysis of skeletal myotube markers 7-14 days after myotube induction in myoblasts of all groups; n=4-8/group; **p<0.01; ***p<0.001. (C) Immunofluorescence analysis of myosin heavy chain β (MyHC-β), α-actinin and dystrophin 14 days after myotube induction in myoblasts of all groups; scale bars, 100 μm. Inlets show multinucleation (top) and sarcomeric striations (bottom); scale bars, 25 μm. (D) Percentage of MyHC-β⁺ cells 7-14 days after myotube induction in myoblasts of all groups; n=2-3/group; ***p<0.001. (E) Cardiomyocytes differentiated from DMDΔ52 hiPSCs were transduced with serotype 6 AAVs coding for an intein-split Cas9 and gRNAs designed for DMD exon 51 excision as well as either eGFP or mCherry. (F) Representative single-cell Ca²⁺ traces of hiPSC-derived cardiomyocytes of all groups (1 Hz pacing). (G) Ca²⁺ transient durations at 90%, peak magnitude (TD₉₀) in hiPSC-derived cardiomyocytes of all groups (1 Hz pacing); n=3-6/group; **p<0.01; ***p<0.001. (H) Representative single-cell Ca²⁺ trace showing an arrhythmic event in a hDMDΔ52 cardiomyocyte and percentage of cells measured without an arrhythmic event occurring in hiPSC-derived cardiomyocytes of all groups; n=3/group, **p<0.01.

FIG. 5 shows the testing of gRNAs for deletion of pig DMD exon 51. (A) For selecting appropriate gRNAs with high on-target activity, 6 different gRNAs have been cloned into pAAV-N-Cas9 and pAAV-C-Cas9 vector respectively and tested in 6 gRNA combinations by transient transfection in a porcine cell line. (B) The best performing combination (gRNA5′-1 and gRNA3′-1, in the following referred to Ss_DMD_gRNA_Intron50 and Ss_DMD_gRNA_Intron 51; sequences are listed in Table 2) has been selected based on PCR screening. (C) Schematic representation of the split-Cas9 system. Each AAV construct harbours one DMD-specific gRNA under the control of an U6 promoter and one half of the intein-fused Cas9 nuclease under the control of a CBh promoter. (D,E) AAVs were tested in vitro in primary porcine myoblasts for their ability to con-infect. Immunocytochemistry for both the N- as well as the C-terminal peptide of Cas9 is shown in (D). Since the in vitro infection efficacy was expectable low, primary porcine kidney cells have been used to test for gene editing ability. Results of the PCR analysis of genomic editing events are depicted in (E). Scale bar in (D), 15 mm.

FIG. 6. system analyses of DMD exon 51 deletion in pig skeletal muscles and heart after injection of AAV9-Cas9-gE51. PCR analysis of genomic events of DMD editing in tissue samples of DMD pigs treated with CRISPR/Cas9 expressing AAV demonstrates the successful deletion of exon 51. Note the diverse levels of efficacy within the same tissue after intra muscular (i.m.) injection (a) and among different muscles upon high dose systemic (i.v.) injection (b). Correct cutting events by the two gRNAs up- and downstream of exon 51 (Ss_DMD_gRNA_Intron50 and Ss_DMD_gRNA_Intron51) are indicated by a shortening of the respective PCR amplicon from 1988 bp to about 959 bp. Reverse transcriptase PCR (RT-PCR) analysis of DMD mRNA expression and mass spectrometry based quantification of dystrophin protein (Dys) expression are shown for the same samples. RT-PCR was performed under non-restrictive conditions using primer SsDMD_cDNA_Ex50_for and SsDMD_cDNA_Ex53_rev (for details see Table 5). Mass spectrometry based identification of Dys was conducted as detailed in FIG. 7. c, Reverse transcriptase PCR (RT-PCR) analysis of DMD mRNA expression in various cardiac tissue samples of a DMD pig treated systemically (i.v.) with a high dose of CRISPR/Cas9 expressing AAV. Expression of the exon 52 deprived mRNA (del.Ex.52) indicated by a 452 bp amplicon can be detected in most samples. The gene-edited form deprived of exon 51 (del.Ex.52+51, 219 bp amplicon) is additionally expressed with a ratio to total DMD transcript ranging from 0.5 to 3. e, Western blot analysis of dystrophin protein expression in WT heart extract at different dilutions and indicated heart regions of a DMD pig i.v. treated with a high dose of CRISPR/Cas9 expressing AAV. Gapdh was used as a loading control. d, Representative immunofluorescence images for dystrophin expression (Dys) in the heart of WT and DMD animals with or without intravenous (i.v.) injection of high dose G2-AAV9-Cas9-gE51. Wheat germline agglutinin (WGA, red) marks the plasmamembrane. Scale bars, 20 μm.

FIG. 7 shows that the coating of AAV9 with G2 PAMAM dendrimers enhances cardiotropism. (A) dTomato pigs were used for assessment of efficacy of cardiac Cre-expression by a switch from red to green fluorescence. (B) 3 weeks after intravenous application of 2×10¹³ virus particles/kg AAV9-Cre (upper panels), almost no Cre-activity (=green fluorescence) was detectable. However, when coating of the same amount of AAV9-Cre was performed with G2-PAMAM dendrimers (45 ng/10¹³ vps), a broad transduction of cardiomyocytes was achieved. Scale bar, 100 μm.

FIG. 8. Evaluation of fibrosis in DMD muscle after AAv9-Cas9-gE51 treatment. a, quantitative proteome analysis of WT, DMD deficient and corresponding i.m. treated skeletal muscle samples reveal reduction of collagen and fibronectin levels after i.m. treatment. b, c, Quantification of hydroxyproline levels in WT and DMD skeletal muscle (b) and heart (c) samples with and without i.v. treatment. *p<0.05 (one way ANOVA).

FIG. 9 shows the in-vivo electro-mapping and ex-vivo single-cell Ca2+ analyses of DMD hearts. (A) Schematic of the 18.5F IntellaMap-Orion catheter used for high-resolution 3D-mapping. It contains 64 flat microelectrodes (0.8 mm diameter) in a basket configuration with 8 splines. The basket is steerable in 2 directions and can be opened and closed to provide appropriate wall contact for detection of electrophysiological signals. (B,C) One animal of the untreated DMD group, 2 of the high dose treated DMD group and 3 animals of the control group finally underwent an electrophysiological studies and LV endocardial mapping. Mean voltage revealed a decrease in untreated DMD compared to wildtype controls, which was partially rescued by high dose i.v. G2-AAV9-Cas9-gE51 application (B). Conversely, the endocardial scar area, defined as displaying <0.3 mV on the bipolar voltage map and being extensive in the untreated DMD heart, decreased with high-dose G2-AAV9-Cas9-gE51 i.v. application (C). (D) For ex vivo single-cell Ca2+ measurements, left ventricular transmural sections were taken and further processed to 1.0×0.5 cm myocardial tissue slices with 300 μm thickness by vibratome cutting. Myocardial tissue slices were placed in biomimetic culture chambers where tissue was subjected to physiological preload of 1 mN according to fiber direction and continuous electrical field stimulation at 0.5 Hz (50 mA pulse current, 1 ms pulse duration). Eighteen to twenty-four hours post sectioning, myocardial tissue slices were loaded with 3 μM Fluo-4 AM and spontaneous single cell (ROI) or subcellular single-cell (ROI1-4) calcium transients were recorded.

FIG. 10. Colocalisation of dystrophin-associated glycoprotein complex (DGC) and restored dystrophin in DMD pig skeletal muscle after i.m. and i.v. injection of G2-AAV9-Cas9-gE51. a, Confocal immunofluorescence detection of g-sarcoglycan colocalising with dystrophin and spectrin at the sarcolemma. Whereas g-sarcoglycan (red) is substantially diminished in untreated DMD biceps femoris, i.m. injection of G2-AAV9-Cas9-gE51 rescued partial colocalisation with restored dystrophin (green) (upper i.m. series), whereas it failed to recruit gsarcoglycan to areas distant to the injection site (lower i.m. series). Dystrophin restoration upon intravenous injection of G2-AAV9-Cas9-gE51 also induced a more widespread partial colocalisation of the DGC. Spectrin staining confirms sarcolemmal integrity. Scale bars, 200 μm (left 20× merge column), 20 μm (right merge column). b, Confocal immunofluorescence detection of β-dystroglycan (red) with dystrophin was also abundant in the sarcolemma of wildtype biceps femoris, but decreased in dystrophin-deficient muscle in an irregular manner. Restoration of dystrophin by i.m. and i.v. application of G2-AAV9-Cas9-gE51 recovered partial colocalisation with β-dystroglycan (green) and increased overall sarcolemmal β-dystroglycan expression. Scale bars, 200 μm (left 20× merge column), 20 μm (right merge column), 10 μm (detail column).

FIG. 11 shows the generation of patient-specific DMD iPSC isogenic lines. (A) Schematic representation of DMD exon 52 deletion in the patient-specific hDMDΔ52 hiPSC line. Presence of the mutation was verified by PCR on hDMDΔ52 genomic DNA with primers flanking a 69 kb-long sequence in the presence of exon 52 and amplifying a residual 370 bp-long fragment in the case of deletion. (B) Bright field image of alkaline phosphatase staining performed on hDMDΔ52 hiPSC colonies at passage 6; scale bar, 100 μm. (C) Normal male karyotype was confirmed in hDMDΔ52 hiPSCs at passage 23. (D) Loss of Sendai virus was validated in hDMDΔ52 hiPSCs at passage 13 by RT-PCR analysis of the Sendai vector and viral transgenes OCT4, SOX2, KLF4 and c-MYC using GAPDH as an endogenous control. Uninfected and Sendai-infected PBMCs were used as negative and positive controls, respectively. (E) Immunofluorescence analysis of the pluripotency markers Nanog and TRA-1-81 with DAPI counterstaining in hDMDΔ52 hiPSCs at passage 24; scale bar, 50 μm. (F) RT-qPCR analysis of the pluripotency markers OCT4, SOX2, NANOG, REX1 and TDGF-1 using GAPDH as an endogenous control in hDMDΔ52 hiPSCs (passages 13 and 20). Expression fold change relative to parental patient PBMCs is indicated as mean±SEM; n=2. (G) Germ-layer differentiation potential of hDMDΔ52 hiPSCs was assessed by RT-qPCR analysis of markers of endoderm (SOX7, AFP), mesoderm (CD31, DES, ACTA2, SCL, CDH5) and ectoderm (KRT14, NCAM1, TH, GABRR2) using GAPDH as an endogenous control after 21 days of spontaneous EB differentiation. Expression fold change relative to hiPSCs is indicated as mean±SEM; n=2. (H) DMD exon 51 was excised in hDMDΔ52 hiPSCs using a pair of Cas9/gRNA ribonucleoprotein complexes to generate the clonal isogenic hDMDΔ51-52 line. Deletion was verified by PCR on hDMDΔ51-52 genomic DNA with primers generating a 2 kb-long fragment in the presence of exon 51 and a 1.2 kb-long fragment in case of excision, with unedited hDMDΔ52 hiPSCs as a negative control. (I) Normal karyotype after CRISPR/Cas9 editing was confirmed in hDMDΔ51-52 hiPSCs at passage 14.

FIG. 12 shows the generation of control iPSCs from a healthy, young male donor. (A) Bright field image of alkaline phosphatase staining performed on hiPSC colonies at passage 12; scale bar, 100 μm. (B) Normal male karyotype was confirmed in healthy hiPSCs at passage 21. (C) Loss of Sendai virus was validated in hiPSCs at passage 24 by RT-PCR analysis of the Sendai vector and viral transgenes OCT4, SOX2, KLF4 and c-MYC using GAPDH as an endogenous control. Uninfected and Sendai-infected PBMCs were used as negative and positive controls, respectively. (D) Immunofluorescence analysis of the pluripotency markers Nanog and TRA-1-81 with DAPI counterstaining in hiPSCs at passage 21; scale bar, 50 μm. (E) RT-qPCR analysis of the pluripotency markers OCT4, SOX2, NANOG, REX1 and TDGF-1 in hiPSCs (passages 15 and 21). Expression fold change relative to parental patient PBMCs is indicated as mean±SEM; n=2. (F) Germ-layer differentiation potential of hiPSCs was assessed by RT-qPCR analysis of markers of endoderm (SOX7, AFP), mesoderm (CD31, DES, ACTA2, SCL, CDH5) and ectoderm (KRT14, NCAM1, TH, GABRR2) after 21 days of spontaneous EB differentiation. Expression fold change relative to hiPSCs is indicated as mean±SEM; n=2.

FIG. 13 shows that the direct infection of hDMDΔ52 hiPSC-derived skeletal myoblasts and cardiomyocytes with AAV2/6-Cas9-gE51 restores expression of a full-length re-framed dystrophin. (A) Representative bright field images of skeletal myoblasts obtained from healthy, hDMDΔ52 or hDMDΔ51-52 hiPSCs; scale bars, 100 μm. (B) Expression of the skeletal myoblast markers MyoD (MYOD), MyoG (MYOG) and desmin (DES) was assessed by RT-qPCR in skeletal myoblasts differentiated from healthy, hDMDΔ52 or hDMDΔ51-52 hiPSCs. The expression fold change relative to respective hiPSCs is indicated as mean±SEM; n=2-5 per group; *p<0.05; **p<0.01; ***p<0.001; t-test. (C) Representative fluorescence and bright field (BF) images of hDMDΔ52 hiPSC-derived skeletal myoblasts (top panel) or cardiomyocytes (bottom panel) 6 days after transduction with a pair of serotype 2/6 AAVs each coding for part of an intein-split Cas9 and one of two gRNAs for excision of human DMD exon 51 as well as eGFP or mCherry (AAV2/6-Cas9/gE51-eGFP/mCherry); scale bars, 100 μm. (D) Percentage of double positive (eGFP⁺/mCherry⁺), single positive (eGFP⁺/mCherry⁻ or mCherry⁺/eGFP⁻) and negative (eGFP⁻/mCherry⁻) hDMDΔ52 hiPSC-derived skeletal myoblasts (left) or cardiomyocytes (right) 6 days after transduction with AAV2/6-Cas9/gE51-eGFP/mCherry, indicated as mean±SEM; n=2 per group; N=943 cells (skeletal myoblasts) or 398 cells (cardiomyocytes). (E) DMD exon 51 excision after transduction of hiPSC-derived skeletal myoblasts or cardiomyocytes with AAV2/6-Cas9/gE51 was verified by genomic PCR with primers producing a 2 kb fragment in the presence of DMD exon 51 and a 1.2 kb fragment in case of excision, with untreated hDMDΔ52 hiPSC-derived cells as a control. (F) Dystrophin levels were measured with a capillary-based immunoassay (Wes, ProteinSimple) after skeletal myotube differentiation of healthy myoblasts, untreated hDMDΔ52 myoblasts, hDMDΔ52 myoblasts transduced with AAV2/6-Cas9/gE51 or hDMDΔ51-52 myoblasts (left) and in healthy cardiomyocytes, untreated hDMDΔ52 cardiomyocytes, hDMDΔ52 cardiomyocytes transduced with AAV2/6-Cas9/gE51-eGFP/mCherry and hDMDΔ51-52 cardiomyocytes (right). The assay was performed with an antibody detecting the C-terminus of both the main dystrophin isoform (Dp427) and a shorter isoform (Dp71) and an antibody targeting a-actin as a loading control.

FIG. 14. Coating of AAV9 with G2 PAMAM dendrimers does not affect organ integrity. dTomato pigs were used for assessment of toxic effects 1 week after intravenous application of AAV9-eGFP (2×1014 vg/kg, coated with 450 ng G2 PAMAM). Hematoxylin and eosin staining revealed no morphologic and structural changes of the indicated organs. No influx of inflammatory cells was noted. Concordantly, no alterations were detected by lab chemistry of liver and kidney function as well as inflammatory parameters. Scale bars, 100 μm.

FIG. 15. Local virus injection into coronary vessels can be done either into the coronary artery (left panel), or into the coronary vein accompanying the artery (right panel), each during balloon inflation of the target vessel and the accompanying vessel, to minimize blood flow/drainage and to maximize transduction efficacy.

FIG. 16. Comparison of dystrophin expression in wildtype pig hearts (lower panel, green color), DMD pig hearts lacking dystrophin after 4 weeks of systemic intravenous transduction (middle panel), and DMD pig hearts at 4 weeks after local intravenous transduction (top panel) with G2-AAV9-Cas9-gE51 vector as described in Example 2 (200× magnification in each panel). Blue dots are DAPI-stained cell nuclei.

### DETAILED DESCRIPTION OF THE INVENTION

The present invention is described in detail in the following and will also be further illustrated by the appended examples and figures.

Large genes such as dystrophin or titin comprise a high number of exons. Not all of these exons comprise a number of nucleotides that can be divided by 3, i.e. a deletion of one of these exons might lead to a frameshift mutation. This would then result in a truncated and/or non-functional protein. Alternatively, an exon may also be duplicated, triplicated etc. and could thus induce, e.g. a frameshift mutation or lead otherwise to a non-functional expression product. In addition, a stop codon can occur due to point mutation or small mutations in an exon, also leading to complete dystrophin deficiency. Both, dystrophin and titin are large proteins of the muscle and are involved in the movement of muscles. Thus, nonsense mutations of these proteins lead to severe diseases, which are potentially deadly when organs such as the heart or lung are affected. A prominent example for such a disease is Duchenne muscular dystrophy (DMD).

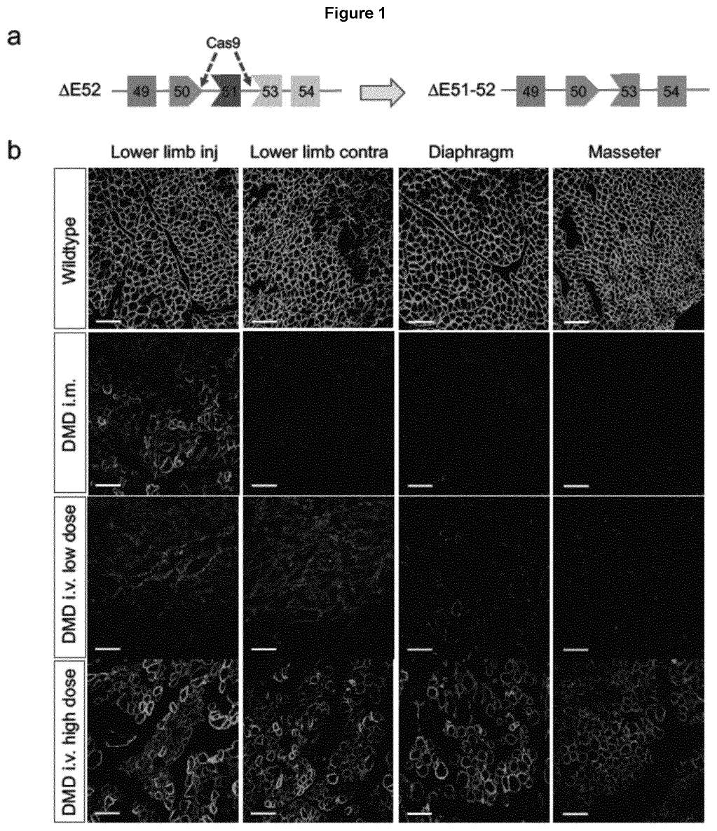

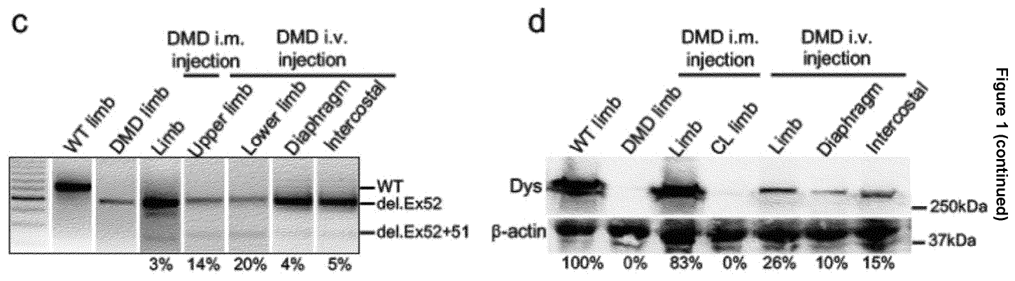

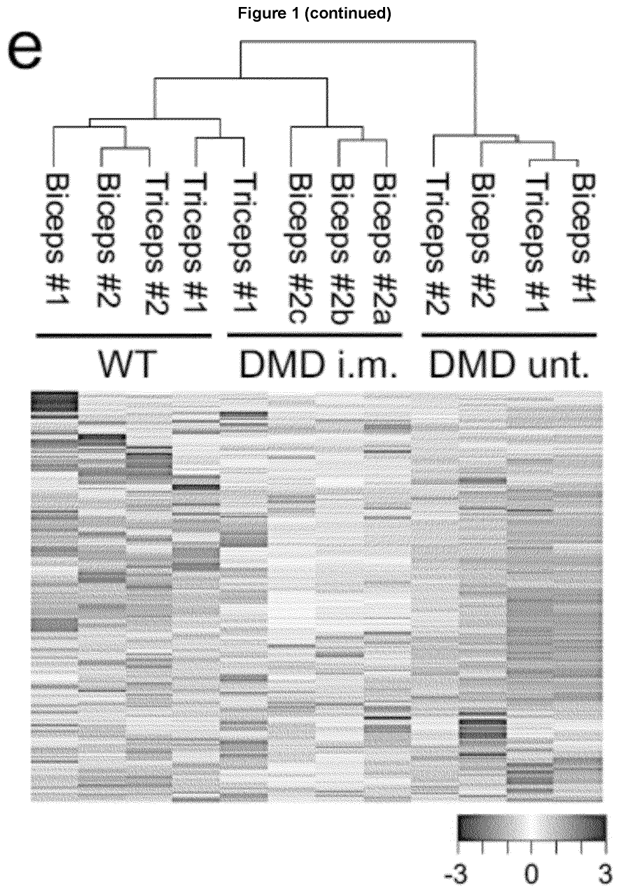

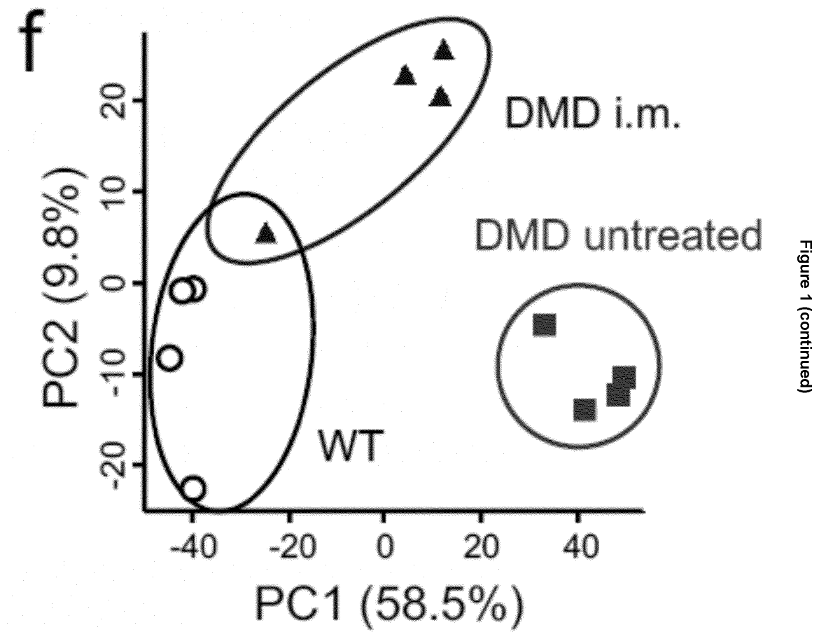

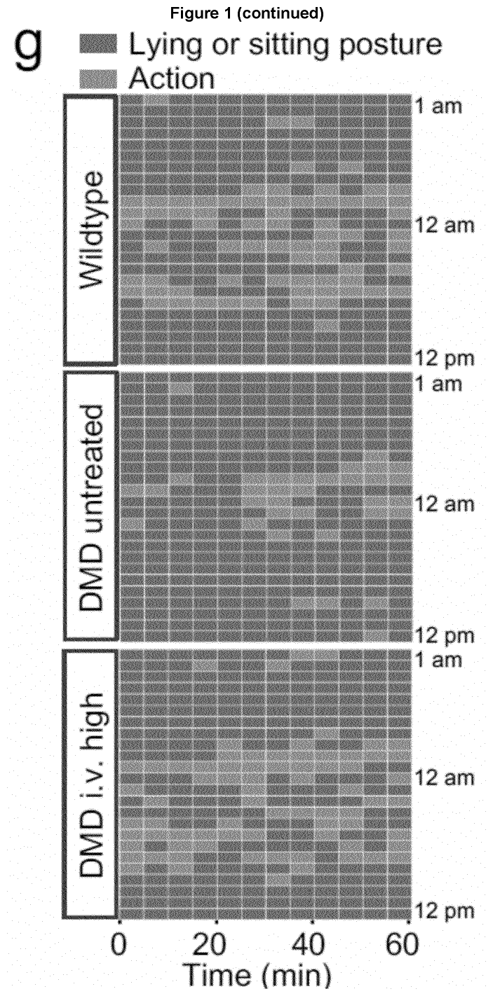

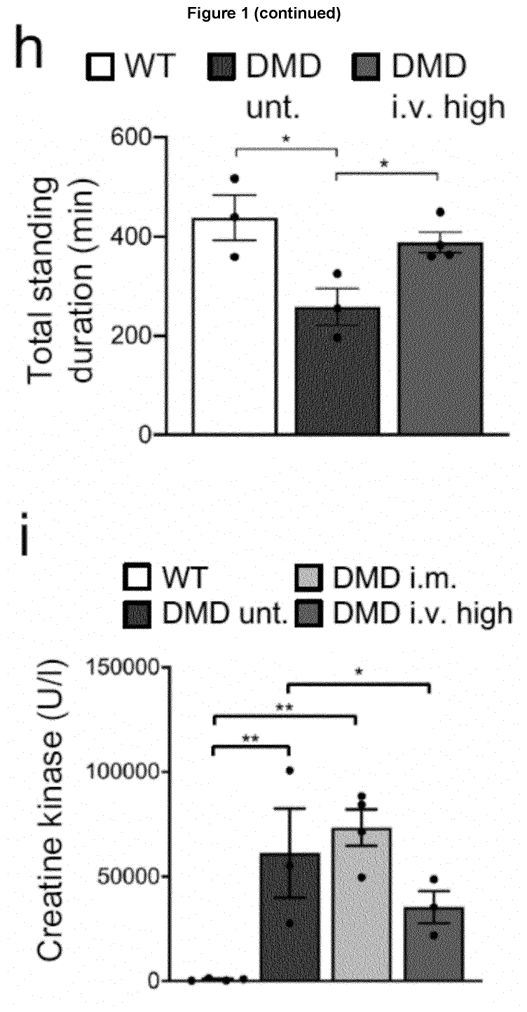

DMD is very often characterized by deletions of one exon such as exon 52. Exon 52 consists of a number of nucleotide that cannot divided by 3, i.e. a deletion of exon 52 leads to a frameshift mutation. This is shown in FIG. 1A. Thus, the inventors aimed at deleting exon 51 to restore the frame of the protein. Because of the size and structure of dystrophin the loss of exons 51 and 52 does not significantly alter the function of the protein. Thus, the truncated dystrophin can replace the “normal” dystrophin comprising all exons of the wild type dystrophin.

The present inventors found an inventive solution for deleting exons of genes that result in a frame shift mutation. In contrast to prior art technologies, such as that disclosed in Amoasii et al.¹⁵, the inventors applied an improved technology. While Amoasii et al. use one AAV for the Cas9 nuclease and another AAV for overexpressing the sgRNA, the present inventors employed a system making use of a split-intein Cas9 vector system that shows efficacy and safety (see Example 1).

In an exemplary embodiment (see example 1), the inventors make use of a split intein Cas9 system: A first AAV comprises the N-terminal fragment of Cas9 fused to a split intein and a second AAV comprises the C-terminal fragment of Cas9 fused to the second split intein fragment. If only one of these AAVs is administered, no functional endonuclease can be generated, which shows the intrinsic safety of this approach. If both, the first and the second AAV are administered, both Cas9-split intein fragments will associate after expression in the host cell. The split intein will then excise itself and thereby fuse the N- and the C-terminal fragment of Cas9 to a functional protein. The Cas9 endonuclease is guided by a sgRNA, which can be comprised in either or both of the first and the second AAV. In Example 1, one sgRNA targets the Cas9 to the 5′ end of exon 51 of DMD and another on the other AAV to the 3′ prime end of exon 51 of DMD. This finally leads to the excision of exon 51 and restores the frame of dystrophin. The inventors successfully applied this approach in DMD-pigs that were genetically altered to miss exon 52. As shown by the inventors, the pigs that were treated with the vector system of the present invention had an improved survival and reduced arrhythmogenic vulnerability. The Inventors could further show the efficacy of this approach in human cells.

This vector system can be easily transferred to other diseases of the same type that e.g. are related to a deletion or mutation (including, but not limited to, duplication, triplication etc.) of an exon that leads to a frameshift and other endonucleases.

Accordingly, the present invention relates to a vector system for use in a method of treating a disease, the vector system comprising (a) a first vector comprising a nucleic acid sequence encoding: (i) a first fragment of an endonuclease, (ii) a first fragment of an intein, and (ii) a first guide RNA (gRNA); and (b) a second vector comprising a nucleic acid sequence encoding: (i) a second fragment of the endonuclease, (ii) a second fragment of the intein, and (ii) a second guide RNA (gRNA); wherein the first gRNA binds to a region, which is located 5′ to a sequence of interest comprised in a nucleic acid sequence in the genome of a target cell, preferably in the DNA, wherein the second gRNA binds to a region located 3′ to a sequence of interest comprised in the nucleic acid sequence in the genome of a target cell, preferably in the DNA; wherein the first fragment and the second fragment of the intein are capable of associating into a functional intein, wherein the functional intein is capable of ligating the first and the second fragment of the endonuclease to form a functional endonuclease; wherein the functional endonuclease is capable of excising the sequence of interest.

A “genome” as used herein may be described as the genetic material of an organism. As such, it may consist of DNA and/or may relate to the total DNA content of a host cell, organism or subject. The genome preferably includes genes (coding regions) and noncoding DNA as well as mitochondrial DNA. The DNA of the genome usually comprises two strands. Genes can be present on each of the two strands, are however always transcribed in 5′ to 3′ direction (in relation to the coding strand). Thus, when relating to the 5′ or 3′ end of the sequence of interest, the present disclosure relates to the 5′ or 3′ end of the coding strand of the sequence of interest. In this context, it is noted that is irrelevant which of the two strands (coding or template strand) the guideRNA is directed, since the endonucleases induce a double strand break. Thus, the first guideRNA may bind to a region located 5′ to the sequence of interest on the coding strand or to a region located 3′ to the sequence of interest on the template strand. Additionally, the second guideRNA may bind to a region located 3′ to the sequence of interest on the coding strand or to a region located 5′ to the sequence of interest on the template strand.

It is obvious to a person skilled in the art that the case in which the first gRNA binds to a region located 3′ to the sequence of interest and the second gRNA binds to a region located 5′ to the sequence of interest is equivalent to the case in which the first gRNA binds to a region located 5′ to the sequence of interest and the second gRNA binds to a region located 3′ to the sequence of interest.

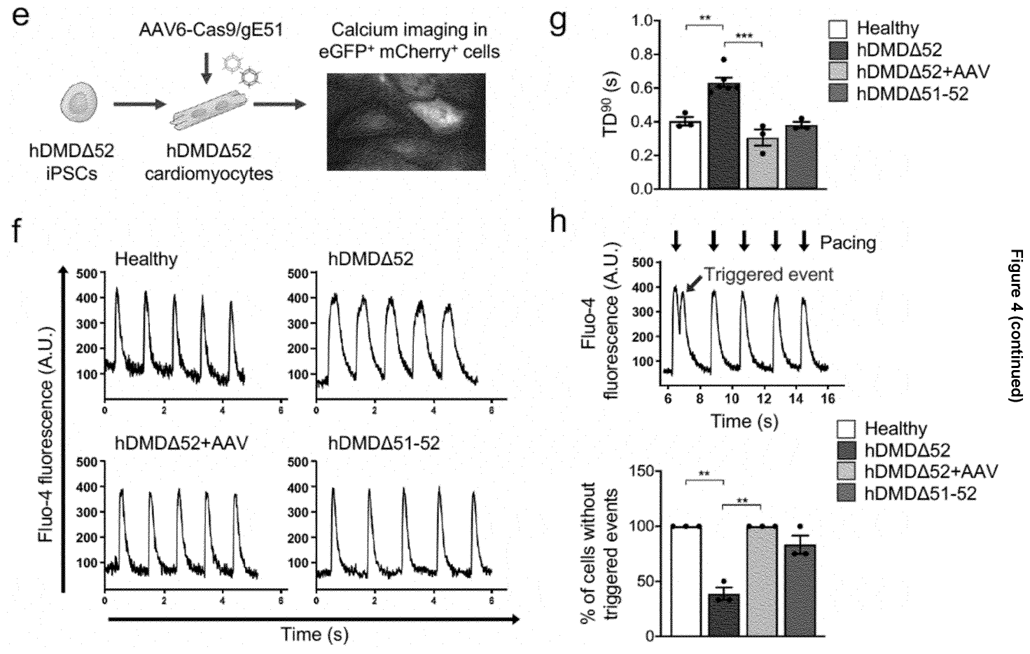

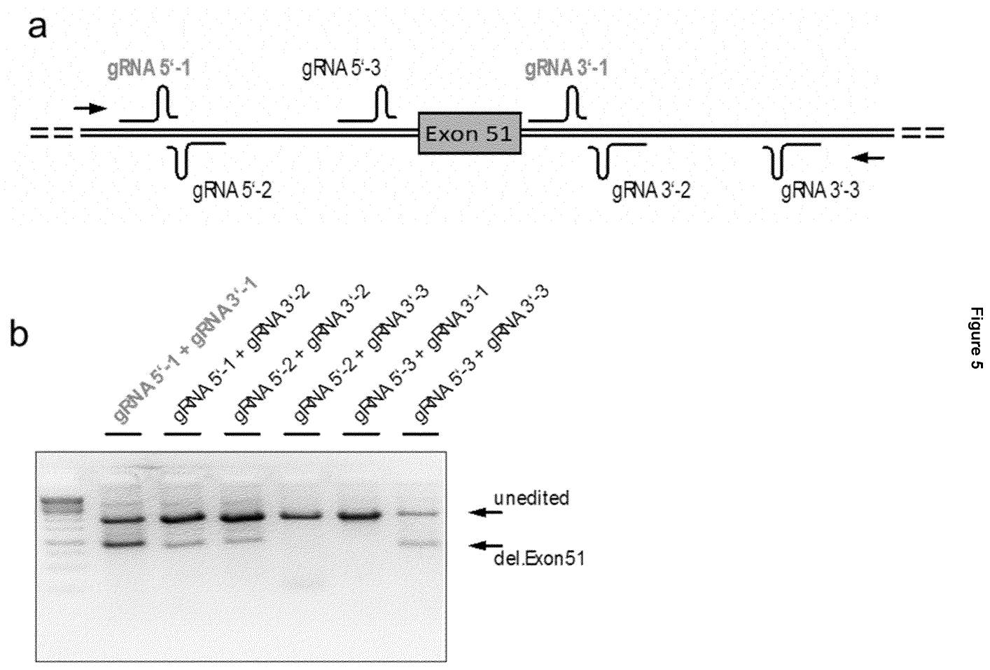

As described herein, the vector system of the present invention comprises two separate vectors, a first vector and a second vector. In principle it is irrelevant on which of the vectors which fragment is comprised. Importantly, there should not be the first and the second fragment of the endonuclease or the first fragment and the second fragment of the intein on the same vector. Which of the both vectors is termed first vector and second vector is not relevant for the scope of the invention. The vector system of the present invention may be used to excise an exon of a gene comprising at least two exons. In a preferred embodiment, the first vector comprises the first fragment of the endonuclease an N-terminal fragment of the endonuclease fused to the first fragment of the intein, which is an N-terminal fragment of the intein, and the second vector comprises as second fragment of the intein an C-terminal fragment of the intein fused to the C-terminal fragment of the endonuclease. Such a domain arrangement is exemplarily shown in FIG. 5D. This domain arrangement allows the fusion of the C-terminus of the N-terminal fragment of the endonuclease to the N-terminus of the C-terminal fragment of the endonuclease.

“Inteins” as used in the context of the present invention and as used throughout the whole description, can be described as protein introns, which are able to auto catalytically splice themselves posttranslationally out of a protein resulting in covalently linked exteins as a scar less gene product. This process may be termed protein splicing. Exteins on the other hand are the remaining portions of the protein after the intein has excised itself out. Within the context of the present invention, the exteins can be described as the first and the second fragment of the endonuclease.

The term “split intein” means in the context of this present invention and as used throughout the whole description, a subset of inteins that are expressed in two separate halves, named in the context of the present invention “first fragment of the intein” and “second fragment of the intein” or alternatively “N-intein” and “C-intein” and catalyze splicing in trans upon association of the two domains. The term “two separate halves” does not mean in this context that the two separated domains of the split intein are even or equally split. Instead, the term also includes any split ratio between the two domains of the split intein, which a person skilled in the art can conceive of. The “split intein” may occur naturally and may also been artificially generated by splitting of contiguous ones. With their unique properties, split-inteins offer improved controllability, flexibility and capability to existing tools based on contiguous inteins.

A “functional intein” as used herein is capable of ligating the first and the second fragment of a protein—a process referred to as intein-mediated protein splicing. Intein-mediated protein splicing typically occurs after the intein-containing mRNA has been translated into a protein. The process begins with an N—O or N—S shift, when the side chain of the first residue (preferably a serine, threonine, or cysteine) of the (N-terminal split) intein portion of the expression product of the specific splice product nucleophilically attacks the peptide bond of the residue immediately upstream (that is, the final residue of the N-extein) to form a linear ester (or thioester) intermediate. A transesterification occurs when the side chain of the first residue of the C-extein, i.e. the amino acid C-terminal to the C-terminal split intein, attacks the newly formed (thio)ester to free the N-terminal end of the intein. This forms a branched intermediate, in which the N-extein and C-extein are attached, albeit not through a peptide bond. The last residue of the intein preferably is an asparagine, and the amide nitrogen atom of this side chain might cleave apart the peptide bond between the intein and the C-extein, resulting in a free intein segment with a terminal cyclic imide. Finally, the free amino group of the C-extein may now attack the (thio)ester linking the N- and C-exteins together. An O—N or S—N shift therefore preferably produces a peptide bond and the functional, ligated protein.

As soon as N- and C-exteins (flanking the intein) are in spatial proximity to each other, the excision process can be initialized by forming a succinimide intermediate. For this process, the presence of several amino acids in fixed positions may be required: Either a cysteine or a serine residue at the N-terminal side of the intein, an asparagine at the C-terminal side of the intein and another cysteine at the beginning of the C-terminal extein may exist. After splicing has taken place, the resulting protein contains the N-extein linked to the C-extein; this splicing product may be also termed an extein.

The ligation activity of the intein can be determined by a person skilled in the art, e.g. by using a Western blot against one terminus of the Cas9 protein, e.g. the N-terminus or the C-terminus. In case, an antibody-binding Cas9 protein larger than the expected terminus size, intein-mediated ligation of the protein halves is viewed as a proof of reassembly. A “functional intein” thus may be seen as an intein that is considered to have ligation activity as determined by said assay.

Examples for split inteins include the Npu intein, the NrdJ-1 intein or the gp41-1 intein, which may all be split and excise the polypeptide that has been fused between the N- and the C-terminus of the split intein.

In one embodiment of the method of the present invention, the first fragment of the split intein comprises or consists of the Npu N-terminal region (SEQ ID NO: 9), the NrdJ-1 N-terminal region (SEQ ID NO: 10), or the gp41-1 N-terminal region (SEQ ID NO: 11) or an amino acid sequence having at least 60%, 70%, 80%, 85%, 90%, 95%, 98%, 99% or 100% sequence identity to a sequence as shown in SEQ ID NOs 9-11, and/or the second fragment of the split intein comprises or consists of the Npu C-terminal region (SEQ ID NO: 12), the NrdJ-1 C-terminal region (SEQ ID NO: 13) or the gp41-1 C-terminal region (SEQ ID NO: 14) or an amino acid sequence having at least 60%, 70%, 80%, 85%, 90%, 95%, 98%, 99% or 100% sequence identity to a sequence as shown in SEQ ID NOs 12-14. In one embodiment of the method of the present invention, the first fragment of the split intein comprises or consists of the Npu N-terminal region (SEQ ID NO: 9) or comprises or consists of an amino acid sequence having at least 60%, 70%, 80%, 85%, 90%, 95%, 98%, 99% or 100% sequence identity to a sequence as shown in SEQ ID 9. In one embodiment of the method of the present invention, the first fragment of the split intein comprises or consists of the NrdJ-1 N-terminal region (SEQ ID NO: 10) or comprises or consists of an amino acid sequence having at least 60%, 70%, 80%, 85%, 90%, 95%, 98%, 99% or 100% sequence identity to a sequence as shown in SEQ ID 10. In one specific embodiment of the method of the present invention, the first fragment of the split intein comprises or consists of the gp41-1 N-terminal region (SEQ ID NO: 11) or comprises or consists of an amino acid sequence having at least 60%, 70%, 80%, 85%, 90%, 95%, 98%, 99% or 100% sequence identity to a sequence as shown in SEQ ID 11. In another embodiment of the method of the present invention, the second fragment of the split intein comprises or consists of the Npu C-terminal region (SEQ ID NO: 12) or comprises or consists of an amino acid sequence having at least 60%, 70%, 80%, 85%, 90%, 95%, 98%, 99% or 100% sequence identity to a sequence as shown in SEQ ID 12. In another embodiment of the method of the present invention, the second fragment of the split intein comprises or consists of the NrdJ-1 C-terminal region (SEQ ID NO: 13) or comprises or consists of an amino acid sequence having at least 60%, 70%, 80%, 85%, 90%, 95%, 98%, 99% or 100% sequence identity to a sequence as shown in SEQ ID 13. In one further embodiment of the method of the present invention, the second fragment of the split intein comprises or consists of the gp41-1 C-terminal region (SEQ ID NO: 14) or comprises or consists of an amino acid sequence having at least 60%, 70%, 80%, 85%, 90%, 95%, 98%, 99% or 100% sequence identity to a sequence as shown in SEQ ID 14. In one further embodiment of the method of the present invention, the first fragment of the split intein consists of the Npu N-terminal region (SEQ ID NO: 9) or comprises or consists of an amino acid sequence having at least 60%, 70%, 80%, 85%, 90%, 95%, 98%, 99% or 100% sequence identity to a sequence as shown in SEQ ID 9. In one further embodiment of the method of the present invention, the first fragment of the split intein consists of the NrdJ-1 N-terminal region (SEQ ID NO: 10) or comprises or consists of an amino acid sequence having at least 60%, 70%, 80%, 85%, 90%, 95%, 98%, 99% or 100% sequence identity to a sequence as shown in SEQ ID 10. In another embodiment of the method of the present invention, the first fragment of the split intein consists of the gp41-1 N-terminal region (SEQ ID NO: 11) or comprises or consists of an amino acid sequence having at least 60%, 70%, 80%, 85%, 90%, 95%, 98%, 99% or 100% sequence identity to a sequence as shown in SEQ ID 11. In one further embodiment of the method of the present invention, the second fragment of the split intein consists of the Npu C-terminal region (SEQ ID NO: 12) or comprises or consists of an amino acid sequence having at least 60%, 70%, 80%, 85%, 90%, 95%, 98%, 99% or 100% sequence identity to a sequence as shown in SEQ ID 12. In one further embodiment of the method of the present invention, the second fragment of the split intein consists of the NrdJ-1 C-terminal region (SEQ ID NO: 13) or comprises or consists of an amino acid sequence having at least 60%, 70%, 80%, 85%, 90%, 95%, 98%, 99% or 100% sequence identity to a sequence as shown in SEQ ID 13. In one embodiment of the method of the present invention, the second fragment of the split intein consists of the gp41-1 C-terminal region (SEQ ID NO: 14) or comprises or consists of an amino acid sequence having at least 60%, 70%, 80%, 85%, 90%, 95%, 98%, 99% or 100% sequence identity to a sequence as shown in SEQ ID 14.

A variety of sequence based alignment methodologies, which are well known to those skilled in the art, can be used to determine identity among sequences. These include, but are not limited to, the local identity/homology algorithm of Smith, F. and Waterman, M. S. (1981) Adv. Appl. Math. 2: 482-89, homology alignment algorithm of Peason, W. R. and Lipman, D. J. (1988) Proc. Natl. Acad. Sci. USA 85: 2444-48, Basic Local Alignment Search Tool (BLAST) described by Altschul, S. F. et al. (1990) J. Mol. Biol. 215: 403-10, or the Best Fit program described by Devereau, J. et al. (1984) Nucleic Acids. Res. 12: 387-95, and the FastA and TFASTA alignment programs, preferably using default settings or by inspection. Alternatively, an alignment may be done manually/visually for amino acids sequences as follows: the percent identity between an amino acid sequence in question (query sequence) and an amino acid sequence of the invention/disclosed in the sequence listing (reference sequence), respectively, as defined herein is determined by pairwise alignment in such a way that the maximum identity is obtained between both amino acid sequences. The identical amino acid residues between both amino acid sequences are counted and divided by the total number of residues of the reference sequence (including positions that do not contain amino acid residues, e.g. one or more gaps) yielding the percentage of identity.

An “endonuclease” as used herein relates to an RNA-guided enzyme that cleaves the phosphodiester bond within a DNA polynucleotide chain. A “functional endonuclease” preferably has said activity of cleaving the phosphodiester bond within a DNA polynucleotide chain. Within the context of the present invention, the endonuclease preferably excises the sequence of interest from the genome, preferably DNA, of a host cell, such as an exon to restore the reading frame of a gene. The endonuclease used in the present invention preferably is a Cas9 endonuclease. The endonuclease used by the present invention may be split into two fragments, a first fragment of the endonuclease and a second fragment of the endonuclease. “Fragment” as used in this context relates to a portion of the endonuclease that is split into two parts or portions (fragments). The term “fragment” does not mean in this context that the two separated parts or portions (fragments) of the endonuclease are even or equally split. Instead, the term also includes any split ratio between the two parts or portions (fragments) of the endonuclease, which a person skilled in the art can conceive of. Both fragments of the endonuclease are ligated by the intein to the functional or “complete” endonuclease. A person skilled in the art knows how to determine whether an endonuclease has the activity of cleaving the phosphodiester bond within a DNA polynucleotide chain or not. Within the context of the invention, a “functional endonuclease” preferably mediates the excision of a sequence of interest, preferably in the genome (DNA) of a host cell. In an exemplary assay, a sample comprising the genome, i.e. nucleic acids such as DNA, obtained from the subject can be analyzed for the presence or absence of the sequence of interest. If the sequence of interest is no longer present in the sample, the endonuclease can be seen as functional. Methods suitable for this assay include, but are not limited to, PCR, qPCR or sequencing.

Various different endonucleases are known to a person skilled in the art. In a preferred embodiment, the endonuclease is Cas9 from Streptococcus pyogenes exemplified in SEQ ID NO: 1. The endonuclease may further be a Cas9 protein having an amino acid sequence having at least 60%, 70%, 80%, 85%, 90%, 95%, 98%, 99% or 100% sequence identity to a sequence as shown in SEQ ID 1. The endonuclease may be split at a specified position leading to two fragments, the first fragment of the endonuclease and the second fragment of the endonuclease. When used within the embodiments of the invention, the endonuclease is divided in two fragments. In one embodiment of the invention, the Cas9 from Streptococcus pyogenes may be split between amino acid positions 573 and 574. In another embodiment of the invention, the Cas9 from Streptococcus pyogenes may be split between amino acid positions 637 and 638.

Accordingly, the first fragment of the endonuclease may comprise an amino acid sequence selected from the group consisting of SEQ ID NOs: 2 and 3 or an amino acid sequence having at least 60%, 70%, 80%, 85%, 90%, 95%, 98%, 99% or 100% sequence identity to a sequence selected from the group consisting of SEQ ID NOs: 2 and 3. In a specific embodiment, the first fragment of the nuclease consists of an amino acid sequence selected from the group consisting of SEQ ID NOs: 2 and 3 or an amino acid sequence having at least 60%, 70%, 80%, 85%, 90%, 95%, 98%, 99% or 100% sequence identity to a sequence selected from the group consisting of SEQ ID NOs: 2 and 3.

Accordingly, the second fragment of the endonuclease may comprise an amino acid sequence selected from the group consisting of SEQ ID NOs: 4 and 5 or an amino acid sequence having at least 60%, 70%, 80%, 85%, 90%, 95%, 98%, 99% or 100% sequence identity to a sequence as selected from the group consisting of SEQ ID NOs: 4 and 5. In a specific embodiment, the second fragment of the nuclease consists of an amino acid sequence selected from the group consisting of SEQ ID NOs: 4 and 5 or an amino acid sequence having at least 60%, 70%, 80%, 85%, 90%, 95%, 98%, 99% or 100% sequence identity to a sequence selected from the group consisting of SEQ ID NOs: 4 and 5.

“Vector” as used herein relates to nucleic acid suitable for transfer and expression of proteins or RNAs encoded by the nucleic acid in a target cell. Vector as used herein may also relate to a virus comprising a nucleic acid suitable for transfer and expression of proteins or RNAs encoded by the nucleic acid in a target cell.

Accordingly, one mode of administration for the vector system of the invention may be in the form of viral particles. Accordingly, the first and/or the second vector of the invention may be a viral vector. The viral vector may be a virus particle comprising a vector encoding the first or the second vector of the invention. Examples for a viral vector include, but are not limited to, adeno-associated virus (AAV) or lentivirus. Preferably, the lentivirus does not integrate into the genome of the target cell. Accordingly, the viral vector may be an AAV. Alternatively, the viral vector may be a lentivirus.

AAV is a small virus that infects humans and some other primate species. The virus causes a very mild immune response. Gene therapy vectors using AAV can infect both dividing and quiescent cells and persist in an extrachromosomal state without integrating into the genome of the host cell. AAV belongs to the genus Dependoparvovirus, which in turn belongs to the family Parvoviridae. The virus is a small (20 nm) replication-defective, nonenveloped virus. Several serotypes of AAV are known to a person skilled in the art. The different serotypes of AAV show different tropism. Accordingly, the AAV may be selected according to the cell type or tissue that is to be genetically modified. Table 1 shows an overview of the tropisms of different AAV serotypes.

TABLE 1
Tropism of AAV Serotypes.

| Tissue | Optimal Serotype |
|---|---|
| CNS | AAV1, AAV2, AAV4, AAV5, AAV8, AAV9 |
| Heart | AAV1, AAV8, AAV9 |
| Kidney | AAV2 |
| Liver | AAV7, AAV8, AAV9 |
| Lung | AAV4, AAV5, AAV6, AAV9 |
| Pancreas | AAV8 |
| Photoreceptor Cells | AAV2, AAV5, AAV8 |
| RPE (Retinal Pigment Epithelium) | AAV1, AAV2, AAV4, AAV5, AAV8 |
| Skeletal Muscle | AAV1, AAV2, AAV5, AAV6, AAV8, AAV9 |

In a preferred embodiment, the AAV is AAV1, AAV2, AAV5, AAV6, AAV8, AAV9 or any combination thereof. In a more preferred embodiment, the AAV is AAV1. In a more preferred embodiment, the AAV is AAV2. In a more preferred embodiment, the AAV is AAV5. In a more preferred embodiment, the AAV is AAV6. In a more preferred embodiment, the AAV is AAV8. In a more preferred embodiment, the AAV is AAV9. The first vector and the second vector do not necessarily be the same AAV but may be different. Preferably however, the first and the second vector are the same AAV such as AAV9.

Thus, the tropism of the (viral) vector determines the target cells that are genetically modified by the vector system of the invention.

As shown by the Inventors (see FIG. 7), PAMAM coating may increase cardiotropism. Accordingly, the first and the second vector may be coated, especially if the vector is a viral vector. Such a viral vector, which may also be described as viral particle comprising the first vector or the second vector of the invention, may be coated with a dendrimer. Dendrimers are repetitively branched molecules. Synonymous terms for dendrimer may include arborols and cascade molecules. A dendrimer is typically symmetric around the core, and often adopts a spherical three-dimensional morphology. Dendrimers are known to a person skilled in the art. Poly(amidoamine), or PAMAM, is perhaps the most well-known dendrimer. The core of PAMAM is a diamine (commonly ethylenediamine), which is reacted with methyl acrylate, and then another ethylenediamine to make the generation-0 (G-0) PAMAM. Successive reactions create higher generations. Lower generations can be thought of as flexible molecules with no appreciable inner regions, while medium-sized (G-3 or G-4) do have internal space that is essentially separated from the outer shell of the dendrimer. Very large (G-7 and greater) dendrimers can be thought of more like solid particles with very dense surfaces due to the structure of their outer shell. The functional group on the surface of PAMAM dendrimers is ideal for click chemistry, which gives rise to many potential applications. Accordingly, the viral vector is preferably coated with a dendrimer. More preferably, the dendrimer is a PAMAM (Poly(amidoamine)). Most preferably, the dendrimer is a 2^{nd} generation PAMAM. A person skilled in the art is aware how to coat a vector with a dendrimer coating. An exemplary method is disclosed in Vetter et al. (18).

A “disease” within the meaning of the present invention relates to any disease that may be treated by the deletion of a sequence of interest such as an exon. The disease may relate to a disease that is caused by the deletion of an exon of a gene, which induces a frameshift mutation. This frameshift mutation may lead to a non-functional protein or to a protein that is not expressed, e.g. because of a stop codon induced by the frameshift mutation. Non-limiting examples for genes that may be affected by such a mutation are titin and dystrophin. Both of which are important for the function of muscles. Diseases caused by frameshift mutations of titin or dystrophin may include Duchenne muscular dystrophy, hereditary myopathy with early respiratory failure, early-onset myopathy with fatal cardiomyopathy, core myopathy with heart disease, centronuclear myopathy, limb-girdle muscular dystrophy type 2J, familial dilated cardiomyopathy 9, hypertrophic cardiomyopathy and tibial muscular dystrophy. In addition or alternatively, also proteins characterized by repetitive protein domains such as immunoglobulins may be affected. The disease within the meaning of the invention may further relate to diseases that are caused by the presence of an exon, which is not present in a healthy subject, e.g. a duplication, triplication etc. of an exon. Such a duplication, triplication etc. may be present in the dystrophin gene and/or may also lead to Duchenne muscular dystrophy.

In a preferred embodiment, the disease is Duchenne muscular dystrophy (DMD). DMD is a severe type of muscular dystrophy characterized by muscle weakness usually beginning around the age of four in boys and worsens quickly. Typically muscle loss occurs first in the thighs and pelvis followed by those of the arms. Most patients are unable to walk by the age of 12. The disorder is X-linked recessive. About two thirds of cases are inherited from a person's mother, while one third of cases are due to a new mutation. It is caused by a mutation in the dystrophin gene at locus Xp21, located on the short arm of the X chromosome and is inherited in an X-linked recessive pattern. Dystrophin is responsible for connecting the cytoskeleton of each muscle fiber to the underlying basal lamina (extracellular matrix), through a protein complex containing many subunits. The absence of dystrophin permits excess calcium to penetrate the sarcolemma (the cell membrane). Alterations in calcium and signaling pathways cause water to enter into the mitochondria, which then burst.

The present invention makes use genome-targeting nucleic acids that can direct the activities of an associated endonuclease to a specific target sequence within a target nucleic acid. The genome-targeting nucleic acid can be RNA. A genome-targeting RNA is referred to as a “guide RNA” or “gRNA” herein. A guide RNA can comprise at least a spacer sequence that hybridizes to a target nucleic acid sequence of interest, and a CRISPR repeat sequence. In Type II systems, the gRNA also comprises a second RNA called the tracrRNA sequence. In the Type II guide RNA (gRNA), the CRISPR repeat sequence and tracrRNA sequence hybridize to each other to form a duplex. In the Type V guide RNA (gRNA), the crRNA forms a duplex. In both systems, the duplex can bind a site-directed polypeptide, such that the guide RNA and site-direct polypeptide form a complex. The genome-targeting nucleic acid can provide target specificity to the complex by virtue of its association with the site-directed polypeptide. The genome-targeting nucleic acid thus can direct the activity of the site-directed polypeptide. The functionality of a genome-targeting nucleic acid can be tested by analyzing the DNA that should have been modified. If the desired modification is present, the genome-targeting nucleic acid(s) target the endonuclease to the correct position and excises the correct sequence of interest from the genome. Suitable methods include, but are not limited to, Mismatch cleavage assay, Sequence trace decomposition analysis, Indel Detection by Amplicon Analysis (IDAA), Digital PCR, Immunofluorescence analysis or Clonal analysis.

Each guide RNA can be designed to include a spacer sequence complementary to its genomic target sequence. Exemplary spacers are shown in SEQ ID NO: 25-28 (see also Table 2). For example, each of the spacer sequences in the Sequence Listing can be put into a single strand guide RNA (sgRNA) (e.g., an RNA chimera) or a crRNA (along with a corresponding tracrRNA). See Jinek et al., Science, 337, 816-821 (2012) and Deltcheva et al., Nature, 471, 602-607 (2011). The genome-targeting nucleic acid can be a double-molecule guide RNA. The genome-targeting nucleic acid can be a single-molecule guide RNA.

A double-molecule guide RNA can comprise two strands of RNA. The first strand comprises in the 5′ to 3′ direction, an optional spacer extension sequence, a spacer sequence and a minimum CRISPR repeat sequence. The second strand can comprise a minimum tracrRNA sequence (complementary to the minimum CRISPR repeat sequence), a 3′ tracrRNA sequence and an optional tracrRNA extension sequence.

A single-molecule guide RNA (sgRNA) in a Type II system can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. The optional tracrRNA extension can comprise one or more hairpins.

A single-molecule guide RNA (sgRNA) in a Type V system can comprise, in the 5′ to 3′ direction, a minimum CRISPR repeat sequence and a spacer sequence.

The sgRNA of the present invention may comprise an optimized backbone, e.g. as disclosed in Dang et al. (2015), Genome Biology, 16:280, hereby incorporated by reference. Optimizing in this context may e.g. relate to the deletion the 4×T sequence termination signal comprised in the wild type sgRNA sequence that may cause premature termination (see in this context Gao et al. 2017, Mol Ther Nucleic Acids, 10:36-44). An example for such an optimized backbone is further shown in SEQ ID NO: 29. Thus, in one embodiment, a spacer disclosed herein is combined with an optimized sgRNA backbone, e.g. SEQ ID NO: 29, preferably the spacer is upstream of the optimized sgRNA backbone sequence, i.e. the spacer sequence is 5′ of the sgRNA backbone sequence.

For example, the sgRNA may comprise or consist of a sequence of any one of SEQ ID No. 25-28 and/or 29. Likewise the gRNA may comprise a sequence as shown in SEQ ID No. 25 and 29. The gRNA can also comprise a sequence as shown in SEQ ID No. 26 and 29, 27 and 29 or 28 and 29.

By way of illustration, guide RNAs used herein or other smaller RNAs can be readily synthesized by chemical means, as illustrated below and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together.

“Sequence of interest” as used herein relates to a nucleotide sequence, preferably of the genome of a target cell that is to be excised, i.e. removed, from a gene to restore the reading frame or to repair any other type of mutation that renders the gene non-functional. One example of a gene that is affected by a frameshift leading to a truncated and non-functional protein is dystrophin. Dystrophin is a very large protein comprising many exons. Deletion of one exon may lead to a frameshift mutation. As outlined herein, a prominent example is the deletion of exon 52 of dystrophin, which could be treated by deletion of exon 51 of dystrophin. Accordingly, the sequence of interest may be exon 51 of the dystrophin gene. Preferably, the exon 51 of the dystrophin gene has a nucleic acid sequence as depicted in SEQ ID NO: 23 or 24 or a nucleic acid sequence having at least 60%, 70%, 80%, 85%, 90%, 95%, 98%, 99% or 100% sequence identity to a sequence as shown in SEQ ID 23 or 24.

Within the context of the invention, the first vector comprises a first gRNA and the second vector comprises a second gRNA. Both gRNAs are designed to excise a sequence of interest such as an exon of a gene, preferably exon 51 of dystrophin. Exon 51 of human dystrophin that may be excised by the vector system of the invention may have the nucleic acid sequence depicted in SEQ ID NO: 23. Exon 51 of porcine dystrophin that may be excised by the vector system of the invention may have the nucleic acid sequence depicted in SEQ ID NO: 24. Accordingly, at least two different sgRNAs have to be used to achieve excision of the sequence of interest—one comprised in the first vector and one comprised in the second vector. In other words, two sgRNA designed for the excision of the sequence of interest constitute a sgRNA pair. One sgRNA of the sgRNA pair may comprise a spacer complementary to the 5′ end of the sequence of interest, wherein the other sgRNA of the sgRNA pair may comprise a spacer complementary to the 3′ end of the sequence of interest. Accordingly, for excision of exon 51 of human dystrophin, the first gRNA may comprise a nucleic acid sequence as set forth in any of SEQ ID NOs: 26 and/or the second gRNA may comprise a nucleic acid sequence as set forth in any of SEQ ID NOs: 28 or vice versa. Accordingly, for excision of exon 51 of porcine dystrophin, the first gRNA may comprise a nucleic acid sequence as set forth in any of SEQ ID NOs: 25 and/or the second gRNA may comprise a nucleic acid sequence as set forth in any of SEQ ID NOs: 27 or vice versa.

Deletion of exon 51 of the dystrophin gene may restore the reading frame of the dystrophin gene and thereby enables the translation of a truncated but functional dystrophin.

The endonuclease modifies the genome of the target cell, preferably the DNA comprised in the target cell. In case, the target cell is a eukaryotic cell, the genome is localized in the nucleus. To support the function of the nuclease, the endonuclease can be fused to a nuclear localization signal (NLS) that directs the endonuclease to the nucleus. FIG. 5C shows possible positions for the NLS. One, two, three, four, five or more NLS may be N-terminal of the first fragment of the endonuclease. One, two, three, four, five or more NLS may be C-terminal of the second fragment of the endonuclease. NLS are known to a person skilled in the art. Examples for an NLS include, but are not limited to, SV40-NLS (SEQ ID NO: 15), 2×SV40-NLS (SEQ ID NO: 16) nucleoplasmin (SEQ ID NO: 17), EGL-13 (SEQ ID NO: 18), c-Myc (SEQ ID NO: 19) or TUS-protein (SEQ ID NO: 20). Preferably, the NLS is SV40-NLS. Preferably, the NLS is 2×SV40-NLS. Accordingly, the nucleic add of the first and/or the second vector can further comprise: (iv) a nuclear localization signal, preferably with the sequence selected from the list consisting of SEQ ID NOs: 15-20 or an amino acid sequence having at least 60%, 70%, 80%, 85%, 90%, 95%, 98%, 99% or 100% sequence identity to a sequence selected from the list consisting of SEQ ID NOs: 15-20. Preferably, the nuclear localization signal comprises or consists of an amino acid sequence having at least 60%, 70%, 80%, 85%, 90%, 95%, 98%, 99% or 100% sequence identity to SEQ ID NO: 15. Preferably, the nuclear localization signal comprises or consists of an amino acid sequence having at least 60%, 70%, 80%, 85% 90%, 95%, 98%, 99% 01100% sequence identity to SEQ ID NO: 16.

The elements encoded by the vector system of the invention may be operatively coupled to the promoter. An exemplary organization of the vector system of the invention is shown in FIG. 5C. In general, the first fragment of the nuclease and the first fragment of the intein of the first vector are fused and the second fragment of the intein and the second fragment of the nuclease of the second vector are fused. These fusion proteins may be each coupled operatively coupled to a promoter. In one embodiment, both fusion proteins have the same promoter. In one embodiment, the fusion proteins have different promoters. The first and the second sgRNA may be expressed independently from the nuclease fragments and the intein fragments. Accordingly, sgRNA may be operatively coupled to a (different) promoter. While the promoter for the first and the second fragment of the nuclease and the intein preferably is suitable for expression of proteins, the promoter of the sgRNA preferably is suitable for the expression of nucleic acids, preferably RNA. The promoter may be any one of U6 (SEQ ID NO: 21) or CBH (SEQ ID NO: 22). Preferably, the promoter for the sgRNA is U6, H1 or 7SK, more preferably U6. Preferably, the promoter for the first fragment of the endonuclease/first fragment of the intein fusion protein is CBH. Preferably, the promoter for the second fragment of the intein/second fragment of the endonuclease fusion protein is CBH. The promoter for the first and the second fragment of the nuclease and the intein preferably may be tissue-specific. E.g., if the vector system of the invention is used for therapy of a disease related to muscles such as DMD, a muscle-specific promoter may be used. The same might apply to a treatment of a disease of the heart, where a heart-specific promoter, e.g. the cardiac troponin (TnT) promoter, e.g. as described in Werfel et al. 2014, Cardiovascular Research, 104:15-23 or WO 2018/050783, the promoter sequences are incorporated hereby by reference, may be used. Tissue-specific promoters include, but are not limited to, B29 promoter (B cells, Promoter ID CD79B_1), CD14 promoter (monocytic cells, Promoter ID CD14_1 or CD14_2), CD43 promoter (leukocytes & platelets, Promoter ID SPN_1 or SPN_2), CD45 promoter (hematopoietic cells, Promoter ID PTPRC_1), CD68 promoter (macrophages, Promoter ID CD68_1), desmin promoter (muscle, Promoter ID DES_1), endoglin promoter (endothelial cells, Promoter ID ENG_1), fibronectin promoter (differentiating cells, healing tissues, Promoter ID FN1_1), Flt-1 promoter (endothelial cells, Promoter ID FLT1_1 or FLT1_2), GFAP promoter (astrocytes, Promoter ID GFAP_1, GFAP_2 or GFAP_3), GPIIb promoter (megakaryocytes, Promoter ID ITGA2B_1 or ITGA2B_2), ICAM-2 promoter (endothelial cells, Promoter ID ICAM2_1, ICAM2_2 or ICAM2_3), Mb promoter (muscle, Promoter ID MB_1), NphsI promoter (podocytes, Promoter ID NPHS1_1), SP-B promoter (lung, Promoter ID SFTPB_1 or SFTPB_2), SYN1 promoter (neurons, Promoter ID SYN1_1) or WASP promoter (hematopoietic cells, Promoter ID WAS_1 or WAS_2). Promoter IDs relate to entries in the Eukaryotic Promoter Database (EPD), preferably in the version of 18 Jul. 2019. The promoter references are hereby incorporated by reference from the EPD database.

Medical treatments may require the administration of the active ingredient. Accordingly, the method of the vector system for use of the present invention (i.e. in a method of treatment) may further comprise administering to the subject the first vector; and administering to the subject the second vector. The method may further comprise excising the sequence of interest.

The splitting the nuclease in two different vectors increases the safety. Until both, the first vector and the second vector, are brought in contact or are both administered to the patient, no functional endonuclease can be generated or—expressed differently—only the first and the second vector of the vector system of the invention can combine together to a functional endonuclease. The first and the second vector of the vector system of the present invention may be administered to the patient simultaneously or sequentially. Both, the first vector or the second vector could be administered first and the other one second. If the first and the second vector are administered to the patient sequentially, the time delay between the administration of the first and the second vector or the second and the first vector may be at least 5 minutes, at least 10 minutes, at least 15 minutes, at least 30 minutes, of at least 1 hour, at least 2 hours, at least 3 hours, at least 4 hours, at least 8 hours, at least 12 hours, at least 24 hours, at least 48 hours, at least 72 hours, at least 1 week or at least 2 weeks. The time delay further allows monitoring whether a patient shows any adverse reactions before administering the other vector.

The subject or patient may be a mammal, preferably a human or a pig, more preferably a human.

Several modes of administration for the vector system of the invention are known to a person skilled in the art. Exemplary modes of administration include, but are not limited to, systemic, enteral, parenteral, intravenous, intra-arterial, topical, intraperitoneal, intramuscular, intradermal, intrathecal, intravitreal, subcutaneous, transdermal and/or transmucosal administration. Pharmaceutical compositions comprising the vector system of the present invention, the first vector of the invention or the second vector of the invention may be adapted to the route of administration as described herein. Preferably, the vector system of the invention is administered parenterally.

As shown in Example 2, the first and the second vector can be administered to heart vessels (see also FIG. 15). In one embodiment, the first and the second vector can be injected into coronary artery, e.g. through an over-the-wire balloon during inflation of the balloon (e.g. blockade of the blood flow). This can allow for prolongation of contact time and more efficacious virus transduction. Alternatively, a retrograde approach may be used. Exemplarily, the administration of the first and the second vector might include the insertion of a catheter, such as a Swan-Ganz catheter, into the coronary vein, preferably accompanying the coronary artery. After balloon inflation selectively in the vein, the blood flow may be reversed by gentle increase of blood pressure and the virus solution may be injected, preferably for about 1 to 10 min, preferably for about 5 min. The accompanying vessel may be occluded by balloon inflation simultaneously, preferably in order to increase contact time and to maximize efficacy. Consequently, the first and the second vectors may be administered intravenously or intra-arterially into a vein or artery of the heart, preferably a coronary artery. The administration may comprise the use of an inflatable balloon. The administration of the first and the second vector can be done through an over-the-wire balloon.

The present invention further relates to a vector system as defined herein. Accordingly, the present invention further relates to a vector system comprising (a) a first vector comprising a nucleic acid sequence encoding: (i) a first fragment of an endonuclease, (ii) a first fragment of an intein, and (ii) a first guide RNA (gRNA); and (b) a second vector comprising a nucleic acid sequence encoding: (i) a second fragment of the endonuclease, (ii) a second fragment of the intein, and (ii) a second guide RNA (gRNA); wherein the first gRNA binds to a region, which is located 5′ to a sequence of interest comprised in a nucleic acid sequence in the genome of a target cell, wherein the second gRNA binds to a region located 3′ to a sequence of interest comprised in the nucleic acid sequence in the genome of a target cell; wherein the first fragment and the second fragment of the intein are capable of associating into a functional intein, wherein the functional intein is capable of ligating the first and the second fragment of the endonuclease to form a functional endonuclease; wherein the functional endonuclease is capable of excising the sequence of interest.

The present invention further relates to a first vector as defined herein. Accordingly, the present invention relates to a first vector comprising a nucleic acid sequence encoding: (i) a first fragment of an endonuclease, (ii) a first fragment of an intein, and (ii) a first guide RNA (gRNA).

The present invention further relates to a second vector as defined herein. Accordingly, the present invention relates to a second vector comprising a nucleic acid sequence encoding: (i) a second fragment of an endonuclease, (ii) a second fragment of an intein, and (ii) a first guide RNA (gRNA).

The present invention further relates to a combination of the first vector and the second vector of the invention.

The present invention further relates to a pharmaceutical composition comprising the vector system of the invention or comprising the combination of the invention. Such a pharmaceutical composition may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers including excipients and auxiliaries that facilitate processing of the active compound or combination into preparations that can be used pharmaceutically. Proper formulation is dependent upon the route of administration chosen.

For injection, the agents disclosed herein may be formulated in aqueous solutions, for instance in physiologically compatible buffers such as Hanks's solution, Ringer's solution, or physiological saline buffer. For transmucosal administration, penetrants appropriate to the barrier to be permeated are used in the formulation. Such penetrants are generally known in the art.

The vector system or combination of the invention may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compound or combination in water-soluble form. Additionally, a suspension of the active compound or combination may be prepared as an appropriate oily injection suspension. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran.

The pharmaceutical compositions also may include suitable solid or gel phase carriers or excipients. Examples of such carriers or excipients include but are not limited to calcium carbonate, calcium phosphate, various sugars, starches, cellulose derivatives, gelatine, and polymers such as polyethylene glycols.

Pharmaceutical compositions suitable for use in the context of the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art.

The present invention further relates to a general method of excising a sequence of interest making use the vector system of the invention. Accordingly, the present invention relates to a method for excising a sequence of interest from the genome, preferably DNA, of a subject, comprising the administration of the vector system of the invention, the combination of the invention or the pharmaceutical composition of the invention and thereby excising the sequence of interest from the genome, preferably DNA, of a subject.

The present invention is not intended to be used to modify the human germline. Accordingly, in one embodiment of the vector system, the first vector, the second vector, the combination of the first and the second vector, the pharmaceutical composition and the vector system for use of the invention does not modify the human germline.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, or and “all or any other combination of the elements connected by said term”.

The term “less than” or in turn “more than” does not include the concrete number.

For example, less than 20 means less than the number indicated. Similarly, more than or greater than means more than or greater than the indicated number, e.g. more than 80% means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

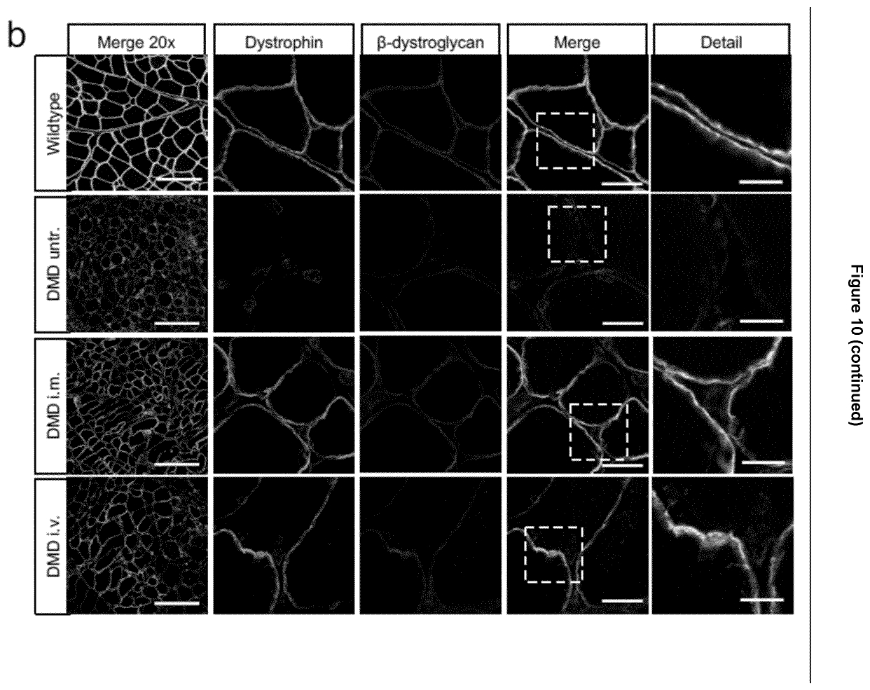

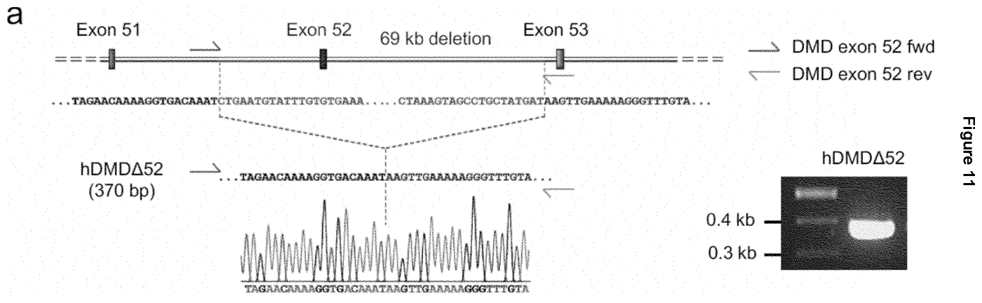

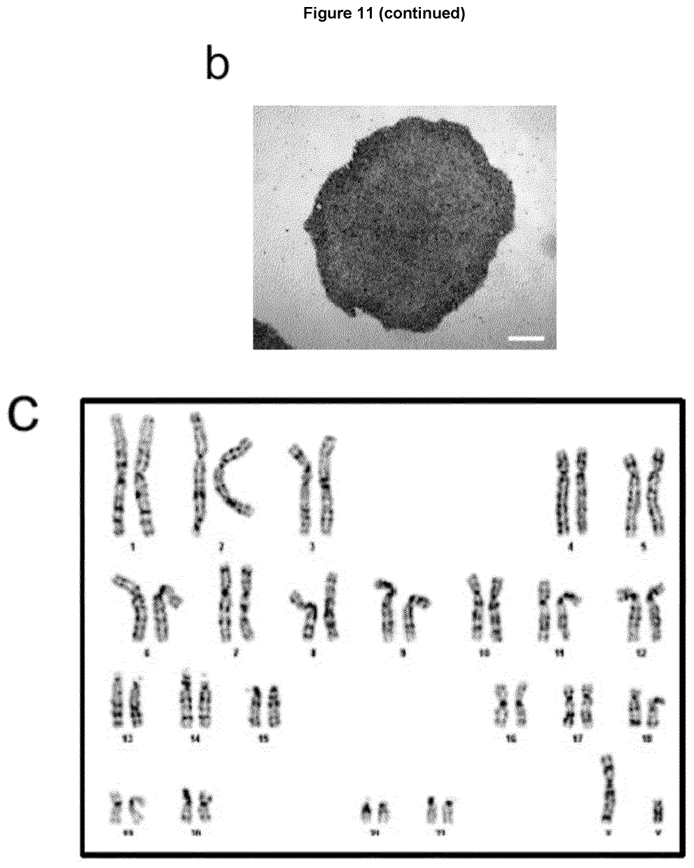

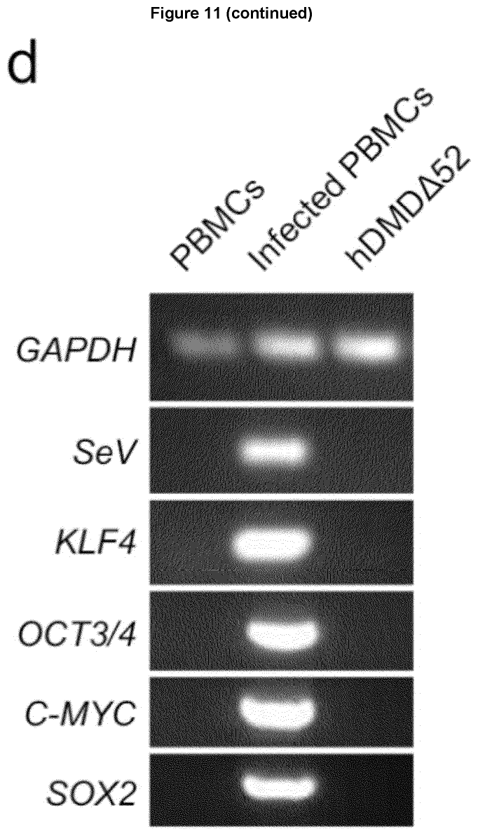

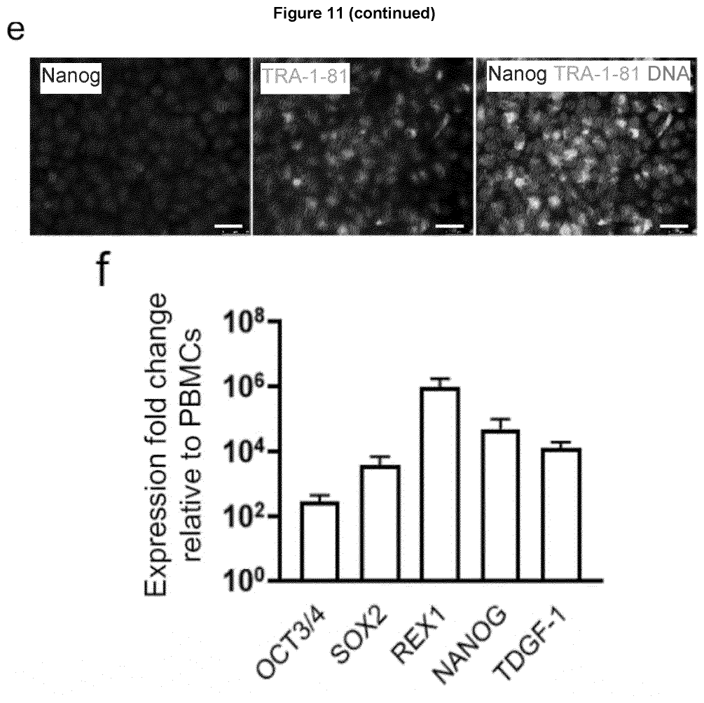

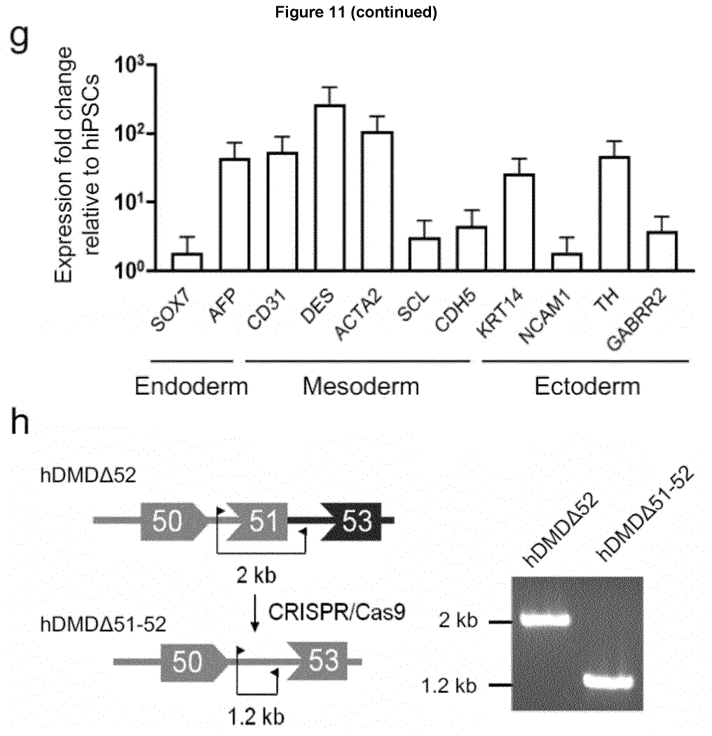

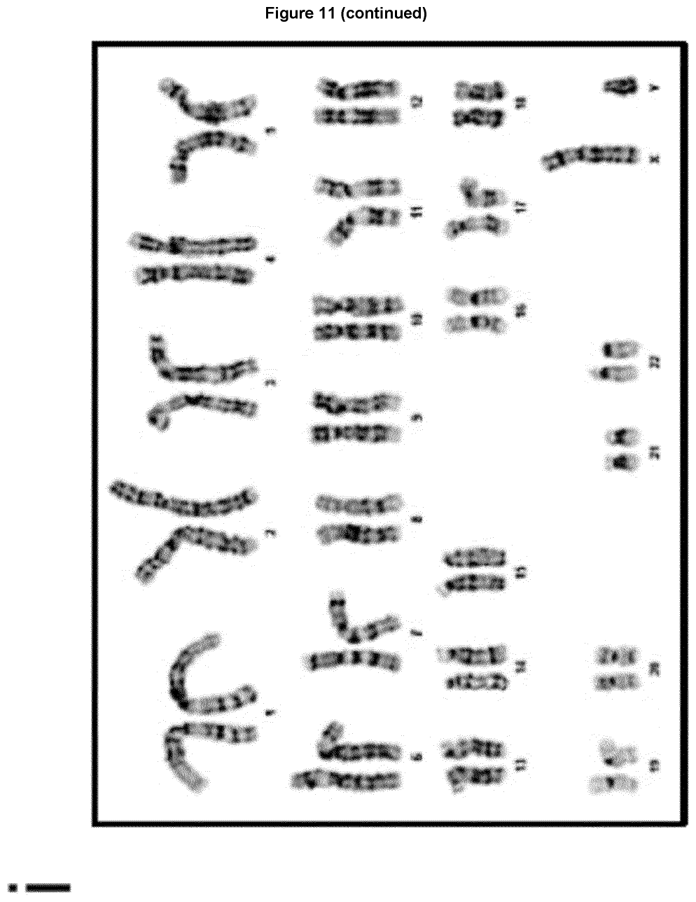

SEQUENCE LISTING
The following sequences are disclosed herein:

| SEQ ID | | |
|---|---|---|
| NO | Description | Sequence |
| 1 | Cas9 from S. | MDKKYSIGLDIGTNSVGWAVITDEYKVPSKKFKVLGNTDRHSIK |
| | pyogenes | KNLIGALLFDSGETAEATRLKRTARRRYTRRKNRICYLQEIFSNE |
| | | MAKVDDSFFHRLEESFLVEEDKKHERHPIFGNIVDEVAYHEKYP |
| | | TIYHLRKKLVDSTDKADLRLIYLALAHMIKFRGHFLIEGDLNPDNS |
| | | DVDKLFIQLVQTYNQLFEENPINASGVDAKAILSARLSKSRRLEN |
| | | LIAQLPGEKKNGLFGNLIALSLGLTPNFKSNFDLAEDAKLQLSKD |
| | | TYDDDLDNLLAQIGDQYADLFLAAKNLSDAILLSDILRVNTEITKA |
| | | PLSASMIKRYDEHHQDLTLLKALVRQQLPEKYKEIFFDQSKNGY |
| | | AGYIDGGASQEEFYKFIKPILEKMDGTEELLVKLNREDLLRKQR |
| | | TFDNGSIPHQIHLGELHAILRRQEDFYPFLKDNREKIEKILTFRIP |
| | | YYVGPLARGNSRFAWMTRKSEETITPWNFEEWDKGASAQSFI |
| | | ERMTNFDKNLPNEKVLPKHSLLYEYFTVYNELTKVKYVTEGMR |
| | | KPAFLSGEQKKAIVDLLFKTNRKVTVKQLKEDYFKKIECFDSVEI |
| | | SGVEDRFNASLGTYHDLLKIIKDKDFLDNEENEDILEDIVLTLTLF |
| | | EDREMIEERLKTYAHLFDDKVMKQLKRRRYTGWGRLSRKLING |
| | | IRDKQSGKTILDFLKSDGFANRNFMQLIHDDSLTFKEDIQKAQVS |
| | | GQGDSLHEHIANLAGSPAIKKGILQTVKWDELVKVMGRHKPEN |
| | | IVIEMARENQTTQKGQKNSRERMKRIEEGIKELGSQILKEHPVE |
| | | NTQLQNEKLYLYYLQNGRDMYVDQELDINRLSDYDVDHIVPQS |
| | | FLKDDSIDNKVLTRSDKNRGKSDNVPSEEWKKMKNYWRQLLN |
| | | AKLITQRKFDNLTKAERGGLSELDKAGFIKRQLVETRQITKHVA |
| | | QILDSRMNTKYDENDKLIREVKVITLKSKLVSDFRKDFQFYKVRE |
| | | INNYHHAHDAYLNAVVGTALIKKYPKLESEFVYGDYKVYDVRKM |
| | | IAKSEQEIGKATAKYFFYSNIMNFFKTEITLANGEIRKRPLIETNG |
| | | ETGEIVWDKGRDFATVRKVLSMPQVNIVKKTEVQTGGFSKESIL |
| | | PKRNSDKLIARKKDWDPKKYGGFDSPTVAYSVLWAKVEKGKS |
| | | KKLKSVKELLGITIMERSSFEKNPIDFLEAKGYKEVKKDLIIKLPK |
| | | YSLFELENGRKRMLASAGELQKGNELALPSKYVNFLYLASHYE |
| | | KLKGSPEDNEQKQLFVEQHKHYLDEIIEQISEFSKRVILADANLD |
| | | KVLSAYNKHRDKPIREQAENIIHLFTLTNLGAPAAFKYFDTTIDRK |
| | | RYTSTKEVLDATLIHQSITGLYETRIDLSQLGGD |
| 2 | N-split-Cas9-v1 | MDKKYSIGLDIGTNSVGWAVITDEYKVPSKKFKVLGNTDRHSIK |
| | (split between 573 | KNLIGALLFDSGETAEATRLKRTARRRYTRRKNRICYLQEIFSNE |
| | and 574) | MAKVDDSFFHRLEESFLVEEDKKHERHPIFGNIVDEVAYHEKYP |
| | | TIYHLRKKLVDSTDKADLRLIYLALAHMIKFRGHFLIEGDLNPDNS |
| | | DVDKLFIQLVQTYNQLFEENPINASGVDAKAILSARLSKSRRLEN |
| | | LIAQLPGEKKNGLFGNLIALSLGLTPNFKSNFDLAEDAKLQLSKD |
| | | TYDDDLDNLLAQIGDQYADLFLAAKNLSDAILLSDILRVNTEITKA |
| | | PLSASMIKRYDEHHQDLTLLKALVRQQLPEKYKEIFFDQSKNGY |
| | | AGYIDGGASQEEFYKFIKPILEKMDGTEELLVKLNREDLLRKQR |
| | | TFDNGSIPHQIHLGELHAILRRQEDFYPFLKDNREKIEKILTFRIP |
| | | YYVGPLARGNSRFAWMTRKSEETITPWNFEEWDKGASAQSFI |
| | | ERMTNFDKNLPNEKVLPKHSLLYEYFTVYNELTKVKYVTEGMR |
| | | KPAFLSGEQKKAIVDLLFKTNRKVTVKQLKEDYFKKIE |
| 3 | N-split-Cas9-v2 | MDKKYSIGLDIGTNSVGWAVITDEYKVPSKKFKVLGNTDRHSIK |
| | (split between 637 | KNLIGALLFDSGETAEATRLKRTARRRYTRRKNRICYLQEIFSNE |
| | and 638) | MAKVDDSFFHRLEESFLVEEDKKHERHPIFGNIVDEVAYHEKYP |
| | | TIYHLRKKLVDSTDKADLRLIYLALAHMIKFRGHFLIEGDLNPDNS |
| | | DVDKLFIQLVQTYNQLFEENPINASGVDAKAILSARLSKSRRLEN |
| | | LIAQLPGEKKNGLFGNLIALSLGLTPNFKSNFDLAEDAKLQLSKD |
| | | TYDDDLDNLLAQIGDQYADLFLAAKNLSDAILLSDILRVNTEITKA |
| | | PLSASMIKRYDEHHQDLTLLKALVRQQLPEKYKEIFFDQSKNGY |
| | | AGYIDGGASQEEFYKFIKPILEKMDGTEELLVKLNREDLLRKQR |
| | | TFDNGSIPHQIHLGELHAILRRQEDFYPFLKDNREKIEKILTFRIP |
| | | YYVGPLARGNSRFAWMTRKSEETITPWNFEEWDKGASAQSFI |
| | | ERMTNFDKNLPNEKVLPKHSLLYEYFTVYNELTKVKYVTEGMR |
| | | KPAFLSGEQKKAIVDLLFKTNRKVTVKQLKEDYFKKIECFDSVEI |
| | | SGVEDRFNASLGTYHDLLKIIKDKDFLDNEENEDILEDIVLTLTLF |
| | | EDREMIEERLK |
| 4 | C-split-Cas9-v1 | CFDSVEISGVEDRFNASLGTYHDLLKIIKDKDFLDNEENEDILEDI |
| | (split between 573 | VLTLTLFEDREMIEERLKTYAHLFDDKVMKQLKRRRYTGWGRL |
| | and 574) | SRKLINGIRDKQSGKTILDFLKSDGFANRNFMQLIHDDSLTFKED |
| | | IQKAQVSGQGDSLHEHIANLAGSPAIKKGILQTVKVVDELVKVM |
| | | GRHKPENIVIEMARENQTTQKGQKNSRERMKRIEEGIKELGSQI |
| | | LKEHPVENTQLQNEKLYLYYLQNGRDMYVDQELDINRLSDYDV |
| | | DHIVPQSFLKDDSIDNKVLTRSDKNRGKSDNVPSEEWKKMKN |
| | | YWRQLLNAKLITQRKFDNLTKAERGGLSELDKAGFIKRQLVETR |
| | | QITKHVAQILDSRMNTKYDENDKLIREVKVITLKSKLVSDFRKDF |
| | | QFYKVREINNYHHAHDAYLNAWGTALIKKYPKLESEFVYGDYK |
| | | VYDVRKMIAKSEQEIGKATAKYFFYSNIMNFFKTEITLANGEIRK |
| | | RPLIETNGETGEIVWDKGRDFATVRKVLSMPQVNIVKKTEVQTG |
| | | GFSKESILPKRNSDKLIARKKDWDPKKYGGFDSPTVAYSVLWA |
| | | KVEKGKSKKLKSVKELLGITIMERSSFEKNPIDFLEAKGYKEVKK |
| | | DLIIKLPKYSLFELENGRKRMLASAGELQKGNELALPSKYVNFLY |
| | | LASHYEKLKGSPEDNEQKQLFVEQHKHYLDEIIEQISEFSKRVIL |
| | | ADANLDKVLSAYNKHRDKPIREQAENIIHLFTLTNLGAPAAFKYF |
| | | DTTIDRKRYTSTKEVLDATLIHQSITGLYETRIDLSQLGGD |
| 5 | C-split-Cas9-v2 | TYAHLFDDKVMKQLKRRRYTGWGRLSRKLINGIRDKQSGKTIL |
| | (split between 637 | DFLKSDGFANRNFMQLIHDDSLTFKEDIQKAQVSGQGDSLHEHI |
| | and 638) | ANLAGSPAIKKGILQTVKWDELVKVMGRHKPENIVIEMARENQ |
| | | TTQKGQKNSRERMKRIEEGIKELGSQILKEHPVENTQLQNEKLY |
| | | LYYLQNGRDMYVDQELDINRLSDYDVDHIVPQSFLKDDSIDNKV |
| | | LTRSDKNRGKSDNVPSEEWKKMKNYWRQLLNAKLITQRKFDN |
| | | LTKAERGGLSELDKAGFIKRQLVETRQITKHVAQILDSRMNTKY |
| | | DENDKLIREVKVITLKSKLVSDFRKDFQFYKVREINNYHHAHDA |
| | | YLNAWGTALIKKYPKLESEFVYGDYKVYDVRKMIAKSEQEIGK |
| | | ATAKYFFYSNIMNFFKTEITLANGEIRKRPLIETNGETGEIVWDK |
| | | GRDFATVRKVLSMPQVNIVKKTEVQTGGFSKESILPKRNSDKLI |
| | | ARKKDWDPKKYGGFDSPTVAYSVLWAKVEKGKSKKLKSVKEL |
| | | LGITIMERSSFEKNPIDFLEAKGYKEVKKDLIIKLPKYSLFELENG |
| | | RKRMLASAGELQKGNELALPSKYVNFLYLASHYEKLKGSPEDN |
| | | EQKQLFVEQHKHYLDEIIEQISEFSKRVILADANLDKVLSAYNKH |
| | | RDKPIREQAENIIHLFTLTNLGAPAAFKYFDTTIDRKRYTSTKEVL |
| | | DATLIHQSITGLYETRIDLSQLGGD |
| 6 | Intein from N. | CLSYETEILTVEYGLLPIGKIVEKRIECTVYSVDNNGNIYTQPVAQ |
| | punctiforme | WHDRGEQEVFEYCLEDGSLIRATKDHKFMTVDGQMLPIDEIFE |
| | | RELDLMRVDNLPNMIKIATRKYLGKQNVYDIGVERDHNFALKNG |
| | | FIASN |
| 7 | NrdJ-1 Intein | CLVGSSEIITRNYGKTTIKEWEIFDNDKNIQVLAFNTHTDNIEWA |
| | | PIKAAQLTRPNAELVELEIDTLHGVKTIRCTPDHPVYTKNRGYVR |
| | | ADELTDDDELWAIMEAKTYIGKLKSRKIVSNEDTYDIQTSTHNF |
| | | FANDILVHN |
| 8 | Gp41-1 Intein | CLDLKTQVQTPQGMKEISNIQVGDLVLSNTGYNEVLNVFPKSKK |
| | | KSYKITLEDGKEIICSEEHLFPTQTGEMNISGGLKEGMCLYVKE |
| | | MMLKKILKIEELDERELIDIEVSGNHLFYANDILTHN |
| 9 | N-terminal | CLSYETEILTVEYGLLPIGKIVEKRIECTVYSVDNNGNIYTQPVAQ |
| | splicing region of | WHDRGEQEVFEYCLEDGSLIRATKDHKFMTVDGQMLPIDEIFE |
| | N. punctiforme | RELDLMRVDNLPN |
| 10 | N-terminal | CLVGSSEIITRNYGKTTIKEWEIFDNDKNIQVLAFNTHTDNIEWA |
| | splicing region of | PIKAAQLTRPNAELVELEIDTLHGVKTIRCTPDHPVYTKNRGYVR |
| | NrdJ-1 | ADELTDDDELWAI |
| 11 | N-terminal | CLDLKTQVQTPQGMKEISNIQVGDLVLSNTGYNEVLNVFPKSKK |
| | splicing region of | KSYKITLEDGKEIICSEEHLFPTQTGEMNISGGLKEGMCLYVKE |
| | gp41-1 | |
| 12 | C-terminal | MIKIATRKYLGKQNVYDIGVERDHNFALKNGFIASN |
| | splicing region of | |
| | N. punctiforme | |
| 13 | C-terminal | MEAKTYIGKLKSRKIVSNEDTYDIQTSTHNFFANDILVHN |
| | splicing region of | |
| | NrdJ-1 | |
| 14 | C-terminal | MMLKKILKIEELDERELIDIEVSGNHLFYANDILTHN |
| | splicing region of | |
| | g41-1 | |
| 15 | SV40 NLS | PKKKRKV |
| 16 | 2xSV40 NLS | PKKKRKVEDPKKKRKVD |
| 17 | Nucleoplasmin | AVKRPAATKKAGQAKKKKLD |
| | NLS | |
| 18 | ELG-13 NLS | MSRRRKANPTKLSENAKKLAKEVEN |
| 19 | c-Myc NLS | PAAKRVKLD |
| 20 | TUS protein NLS | KLKIKRPVK |
| 21 | U6 promoter | AAGGTCGGGCAGGAAGAGGGCCTATTTCCCATGATTCCTTC |
| | | ATATTTGCATATACGATACAAGGCTGTTAGAGAGATAATTAG |
| | | AATTAATTTGACTGTAAACACAAAGATATTAGTACAAAATACG |
| | | TGACGTAGAAAGTAATAATTTCTTGGGTAGTTTGCAGTTTTAA |
| | | AATTATGTTTTAAAATGGACTATCATATGCTTACCGTAACTTG |
| | | AAAGTATTTCGATTTCTTGGCTTTATATATCTTGTGGAAAGGA |
| | | CG |
| 22 | CHB promoter | CGTTACATAACTTACGGTAAATGGCCCGCCTGGCTGACCGC |
| | (composed of | CCAACGACCCCCGCCCATTGACGTCAATAGTAACGCCAATA |
| | CMV enhancer, | GGGACTTTCCATTGACGTCAATGGGTGGAGTATTTACGGTAA |
| | chicken actin core | ACTGCCCACTTGGCAGTACATCAAGTGTATCATATGCCAAGT |
| | promoter, 5′- | ACGCCCCCTATTGACGTCAATGACGGTAAATGGCCCGCCTG |
| | splice site from | GCATTGTGCCCAGTACATGACCTTATGGGACTTTCCTACTTG |
| | chicken actin and | GCAGTACATCTACGTATTAGTCATCGCTATTACCATGGTCGA |
| | 3′-splice site from | GGTGAGCCCCACGTTCTGCTTCACTCTCCCCATCTCCCCCC |
| | minute virus from | CCTCCCCACCCCCAATTTTGTATTTATTTATTTTTTAATTATTT |
| | mice (MVM).) | TGTGCAGCGATGGGGGCGGGGGGGGGGGGGGGGCGCGC |
| | See e.g. Gray et | GCCAGGCGGGGCGGGGCGGGGCGAGGGGCGGGGCGGGG |
| | al. (2011). Hum | CGAGGCGGAGAGGTGCGGCGGCAGCCAATCAGAGCGGCG |
| | Gene Ther. 2011 | CGCTCCGAAAGTTTCCTTTTATGGCGAGGCGGCGGCGGCG |
| | Sep; 22(9): 1143- | GCGGCCCTATAAAAAGCGAAGCGCGCGGCGGGCGGGAGTC |
| | 1153, hereby | GCTGCGACGCTGCCTTCGCCCCGTGCCCCGCTCCGCCGCC |
| | incorporated by | GCCTCGCGCCGCCCGCCCCGGCTCTGACTGACCGCGTTAC |
| | reference. | TCCCACAGGTGAGCGGGCGGGACGGCCCTTCTCCTCCGGG |
| | | CTGTAATTAGCTGAGCAAGAGGTAAGGGTTTAAGGGATGGT |
| | | TGGTTGGTGGGGTATTAATGTTTAATTACCTGGAGCACCTGC |
| | | CTGAAATCACTTTTTTTCAGGTTGG |
| 23 | Human exon 51 of | AACAGGATTGTCTACCAGACATTTTAATTCTAGTACTATGCAT |
| | DMD | CTTAACCATTACCATAGGCTGACTTACTCTACAGTGTCCAAC |
| | | ACTATTCATATTAAGATTTATTTAATGACTTTGAAACAGTATTT |
| | | CATGTCTAAATAGAAAAACTACTAACTCGCATTTTTAAGAAAA |
| | | TATTGTATCTTGGTTTTTCTTCACTGCTGGCCAGTTTACTAAC |
| | | AATCTGAAATAAAAAGAAAAAAATATGATAAACTGCTCCCAGT |
| | | ATAAAATACAGAGCTAAGACAAGAACGTTTCATTGGCTTTGA |
| | | TTTCCCTAGGGTCCAGCTTCAAATTAATTTACTTCCTATTCAA |
| | | GGGAATTTTAAATCAGAAAGAAGATCTTATCCCATCTTGTTTT |
| | | GCCTTTGTTTTTTCTTGAATAAAAAAAAAATAAGTAAAATTTAT |
| | | TTCCCTGGCAAGGTCTGAAAACTTTTGTTTTCTTTACCACTTC |
| | | CACAATGTATATGATTGTTACTGAGAAGGCTTATTTAACTTAA |
| | | GTTACTTGTCCAGGCATGAGAATGAGCAAAATCGTTTTTTAA |
| | | AAAATTGTTAAATGTATATTAATGAAAAGGTTGAATCTTTTCAT |
| | | TTTCTACCATGTATTGCTAAACAAAGTATCCACATTGTTAGAA |
| | | AAAGATATATAATGTCATGAATAAGAGTTTGGCTCAAATTGTT |
| | | ACTCTTCAATTAAATTTGACTTATTGTTATTGAAATTGGCTCTT |
| | | TAGCTTGTGTTTCTAATTTTTCTTTTTCTTCTTTTTTCCTTTTTG |
| | | CAAAAACCCAAAATATTTTAGCTCCTACTCAGACTGTTACTCT |
| | | GGTGACACAACCTGTGGTTACTAAGGAAACTGCCATCTCCAA |
| | | ACTAGAAATGCCATCTTCCTTGATGTTGGAGGTACCTGCTCT |
| | | GGCAGATTTCAACCGGGCTTGGACAGAACTTACCGACTGGC |
| | | TTTCTCTGCTTGATCAAGTTATAAAATCACAGAGGGTGATGG |
| | | TGGGTGACCTTGAGGATATCAACGAGATGATCATCAAGCAG |
| | | AAGGTATGAGAAAAAATGATAAAAGTTGGCAGAAGTTTTTCT |
| | | TTAAAATGAAGATTTTCCACCAATC |
| 24 | Porcine exon 51 | GAGAGGAATTCTCTGCCAGCTATTTTTACTCTGGAACTTCAG |
| | DMD | GTCTTAACCATTACCTTGTGTTCCCTTACACCACAATGTTCAA |
| | | CACTATTAACATTAATTTAATATTTCTTAAGAGCCTTGAAATG |
| | | GTGTCTCATTATCCAAGTGGAAAAACTTTACTAGCTCTGATTT |
| | | TCCAAGAATAATATAGCTTGGGTTTTCTTTACTGCTAGCCACT |
| | | TTATTATCAGTCTGAAAGAAAAAGAAAAAAAATATATAAACTG |
| | | CTACCAATATAAAATAAAGAGCAAAGGCAAGAACATTTCATT |
| | | GGTTTTGATTTCCCTAGAGTCCACTTCCAAAGCAATTTACCT |
| | | CTTATTCAAGGAAATTGTAAGTTTGAAATAAGATCTTATCCAA |
| | | TTTTTACTTTTTTCCCCTTGAACAAAAAAAAAAGTAAAACATTC |
| | | ACCTTATCAAAGTTTGAATACTTTATATTTTTCTTTACCAGTTC |
| | | AGCAACTGTATAATTGTTATTGAGGGCTTTTTAGGTTAAATTA |
| | | ATTGCTCACCTATAGGAATAATCAAAATGGTTTTGAAAGATTG |
| | | TTAAATATGTATTAATGAAAAATTTGAATCTTTGAATTATTCTA |
| | | CCATGTAGTACTCATGTTTCTAGGAGAAAAACATGTAATGTC |
| | | ATAAAAAATACTTTGGCTCAAATTGTTGCCCTTTAATTAGTTT |
| | | TATTGTTATTGAAACTGGCTTCTTAGCTTGTGTTTCTAATTTT |
| | | CCTTTTTTCTTTTTCCCTTTTTGAAAAAAAAAATTTAGCTACTA |
| | | GTCAGACTGTTACTCTGGTGACACAACCCACAGTTACCAAG |
| | | GAAACTGCCATCTCCAAACCAGAAATGCCATCTTCCTTGCTG |
| | | TTGGAGGTACCTGCTCTGGCAGATTTCAACAGGGCTTGGAC |
| | | AGAACTTACCGACTGGCTGTCTCTGCTTGATCGAGTTATAAA |
| | | ATCACAGAGGGTGATGGTGGGTGATCTTGAAGACATCAACG |
| | | AGATGATCATCAAGCAGAAGGTATGGGGGGAATAAAGATAA |
| | | GAGCTGG |
| 25 | first spacer | GAGAGTTCCTAAGGTAGAGAG |
| | sequence pig | |
| 26 | first spacer | GATAAAGATAAGAGCTGGCAG |
| | sequence human | |
| 27 | second spacer | GTAATTTGAAGCTGGACCCTA |
| | sequence pig | |
| 28 | second spacer | GTCTAGGAGAGTAAAGTGAT |
| | seguence human | |
| 29 | sgRNA backbone | GTTTAAGAGCTATGCTGGAAACAGCATAGCAAGTTTAAATAA |
| | sequence | GGCTAGTCCGTTATCAACTTGAAAAAGTGGCACCGAGTCGG |
| | | TGCTTTTTTT |
| 30 | Sequence from | TAGAACAAAAGGTGACAATCTGAATGTATTTGTGTGAAA |
| | FIG. 11A | |
| 31 | Sequence from | CTAAAGTAGCCTGCTATGATAAGTTGAAAAAGGGTTTGTA |
| | FIG. 11A | |
| 32 | Sequence from | TAGAACAAAAGGTGACAAATAAGTTGAAAAAGGGTTTGTA |
| | FIG. 11A | |
| 33 | oligonucleotide | 5′ TAATTTGAAGCTGGACCCTA |
| 34 | oligonucleotide | 5′ GTCTAGGAGAGTAAAGTGAT |

### EXAMPLES

An even better understanding of the present invention and of its advantages will be evident from the following examples, offered for illustrative purposes only. The examples are not intended to limit the scope of the present invention in any way.

### Methods

All animal experiments were approved by the Bavarian Animal Care and Use Committee and conform to the Guide for the Care and Use of Laboratory Animals published by the US National Institutes of Health (NIH Publication No. 85-23, revised 1996, Approved Institution #A5637-01)

### DMDΔ52 Animal Generation

Animals were produced by breeding from a herd comprising heterozygous female DMD^{+/-} pigs. The breeding herd was established from a single sow with a heterozygous DMD exon 52 deletion (DMDΔ52). Exon 52 of the porcine DMD gene was deleted according to³². In brief, BAC CH242-9G11 was modified to carry a neomycin selection cassette in place of exon 52; the BAC was nucleofected into a female primary kidney cell line (PKCf) and single cell clones were generated³³. Genomic DNA was isolated from a batch of each cell clone and the copy number of the DMD exon 52 was compared to the copy numbers of two reference loci within the NANOG and the POU5F1 genes for identifying cell clones with a heterozygous DMD exon 52 deletion³⁴. A total of 258 cell clones was screened and 9 of them had one modified allele as well as an intact one (3.49% efficacy). These cell clones were used in somatic cell nuclear transfer (SCNT) to produce heterozygous DMDΔ52 carrier sows³⁵. A total of 14 SCNT was performed including DMD^{+/-} cells and within 3 litters delivered 4 offspring were proven to retain the desired DMD^{+/-} genotype. One animal was raised and inseminated with wild-type sperm. Male DMD^{Y/-} offspring were used as experimental animals and female DMD^{+/-}animals were used to expand the breeding capacity. Genotyping of the offspring was performed by PCR. In total, 73 affected DMD^{Y/-} pigs were produced by breeding, of which 28 were used in this study.

gRNA Design and Off-Target Analysis

Specific gRNAs in intron 50 and 51 (listed in below Table 2 were cloned into the N-Cas9_N-Intein_v2 and C-Intein_C-Cas9_v2 vector respectively³⁷ and transfected into a porcine or human cell line for testing editing efficacy. The gRNA combination showing highest activity has been chosen. Sequences of the spacers chosen for the sgRNAs are listed in Table 2. These spacers are then combined with the backbone sequence of e.g. SEQ ID NO. 29

TABLE 2
Sequences of spacers comprised in the sgRNAs.

| | | SEQ |
|---|---|---|
| Name | Spacer Sequence | ID NO |
| Ss_DMD_gRNA_Intron50 5′ | GAGAGTTCCTAAGGTAGAGAG | 25 |
| Ss_DMD_gRNA_Intron51 5′ | GATAAAGATAAGAGCTGGCAG | 27 |
| Hs_DMD_gRNA_Intron50 5′ | GTAATTTGAAGCTGGACCCTA | 26 |
| Hs_DMD_gRNA_Intron51 5′ | GTCTAGGAGAGTAAAGTGAT | 28 |

Potential off-targets have been predicted using the CRISPOR and the CHOPCHOP web tool⁵¹ and ranked according to their CFD and MIT score. The top five predicted off-targets for both the intron 50 and intron 51 gRNA were amplified by PCR using Q5 polymerase (NEB) with standard conditions on genomic DNA from one control pig, two intramuscular treated animals and one systemically injected pig. A total of 400 ng of genomic DNA has been used per reaction. PCR products subsequently were analyzed by deep sequencing.

### Sequencing for Off-Target Analysis

Libraries were sequenced in paired end mode with 100 bases read length in a HiSeq 1500. Data analysis was performed with the Bioconductor package CrispRVariants. For each off target region, the reference sequence shows the gRNAs and PAM sequence marked by a black rectangle and additional 5 nucleotide up- and downstream.

Position referred to the cut site and type of detected INDELs or INDEL combinations found in a dedicated read were listed at the left. Deletions were marked with ‘-’ and distinct colored symbols indicate the position of insertions. The tables show in the first line the number of sequence reads matching the reference sequence and in the following lines the number of INDELS found in each sample.

Levenshtein Distance of gRNA Around Genomic SNP/INDELs.

Genome-wide sequencing of human DMDΔ52 iPSCs and an isogenic edited DMDΔ51-52 iPSC clone was achieved using an Illumine HiSeq 1500 sequencer. The reads were sequenced in paired-end mode with a length of 100 nt. SNPs and INDELs in each sample were called using the GATK somatic SNV+INDEL pipeline⁴⁷ and filtered for SNPs/INDELs specific to the edited DMDΔ51-52 iPSC clone. In total, 769 SNPs and 88 INDELs were identified. To clarify if these variants represent off-target effects or occurred randomly during the clonal expansion of the edited DMDΔ51-52 iPSCs, a minimal Levenshtein distance analysis was performed. The two guide RNAs were aligned in a sliding window starting 25 bp upstream and ending 25 bp downstream of each variant and the alignment with the smallest difference was determined.

### Virus Preparation

Recombinant adeno-associated viruses of the serotype 9 and 6 were produced with the triple transfection method as described previously³⁸. Briefly, the packaging cell line HEK 293T was transfected with the vector Cbh-N-Cas9/CRISPR 5-1 or Cbh-C-Cas9/CRISPR 3-1, a plasmid encoding the cap sequences of AAV9 (pigs) and AAV6 (hiPSCs) and rep AAV2 sequences and the helper plasmid delta F6 (Puresyn, Pa.) using PEI Max (Polysciences). After 72 hours, cells were harvested and virus was purified by iodixanol-gradient centrifugation. The virus was further purified by a gravity flow size exclusion purification using Sepharose G100 SF resin (Sigma-Aldrich) in Econopac colums (Biorad). Virus was concentrated in PBS using Amicon Ultra-15 Centrifugal Filter Units (Merck) and stored at 4° C. Viral titer was quantified by ITR-Probe qPCR.

### AAV9-Cas9-gE51 Transduction and Follow Up

For intramuscular transduction, the right side of the animal was treated by injections of 200 μl each (100 μl of each virus subsequently) in 15 injection sites (9× thigh and 6× upper arm) at day 14 after birth. Of each Cas9-intein-half (N-Cas9 and C-Cas9), 2.5×10¹³ vg/kg bodyweight were injected in total in 5 animals. At the end of the experiment, tissue was recovered from injected and contralateral muscles and remote organs and analyzed for dystrophin expression.

For systemic application, 4 weeks old piglets were injected 2×10¹³ vg/kg for each, N-Cas9 and C-Cas9 into the ear vein (low dose) or 2×10¹⁴ vg/kg for each strain, totaling e.g. 2×10¹⁵ vg for a 5 kg piglet.

### Mortality and Termination of Experiments

In total, 73 affected DMD^{Y/-} piglets could be produced in our DMD pig breeding herd, of which 45 newborn pigs died within the first week despite intense nursing and adjuvant feeding and were excluded from further studies. 28 male DMD^{Y/-} were used in this study. Of these, 3 animals (1 i.m., 1 i.v. animal) were observed for a pre-specified period (69 days and 77 days, respectively) to investigate protein expression. Other DMD pigs were followed until physical deterioration (tachypnea, exhaustion, stridor), triggering termination of the experiment due to animal protection regulation. Alternately, DMD animals succumbed to sudden cardiac death, which occurred at rest (with video documentation in place for all i.v. treated animals) or during individual and veterinarian-accompanied transportation (2 i.m. treated DMD animals and 1 high-dose treated DMD animal), such that transportation of the last 7 DMD animals was performed after intubation under anesthesia with 1 animal was lost before measurements once intubated.

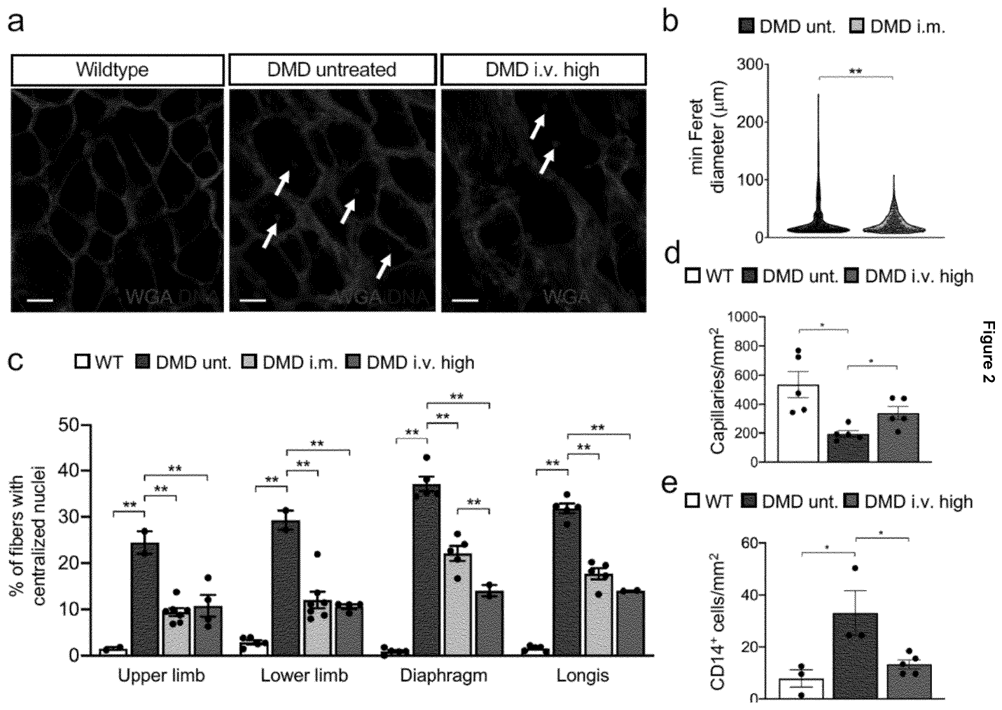

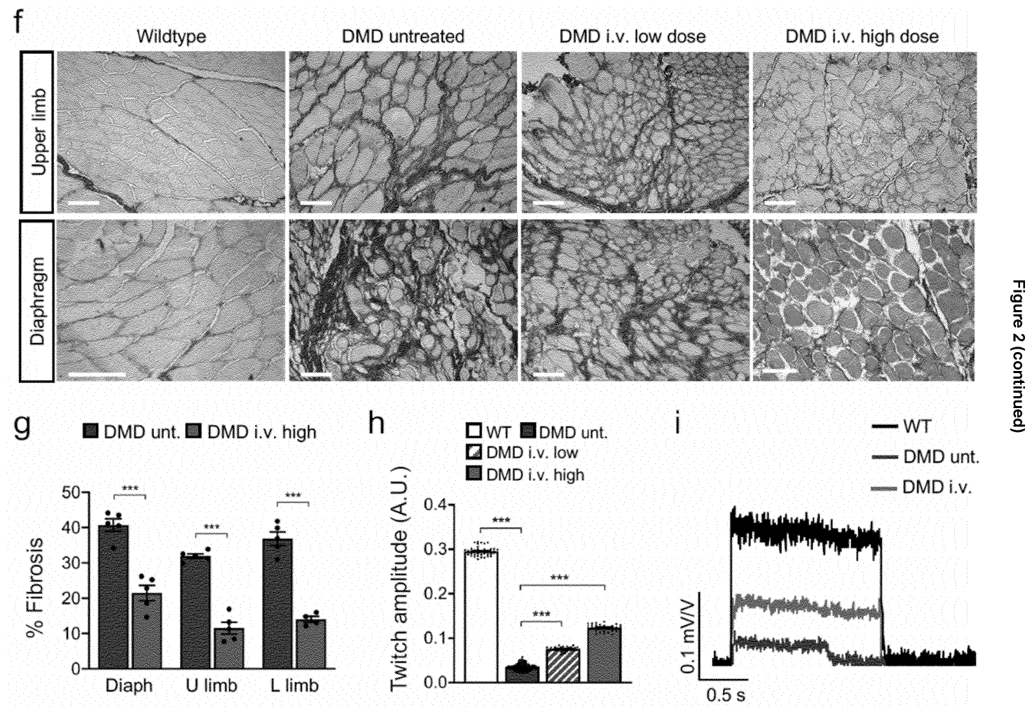

The investors investigated 4 high dose i.v. treated animals, 3 DMD animals with untreated hearts (2 i.m. and 1 untreated heart) and 3 wildtype siblings of DMD pigs by cardiac catheterization (cf. FIG. 3b-d). Electrophysiological mapping was performed in 3 high dose treated DMD animals, 2 untreated DMD hearts and 3 wildtype animals.

### Functional Behavior Measurement

Behavioral observation methods were used according to Martin, P. and Bateson, P. 1995. Measuring Behaviour: An introductory guide. Cambridge University Press. Instantaneous sampling records the state of an individual animal at predetermined time intervals. Animals were observed at every 5th minute for 24 hours. In order to ensure a reliable measurement the second before and after was involved in the evaluation but only the centisecond of the instant was graded. Blue color indicates a lying or sitting posture, orange color indicates action. Gray color indicates no sight to the animal. Continuous recording enabled the observation of total activity and resting periods in 24 hours. Two states (upright and lying posture) were recorded to the second. Frequency and duration of standing and lying postures were evaluated.

### Cardiac Catheterization and High-Resolution 3D Mapping

Pigs were anesthetized and instrumented as previously described^{39,40}. Briefly, global myocardial function was assessed by pressure-tip catheter placement in the left ventricle (for LV enddiastolic and systolic pressures, dP/dtₘₐₓ, dP/dtₘᵢₙ) at rest and rapid atrial pacing (150/min), whereas analysis of ejection fraction was performed after LV angiography in anterior-posterior position (yielding slightly smaller control values than a right anterior oblique view).

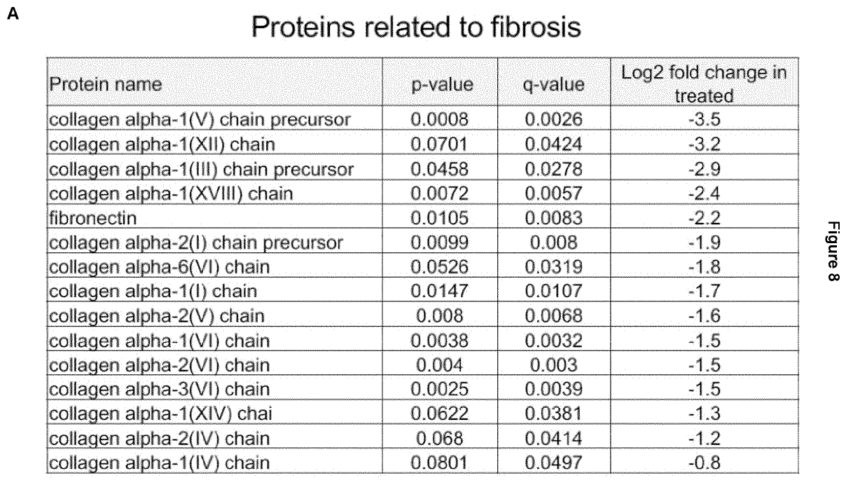

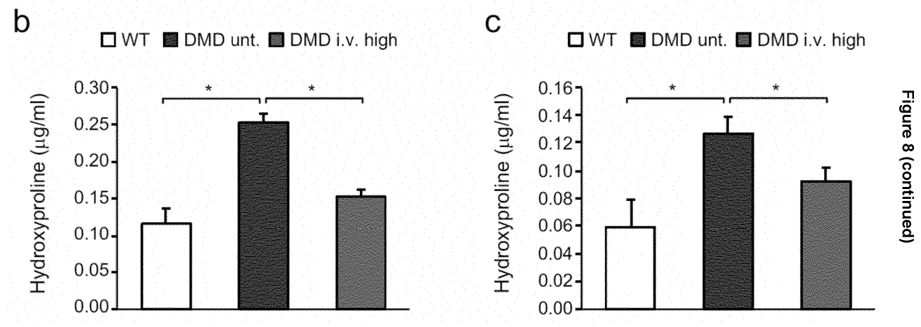

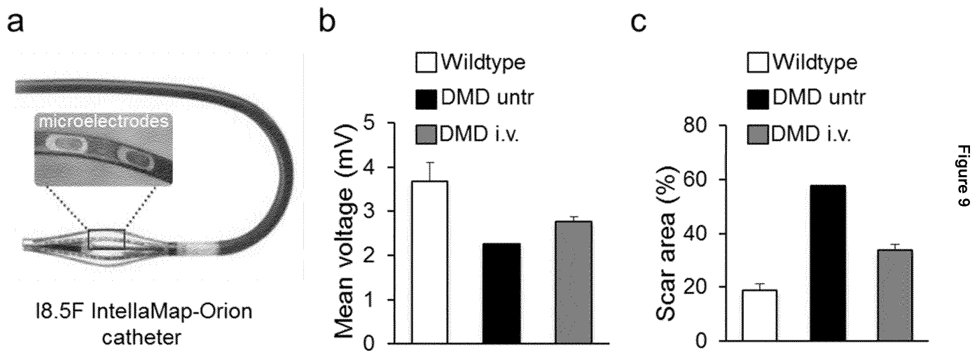

The Rhythmia mapping system was used for high-resolution 3D-mapping (Boston Scientific, Natick, Mass.), as described before⁴¹. Bipolar activation maps were created in 3 wildtype hearts, an untreated DMD heart, and 3 high-dose treated hearts (FIG. 3e). The 18.5F IntellaMap-Orion catheter (FIG. 9a) contains 64 flat microelectrodes (0.8 mm diameter) in a basket configuration with 8 splines. The basket is steerable in 2 directions and can be opened and closed to provide appropriate wall contact for detection of electrophysiological signals. Cardiac beats were automatically selected by the mapping system based on standard beat acceptance criteria: cycle length stability, 12-lead electrocardiogram morphology match, electrode location stability, and respiratory gating. If the signals did not satisfy the above criteria, the information was not included in the map. The LV surface geometry was generated by including all points recorded within 2 mm from the outermost surface of the map (defined by outermost reach of any of the electrodes in 3D space). The voltage for bipolar electrograms was derived measuring from peak to peak. The low-voltage area and endocardial scar are were defined on the bipolar voltage map as <1.3 mV and <0.3 mV, respectively. Quantitative analysis of the electroanatomical maps.

Maximal, minimal and mean bipolar electrogram voltage was calculated for each LV-map. Quantification of low voltage scar areas—defined as bipolar voltage <1.3 mV—was done using the paraView open-source, multi-platform data analysis and visualization application (Kitware, Clifton Park, N.Y., USA) (FIG. 9 b,c).

### Tibiotarsal Joint Force Measurements

The physiology rig was set up as described by Childers et al.⁴² using a bridge interface and load cell obtained from Phidgets Inc., Calgary, Canada. Anesthetized pigs were placed on the rig in dorsal recumbent position and hoof was strapped to foot pedal with maintaining a 90° angle for the coxofemoral, knee and tibiotarsal joints. Needle electrodes were placed on either side of the common peroneal nerve to stimulate tibiotarsal flexion. Isometric twitches were triggered with individual 150 V, 100 μsec pulses, tetanic contraction was obtained with 1% sec train of pulses at 50 Hz.

### Histology

Fibrosis was detected by Sirius red staining of paraffin-embedded tissues. Pictures were taken at a 20-fold magnification. Fibrosis quantity was determined from 10 independent images each with Image J Software. Dystrophin was detected in frozen tissues with antibodies directed against the C-terminus (Novocastra NCL-DYS2, Wetzlar, Germany). A CD14 antibody (Biorad MCA1218F, Munich, Germany) was used for detection of immunological cells (FIG. 2).

For DGC analysis, all images were recorded from 7 μm sections of muscle tissue frozen in isopentane chilled in liquid nitrogen using identical confocal imaging parameters (Olympus FluoView F1000).

### Hydroxyproline Assay

Hydroxyproline in porcine tissue samples was quantified via Colorimetric Hydroxyproline Assay Kit (ab222941, Abcam, Cambridge, UK). 100 mg of each sample was homogenized in 300 μl and subjected to alkaline lysis at 121° C. for 2 hours. 10 μl of the lysates was assayed as per manufacturer's protocol.

### Gel Electrophoresis, Immunoblotting and Preparation of Bands for Mass Spectrometry Analysis

Muscle tissue sample were homogenized in lysis buffer (125 mm Tris pH 8.8, 40% glycerol, 4% SDS, 0.5 mm PMSF, 100 mm DTT) using an ultrasonic device (46 kJ, Sonoplus GM3200 with BR30 cup booster, Bandelin, Berlin, Germany). Protein concentration was determined using the Pierce 660 nm Protein Assay (Thermo Fisher Scientific, Rockford, Ill., USA). SDS gel electrophoresis was performed using a 4-20% Mini-PROTEAN® TGX™ precast gel (Bio-Rad, Hercules, Calif., USA)³². After separation the gel was Coomassie stained using Roti-Blue (Carl Roth, Karlsruhe, Germany). Immunblotting was performed with a C-terminus dystrophin antibody (Abcam ab15277, Cambridge, UK).

Mass Spectrometry-Based Identification of Dystrophin from Gel Bands

The Coomassie stained gel slice was excised and de-stained using 50% acetonitrile (ACN) in 50 mM NH₄HCO₃. Proteins were subjected to in-gel digestion. For reduction the gel piece was incubated in 45 mM DTT/50 mM NH4HCO3 for 30 min at 55° C. Alkylation of sulfhydryl (—SH) groups was done by incubation of the gel slice in 100 mM iodoacetamide/50 mM NH4HCO3 at RT in the dark for 30 min. Digestion was carried out using 70 ng LysC (FUJIFILM Wako Chemicals Europe, Neuss, Germany) for 4 h at 37° C. followed by a second digestion step using 70 ng porcine trypsin (Promega, Fitchburg, Wis., USA) overnight. Peptides were extracted using 70% ACN. Prior to mass spectrometry analysis the samples were dried using a SpeedVac vacuum concentrator. The tryptic peptides were separated on an Ultimate 3000 nano-LC system (Thermo Fisher Scientific, MA, USA) and identified on an online coupled Q Exactive HF-X mass spectrometer (Thermo Fisher Scientific). For separation, a 50 cm column was used (Column: PepMap RSLC C18, 75 μm×50 cm, 2 μm particles, Thermo Scientific) and a 160 min gradient from 5% solvent A (0.1% formic acid in water) to 25% solvent B (0.1% formic acid in acetonitrile) followed by a 10 min gradient from 25% to 40% solvent B. For MS measurement a top 15 data dependent CID method was used. MS data were searched using MASCOT V2.6.1 (Matrix Science, London, UK) against the porcine subset of the NCBI refseq database and filtered for an FDR <1%. Data were further validated using Scaffold V4 (Proteome Software, Portland, Oreg.).

### Selected Reaction Monitoring (SRM) Analysis

Sample preparation for selected reaction monitoring was carried out as for the mass spectrometry-based identification of dystrophin described in the chapters before, with the difference that 50 fmol of the synthetic heavy peptides (JPT, Berlin, Germany) were spiked in prior to digestion. SRM runs were performed on a nanoACQUITY UPLC system (Waters, Milford, Mass., USA) coupled to a triple-quadrupole linear ion trap mass spectrometer (QTRAP 5500, AB SCIEX, Framingham, Mass., USA). Tryptic peptides were transferred to a trap column (PepMap100 C18, 5 μm, 300 μm i.d.×5 mm, Thermo Scientific) at a flow rate of 10 μl/min and separated at 280 nl/min on a reversed-phase C18 nano-LC column (ReproSil-Pur 120 C18-AQ, 2.4 μm, 75 μm i.d.×15 cm, Dr. Maisch, Ammerbuch-Entringen, Germany). The following consecutive linear gradients were used: 1-5% B (0.1% formic acid in acetonitrile) in 1 min, 5-35% B in 45 min and 35-85% B in 5 min. For every peptide, three transitions were measured and chromatograms were evaluated using Analyst V 1.5.1. (AB SCIEX, Framingham, Mass., USA).

### Holistic Proteome Analysis of Skeletal Muscle Samples:

Protein concentration of the lysates was adjusted to a concentration of 2.3 μg/μl using 8 M Urea/0.4 M NH4HCO3. 250 μg of total protein was reduced using DTE at a final concentration of 5 mM for 30 min at 37° C. Cysteins were alkylated at room temperature for 30 min in the dark with iodoacetamide (final concentration 15 mM). Proteins were digested for 4 h at 37° C. using 2.5 μg LysC (FUJIFILM Wako Pure Chemicals, Osaka, Japan). The samples were diluted with water to 1 M urea and digested overnight with 5 μg porcine trypsin (Promega, Madison, Wis., USA) at 37° C. 1.5 μg of tryptic peptides were subjected to LC-MS/MS analysis as described above. For protein identification (FDR <1%) and label free quantification, the acquired spectra were analyzed using the MaxQuant software platform (V1.6.1) in combination with the porcine subset of the NCBI refseq database. Hierarchical clustering, principal component analysis and Student's t-test were calculated with Perseus (V 1.5.3.2) part of the MaxQuant proteomics pipeline⁴⁵. The mass spectrometry proteomics data have been deposited to the ProteomeXchange Consortium via the PRIDE⁴⁶ partner repository with the dataset identifier PXD014893.

hiPSC Reprogramming and Culture

The DMDΔ52 human iPSC line was reprogrammed with the CytoTune-iPS 2.0 Sendai Reprogramming kit (Invitrogen A16517), as previously described⁴³, using the peripheral blood mononuclear cells (PBMCs) of a male Duchenne muscular dystrophy patient carrying a deletion of DMD exon 52 leading to a premature stop codon. The healthy hiPSC line was reprogrammed from the PBMCs of a young, male volunteer following the same protocol. All recruitment and consenting procedures were done under institutional review board-approved protocols of both the Klinikum rechts der Isar, Technical University of Munich, and the Klinikum of the Ludwig-Maximilian University, Munich. Written informed consent was obtained from the affected patient and healthy volunteer.

Pluripotency was assessed after reprogramming via alkaline phosphatase staining (Roche 11681451001), immunofluorescence analysis of the pluripotency markers Nanog and TRA-1-81 (all antibodies listed in Table 3) and qPCR analysis of the pluripotency markers OCT4, SOX2, NANOG, REX1 and TDGF-1, as previously described²². Germ-layer differentiation potential was tested via spontaneous embryoid body differentiation in DMEM/F12 medium containing 20% FBS, 50 μg/mL L-ascorbic acid (Sigma-Aldrich A5960), 1% L-glutamine, 1% non-essential amino acids and 0.5% Penicillin-Streptomycin for 21 days followed by qPCR analysis of markers of endoderm (SOX7, AFP), mesoderm (CD31, DES, ACTA2, SCL, CDH5) and ectoderm (KRT14, NCAM1, TH, GABRR2) using GAPDH as an endogenous control. Loss of Sendai virus was confirmed after 13 passages via immunofluorescence analysis and RT-PCR of the Sendai vector and viral transgenes OCT4, SOX2, KLF4 and c-MYC using GAPDH as an endogenous control.

Karyotyping was performed by the Institute of Human Genetics of the Klinikum rechts der Isar, Technical University of Munich. hiPSCs were maintained in mTeSR1 medium (Stemcell Technologies 85850) on Matrigel-coated plates (Corning 354277).

TABLE 3
Antibodies.

| Target | Host | Reference | Concentration |
|---|---|---|---|
| Nanog | Rabbit | Abcam ab21624 | 1:500 (IF) |
| TRA-1-81 | Mouse | BD Pharmingen 560174 | 1:20 (IF) |
| Sendai virus | Rabbit | MBL PD029 | 1:1000 (IF) |
| Dystrophin | Rabbit | Abcam ab15277 | 1:100 (IF), 1:12.5 (Wes) |
| α-actin | Mouse | Abcam ab3280 | 1:25 (Wes) |
| α-actinin | Mouse | Sigma-Aldrich A7811 | 1:250 (IF) |
| Myosin heavy chain β | Mouse | Chemicon MAB1548 | 1:50 (IF) |
| Anti-Mouse IgG A488 | Goat | Invitrogen A32723 | 1:500 (IF) |
| Anti-Mouse IgG A647 | Goat | Invitrogen A32728 | 1:500 (IF) |
| Anti-Rabbit IgG A488 | Goat | Invitrogen A32731 | 1:500 (IF) |

RNP-Mediated CRISPR/Cas9 Deletion of DMD Exon 51 in DMDΔ52 hiPSCs (DMDΔ51-52 Line)

For CRISPR/Cas9-mediated deletion of DMD exon 51 in DMDΔ52 hiPSCs, the Alt-R CRISPR-Cas9 system (IDT) was used according to the manufacturer's instructions. Briefly, crRNA oligonucleotides targeting the human DMD exon 51 locus (TAATTTGAAGCTGGACCCTA (SEQ ID NO: 33) and GTCTAGGAGAGTAAAGTGAT (SEQ ID NO: 34)) were purchased from IDT and duplexed with fluorescently labeled tracrRNA (IDT 1075927). The obtained gRNAs (Table 2) were then each used to generate equimolar ribonucleoprotein (RNP) complexes with the S. pyogenes Cas9 protein (IDT 1074181) in Opti-MEM medium (Gibco 31985062). The RNP complexes were then reverse transfected into DMDΔ52 hiPSCs dissociated with TrypLE Express (Gibco 12604013) using Lipofectamine Stem Transfection reagent (Invitrogen STEM00003). A final RNP concentration of 10 nM was applied for 4×10⁵ cells per well of a Matrigel-coated 96 well plate. Transfected cells were dissociated into single cells 24 hours after transfection with a 10-minute Accutase treatment (Gibco A1110501) and 1000 cells were seeded into a Matrigel-coated 10 cm plate in mTeSR1 containing 10 μM Y27632 (Calbiochem 688000). mTeSR1 was replaced every day until colonies were large enough to cut in half for clone screening and passaging. Deletion of exon 51 was verified by PCR and Sanger sequencing by Eurofins Genomics. The generated DMDΔ51-52 line was confirmed to have a normal karyotype by the Institute of Human Genetics of the Klinikum rechts der Isar, Technical University of Munich.

hiPSC Muscle Differentiation

Skeletal muscle differentiation of hiPSCs was induced using a commercially available kit (Amsbio SKM-KITM). Briefly, hiPSCs were dissociated with Accutase on day 0 and seeded into plates coated with 5 μg/cm² type I collagen (Cell applications 122-20) at a density of 5000 cells/cm² in Skeletal Muscle Induction Medium (Amsbio SKM01). Myogenic precursors were obtained within 6-8 days, at which point the cells were dissociated with TrypLE Express (Gibco 12604013) and replated into type I collagen coated plates at a density of 5000 cells/cm² in Skeletal Myoblast Medium (Amsbio SKM02). The cells reached the myoblast stage within 6-8 days, after which the medium was replaced with Myotube Medium (Amsbio SKM03) to induce the formation of skeletal muscle myotubes. After 5 days, the Myotube Medium was replaced with Skeletal Muscle Cell Differentiation Medium containing 2% horse serum (Promocell C-23061; C-39366). Myotubes were maintained in culture a total of 7 or 14 days from the switch to Myotube Medium.

Differentiation into cardiomyocytes was induced by modulation of Wnt/β-catenin signaling, following a protocol described by Lian and colleagues, with some modifications⁴⁴. Briefly, hiPSCs were seeded onto 12-well plates coated with 2 μg/cm² fibronectin (Sigma-Aldrich F1141) at a density of 2×10⁵ cells/well. Upon reaching 90%, confluence after 3-4 days, cardiac differentiation was induced on day 0 by changing to RPM11640 (Gibco 21875091) with B27 minus insulin (Gibco A1895601) (defined as basal cardiac differentiation medium) supplemented with 6 μM CHIR99021 (Axon Medchem 1386). On day 2, medium was replaced with basal cardiac differentiation medium supplemented with 5 μM IWR1 (Tocris 3132). After maintaining the cells in basal cardiac differentiation medium another 14 days, beating areas were mechanically transferred to fibronectin-coated plates and cultured in DMEM/F-12 (Gibco 11320033) with 2% FBS, 1% non-essential amino acids (Gibco 11140050), 1% Penicillin-Streptomycin-Glutamine (Gibco 10378016) and 0.1 mM β-mercaptoethanol.

### AAV-Mediated CRISPR/Cas9 Deletion of DMD Exon 51 in Patient-Derived Muscle Cells

Skeletal myoblasts and cardiomyocytes derived from DMDΔ52 patient hiPSCs were transduced with a dual AAV system (AAV2/6-Cas9/gE51) carrying the sequences for the split-intein Cas9 protein used in the pig model and two gRNAs targeting the human DMD exon 51 locus (Table 2 and FIG. 11). Both AAVs were added to the cells at a concentration of 10⁶ particles per cell and removed after 20 hours. Transduced myoblasts were maintained in Skeletal Myoblast Medium for 6 days before inducing skeletal muscle myotube formation as described above.

### Immunofluorescence Analysis

Immunofluorescence staining was performed as previously described⁴⁵ using the primary antibodies listed in Table 3 herein. Sample imaging was performed with an inverted or confocal laser scanning microscope (DM16000B and TCS SP8, Leica Microsystems, Wetzlar, Germany).

### PCR, RT-PCR and Quantitative Real-Time PCR

PCR analysis to study genomic editing in DMD pigs was performed on genomic DNA extract from various tissues using the Wizard® Genomic DNA Purification Kit (Promega) and Q5 polymerase (NEB). RT-PCR was performed on Trizol (Invitrogen, #15596-026) or RNeasy Mini Kit (Quiagen) extracted RNA samples from snap frozen tissue. Reverse transcription was performed using random hexamers and SuperScript®-VILO (Invitrogen, #11904-018) according to the manufacturers instructions. Quantitative DMD analysis was performed using genomic DNA from different regions and the ABI PRISM 7900 Sequence Detection System (Applied Biosystems) and TaqMan® reaction mixes for detecting unedited (APKA34W, Applied Biosystems) versus exon 51 deleted DMD (APMFXPU, Applied Biosystems). All samples were measured in triplicates in a 20 μl reaction contained 10 μl of TaqMan® Universal PCR Master Mix (Applied Biosystems), 60 ng of HindIII fragmented gDNA template, 300 nmol/L of each primer and 200 nmol/L of the specific FAM-labeled probe. The fluorescent signal intensities were recorded and analyzed during PCR amplification using the Sequence Detection Software (SDS, Applied Biosystems) software. Following, the ratio of unedited versus edited DMD was determined (2^{-[ΔCT_del-ΔCT_WT}]). Data was analyzed using Sigma Plot 12.0 (Systat Software, Inc, Chicago, USA) and GraphPad Prism 6.0 (GraphPad Software, La Jolla, USA). Differences between two independent groups were analyzed using the t-test or the Mann-Whitney U test. For comparing more than two groups a two-way ANOVA was performed. For post hoc tests the Holm-Sidak Test was applied. The chosen level of significance was p≤0.05; results with p-values between 0.05 and 0.1 were described as tendencies not reaching statistical significance.

For analyses in cells, genomic DNA was isolated from cells with the Gentra Puregene kit (Qiagen 158722) and PCR was performed with the Q5 High-Fidelity DNA Polymerase (NEB M0491S). For RT-PCR and Real-time qPCR, total RNA was extracted with the Absolutely Microprep kit (Agilent 400805) and cDNA was produced with the High Capacity cDNA RT kit (Applied Biosystems 4368814). PCR was performed with the FIREPol DNA Polymerase (Solis Biodyne 01-01-00500). Real-time qPCR was performed with a 7500 Fast Real-time PCR system (Applied Biosystems, Germany) using the Power SYBR Green PCR Master Mix (Applied Biosystems 4367659) and primers. Data was analyzed using the 2^{-ΔΔCt} method with normalization to GAPDH expression.

### Capillary Western Immunoassay

Cells were lysed in RIPA buffer (Sigma-Aldrich R0278) containing proteinase inhibitor (Roche 11836170001) and total protein concentration was determined via Pierce BCA assay (Thermo Fisher 23225). Dystrophin levels were analyzed with the size-based Wes system (ProteinSimple) using an antibody targeting the C-terminus of human dystrophin and an antibody targeting a-actin as a loading control. Samples were loaded at a protein concentration of 0.05 mg/mL or 0.1 mg/mL for hiPSC-derived skeletal myotubes and cardiomyocytes, respectively.

### Ex Vivo Myocardial Cultivation

For ex vivo heart slice cultivation, porcine myocardial tissue was obtained from left mid-ventricular transmural sections and immediately placed in a 30 mM 2,3-butadione-2-monoxime solution (BDM, Sigma-Aldrich B0753) at 4° C. The sections were embedded in 5% agarose and further processed to 300 μm thick tissue slices by vibratome cutting (VT1200S, Leica Biosystems, Germany). Slices were anchored in biomimetic culture chambers via small plastic triangles attached to the slices with tissue adhesive (Histoacryl, B. Braun 69390) according to the fiber direction and immediately subjected to physiological preload of 1 mN and stimulation at 0.5 Hz (50 mA pulse current, 1 ms pulse duration). Before calcium imaging, the slices were maintained for 24 hours in M199 medium (Sigma-Aldrich M4530) supplemented with 1% Penicillin-Streptomycin, 0.5% insulin/transferrin/selene and 50 μM β-mercaptoethanoI on a rocker plate (60 rpm, 15° C. tilt angle) placed in an incubator set at 37° C., 5% CO₂, 20% O₂ and 80% humidity. A continuous readout of contraction force was obtained via the biomimetic chamber²¹.

### Calcium Imaging

Myocardial tissue slices were incubated in culture medium containing 3 μM Fluo-4-AM (Thermo Fisher F14201), 0.75% Kolliphor EL (Sigma-Aldrich C5135) and 30 mM 2,3-butadione-2-monoxime (BDM, Sigma-Aldrich B0753) for 60 min at 37° C., then washed and incubated in Tyrode's solution supplemented with Ca2+(135 mM NaCl, 5.4 mM KCl, 1 mM MgCl2, 10 mM glucose, 1.8 mM CaCl₂), and 10 mM HEPES; pH 7.35) containing 30 mM BDM for another 30 min at 37° C. Spontaneous calcium signals from the tissue slices were subsequently imaged using an upright epifluorescence microscope (Zeiss Axio Examiner) equipped with a 40× objective, a GFP filter set, and a Rolera em-c² EMCCD camera.

For calcium imaging in hiPSC-derived cardiomyocytes, two months-old cells were dissociated to single cells using a papain-based protocol described previously⁴⁶, Cells were then seeded onto 3.5 cm glass bottom cell culture microdishes (MatTek Corporation P35G-1.5-14-C) coated with 2 μg/cm² fibronectin (Sigma-Aldrich F1141) at a density of 5×10³ cells/cm². Ten days after seeding, loading with 2 μM Fluo-4-AM (Thermo Fisher F14201) for 30 min at 37° C., de-esterification of the dye for 30 min at 37° C. and imaging were all performed in Tyrode's solution supplemented with Ca²⁺ (135 mM NaCl, 5.4 mM KCl, 1 mM MgCl2, 10 mM glucose, 1.8 mM CaCl₂), and 10 mM HEPES; pH 7.35). The glass bottom microdishes were placed on the stage of an inverted epifluorescence microscope (DM16000B, Leica Microsystems, Wetzlar, Germany), equipped with GFP filter sets, a HCX PL APO 63X/1.4-0.6 oil immersion objective (Leica Microsystems) and a Zyla V sCMOS camera (Andor Technology, Belfast, UK). Field stimulation electrodes (RC-37FS, Warner Instruments, Hamden, Conn., USA) were connected to a stimulus generator (HSE Stimulator P, Hugo Sachs Elektronik, March-Hugstetten, Germany) providing depolarizing pulses (50 V, 5 ms duration) at 1 Hz as indicated.

Imaging settings (illumination intensity, camera gain, binning) were adjusted to achieve an optimal signal-to-noise ratio while avoiding pixel saturation. Imaging rates were 14 Hz in the tissue slices and 100 Hz in the iPSC-derived cardiomyocytes. ImageJ (National Institutes of Health, Bethesda, Md.) was used to quantify fluorescence over single cells and over background regions. Subsequent analysis was performed in RStudio (RStudio Team (2015). RStudio: Integrated Development for R. RStudio, Inc., Boston, Mass.) using custom-written scripts. After subtraction of background fluorescence, the time course of Fluo-4 fluorescence was expressed either in arbitrary units or normalized to the initial value (F/F₀). After manual selection of the starting points and the peaks of the calcium transients, the transient duration at 90%, decay (TD₉₀), the rise time and the monoexponential decay time constant A were automatically determined by the script.

### Off-Target Analysis

Potential off-targets with up to four mismatches have been identified using the CRISPOR web tool³⁶ and ranked according to their CFD and MIT score. The three highest off-targets for both the intron 50 and intron 51 gRNA has been amplified by PCR and sanger sequenced using genomic DNA from a control pig, intramuscular treated animals, different tissues from a systemically injected pig, as well as hDMDΔ52 hiPSCs, hDMDΔ51-52 hiPSCs, and hDMDΔ52 hiPSC-derived myoblasts after infection with AAV2/6-Cas9/gE51.

Statistical methods

The results are given as mean±SEM, if not indicated otherwise. Statistical analysis of results between >2 experimental groups was performed with one way analysis of variance ANOVA. Whenever a significant effect was obtained with ANOVA, we performed multiple comparison tests between the groups using the Student Newman Keul's procedure (parametric) or Tukey's test (non-parametric). Two experimental groups were compared by Student's T-Test. For non-parametric testing, the Mann Whitney U test was performed. Kaplan-Meier analysis was used in FIG. 3a. All procedures were performed with an SPSS statistical program (Version 25). Differences between groups were considered significant for p<0.05.

### Example 1

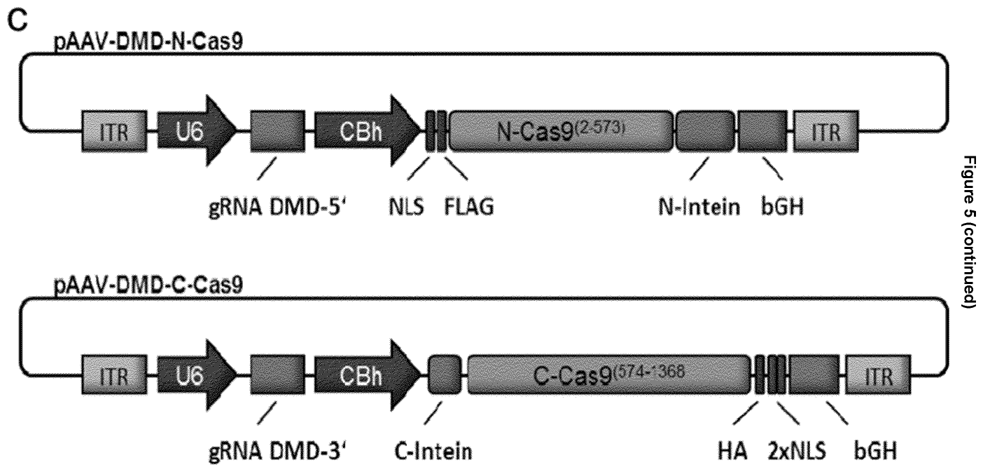

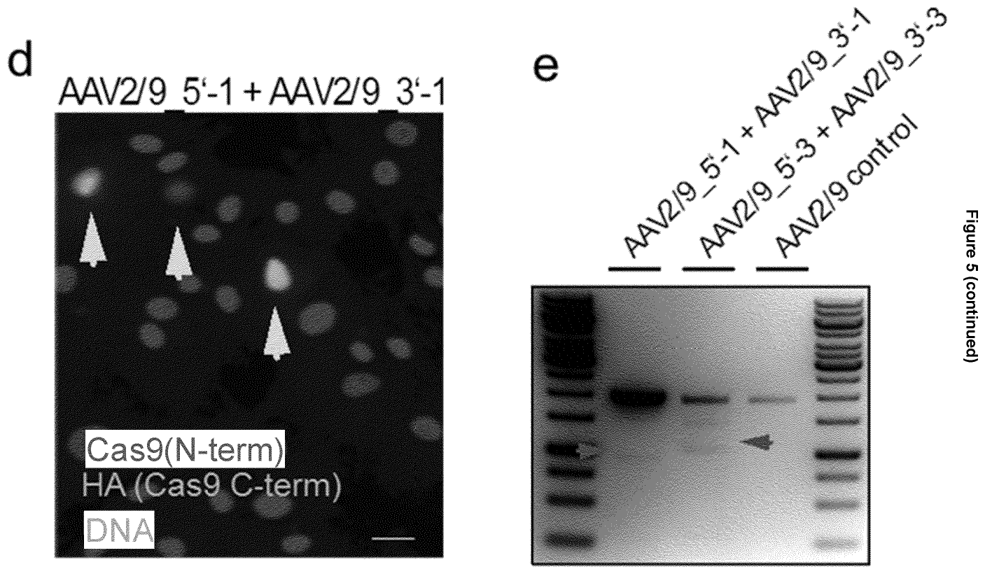

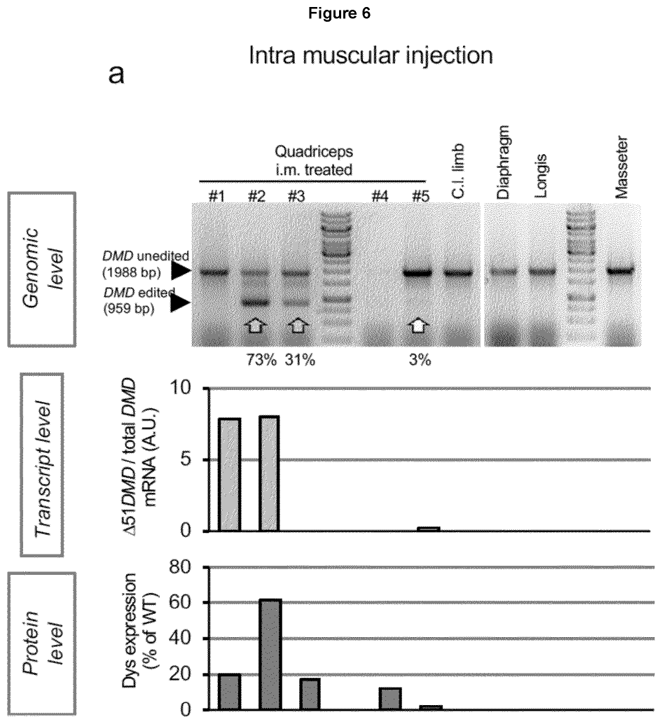

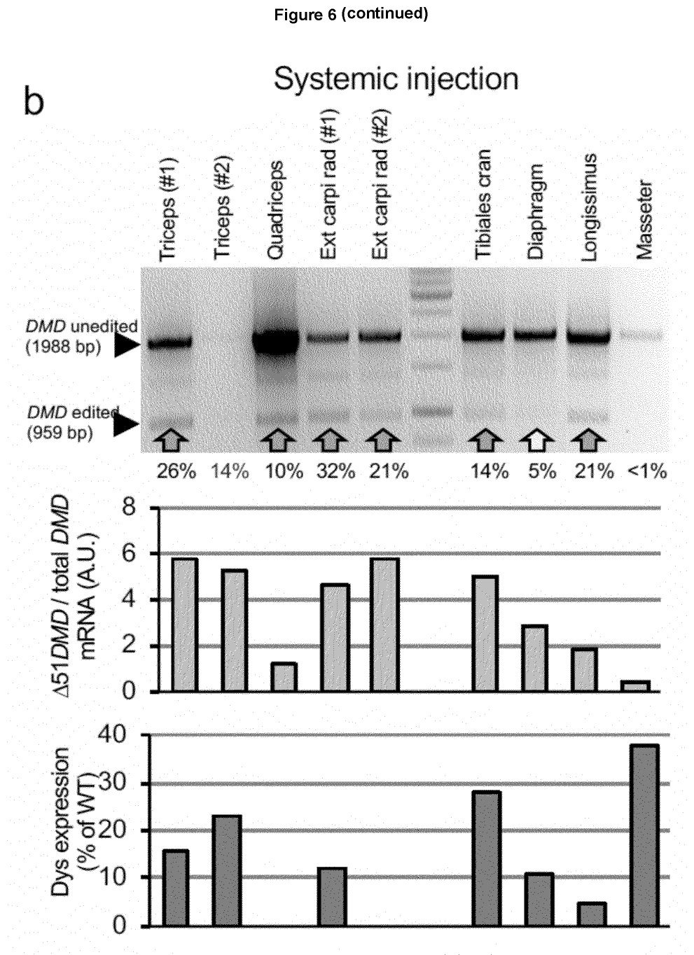

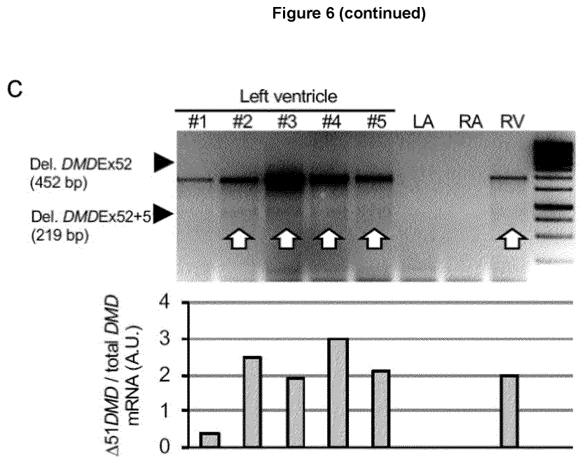

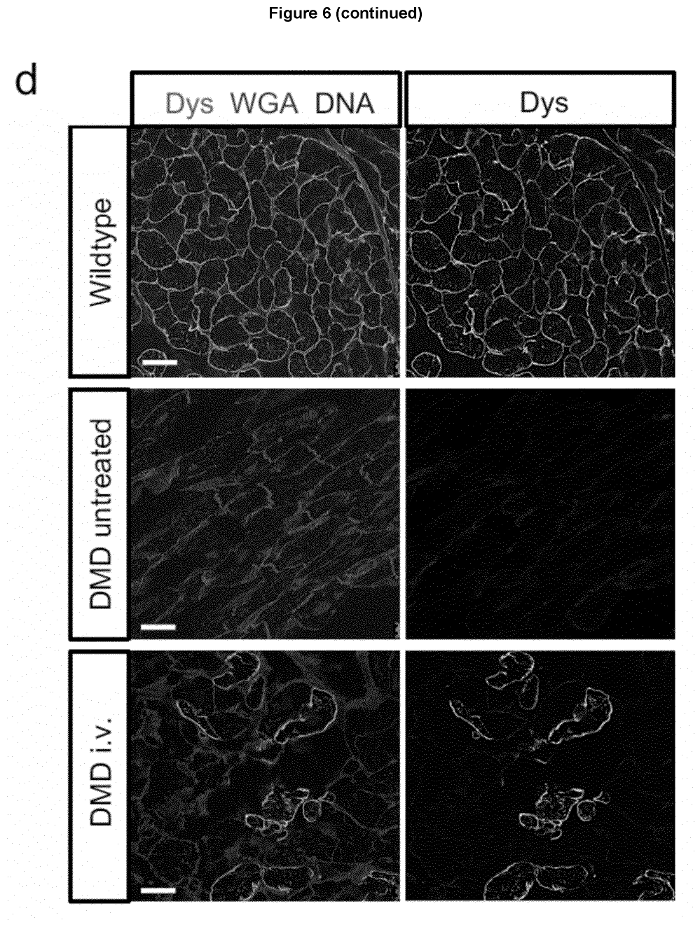

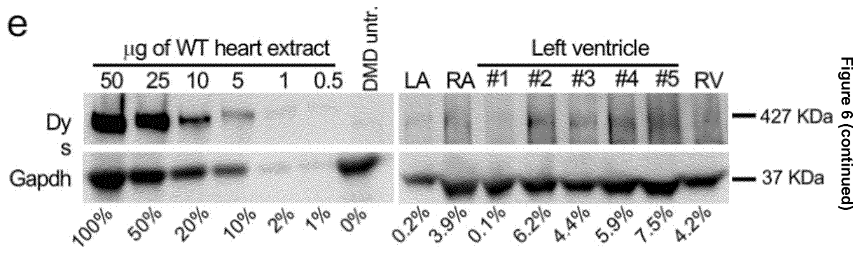

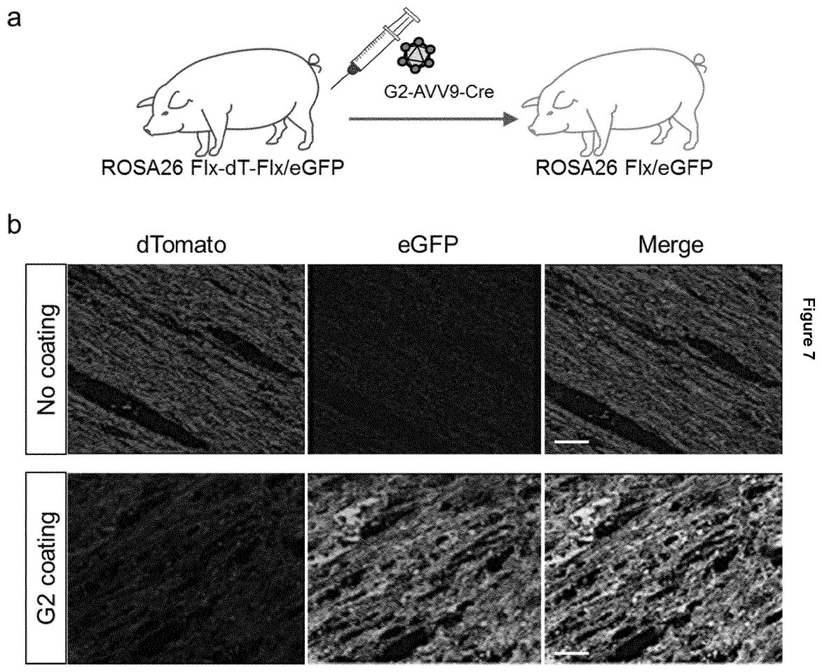

The inventors have generated a DMD pig model by replacing DMD exon 52 with a neomycin-resistance cassette¹, resulting in a complete loss of dystrophin expression (FIG. 1a, Methods). First, the inventors assessed whether local application of intein-split Cas9-vectors and selected gRNAs targeting exon 51 (FIG. 5) is efficiently excising this exon and enabling expression of a DMDΔ51-52 in vivo (FIG. 1). Ten to fourteen-day-old piglets were subjected to unilateral fore- and hindlimb intramuscular (i.m.) injection of a pair of intein-split Streptococcus pyogenes (Sp)-Cas9² and gRNA-encoding virus particles (2×10¹³ vp/kg each). After six weeks, histological analysis revealed restitution of membrane-localized dystrophin in the treated areas and successful elimination of exon 51 was confirmed at genomic, transcript, and protein levels (FIG. 1b-d and FIG. 6). Immunoblotting demonstrated expression of DMDΔ51-52 in the treated muscles, which was barely detectable in untreated contralateral tissue (FIG. 1d). Importantly, the global proteome profile of injected muscles resembled more closely that of healthy rather than DMD animals (FIG. 1e,f). Of note, several collagens and fibronectins related to fibrosis were significantly reduced after treatment (FIG. 8a).

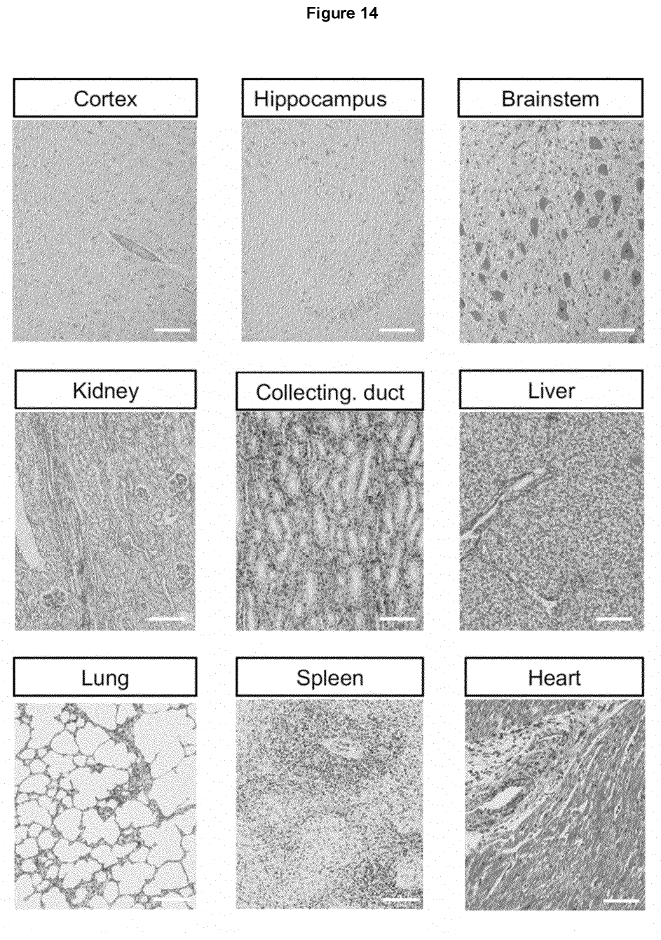

Since the diaphragm and the heart—two muscles contributing substantially to the mortality of Duchenne patients^{16,17}—have not been transduced by i.m. limb injection (FIG. 1b), the inventors sought to treat these organs by systemic AAV9-Cas9-gE51 application. For this purpose, the inventors enhanced the transduction efficacy of the AAV by coating the virus with PAMAM-G2 nanoparticles¹⁸ without altering the myotropism¹⁹ of the vector (FIG. 7) and without enhancing toxicity (FIG. 14). A dose-finding study indicated that intravenous (i.v.) application of 1-2×10¹³ coated-vp/kg (low dose) sporadically transduced skeletal muscle specimen (FIG. 1b); however, i.v. application of 2×10¹⁴ vp/kg of each of the G2-AAV9-Cas9-vectors restored dystrophin protein expression in all muscles investigated, including the diaphragm and heart, as assessed by immunohistochemistry, immunoblot, and mass spectrometry (FIG. 1b,d, FIG. 8a,b). In muscles, the inventors measured up to 26% DMD gene editing (FIG. 6b) and up to 56.6% of DMD transcripts lacked exon 51, while no genome editing of DMD was found in remote organs such as liver, spleen and kidney (FIG. 1c, FIG. 8c). No off-target effects were detected for the 5 most likely predicted targets by deep sequencing.

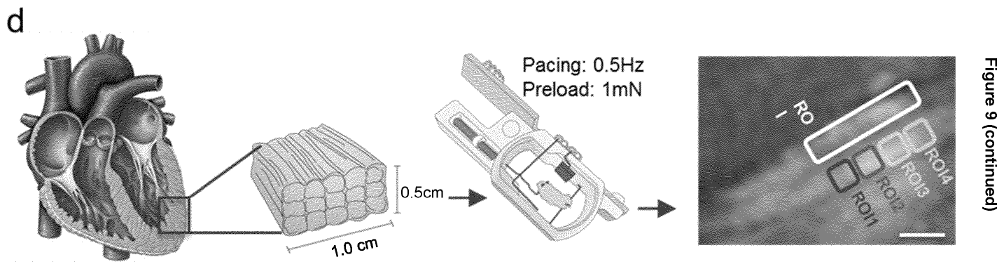

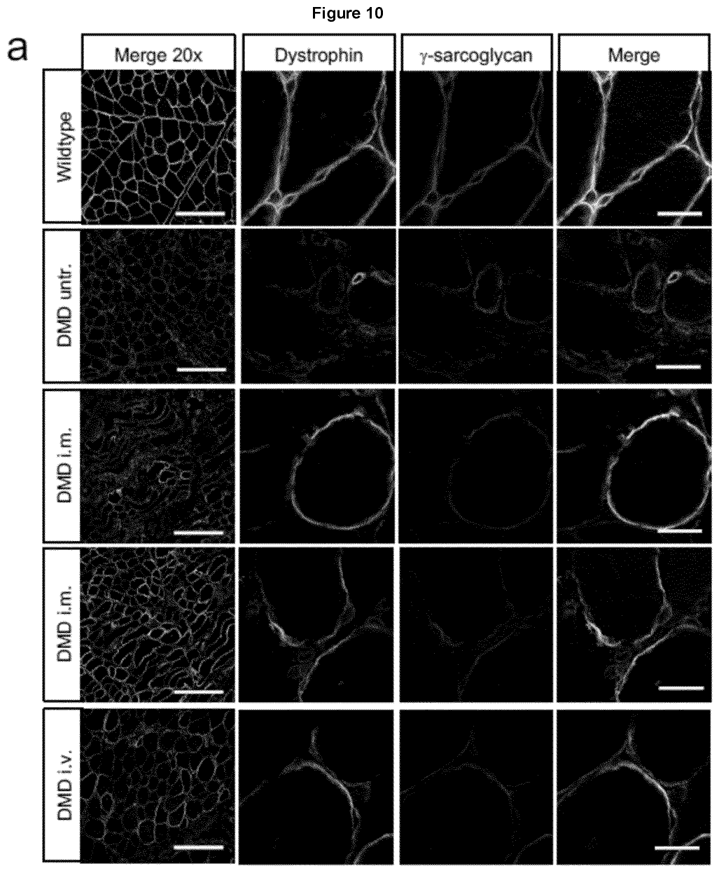

Lack of dystrophin in DMD muscles induces the collapse of the dystrophin-associated glycoprotein complex (DGC). Local i.m. or high-dose i.v. treatment resulted in increased level of membrane-bound amount of β-dystroglycan and γ-sarcoglycan and restoration of the DGC (FIG. 10). Further structural evaluation of skeletal muscles revealed that expression of DMDΔ51-52, which co-localized with the membrane marker wheat germline agglutinin (WGA), significantly reduced occurrence of rounded myofibers with centralized nuclei (FIG. 2a-c). Moreover, the inventors measured a significant increase of capillary density and reduction of both mononuclear cell infiltration and interstitial fibrosis in treated skeletal muscles compared to untreated controls (FIG. 2d-g, FIG. 8), indicating improved blood perfusion and tissue integrity. Indeed, muscular function was ameliorated by G2-AAV9-Cas9-gE51 application, as demonstrated by augmented muscle twitch amplitude and tetanic contraction force (FIG. 2h,i).

It is worth mentioning that in the DMDΔ52 animal cohort 61.6% (45/73) of the affected males died within the first week after birth and none survived longer than 105 days. The maximal survival of animals, which received i.v. G2-AAV9-Cas9-gE51-treatment at 4 weeks after birth, increased significantly to 136 days (FIG. 3a). In order to better understand the high mortality of untreated DMDΔ52 pigs—that were documented to succumb to sudden cardiac death by videotaping—the inventors investigated the global function and arrhythmogenicity of DMD hearts. Left ventricular (LV) angiography showed a reduction of ejection fraction in DMDΔ52 animals compared to wildtype controls, which was not significantly attenuated by systemic G2-AAV9-Cas9-gE51-treatment (p=0.08). Contraction (dP/dtₘₐₓ) and relaxation velocity (dP/dtₘᵢₙ) were not significantly different (FIG. 3b). However, no overt signs of heart failure (e.g. pleural effusion, pulmonary edema) were observed in the instance of death in DMDΔ52 animals (data not shown), suggesting malignant arrhythmias as primary cause of death. Indeed, detailed electrophysiological mapping analysis (FIG. 9a-c) demonstrated a decrease of voltage amplitude in ventricles of DMDΔ52 pigs compared to wildtype hearts (FIG. 3c), indicating cardiac fibrosis²⁰. Histological assessment confirmed increased formation of fibrotic areas as a potential substrate of low amplitude regions (FIG. 3d, 9c). Systemic injection of high dose G2-AAV9-Cas9-gE51 significantly ameliorated the excitation disturbance and the fibrotic degeneration (FIG. 3c,d and FIG. 9b,c).

To better evaluate the intrinsic arrhythmogenic vulnerability of DMD cardiomyocytes, we performed ex vivo intracellular Ca²⁺ analysis of single cardiomyocytes within 300 μm-thick heart slices maintained in biomimetic chambers (Methods, FIG. 9d)²¹. Compared to wildtype hearts, cells from untreated DMDΔ52 cardiac tissues displayed abnormal Ca²⁺ transients (FIG. 3e,f) and unsynchronized intracellular Ca²⁺ waves (FIG. 3g). These pathological features were almost completely abolished by systemic G2-AAV9-Cas9-gE51 application (FIG. 3e-g), supporting the notion that an inherent arrhythmogenic susceptibility of DMDΔ52-cardiomyocytes can be ameliorated by genomic snipping of exon 51.

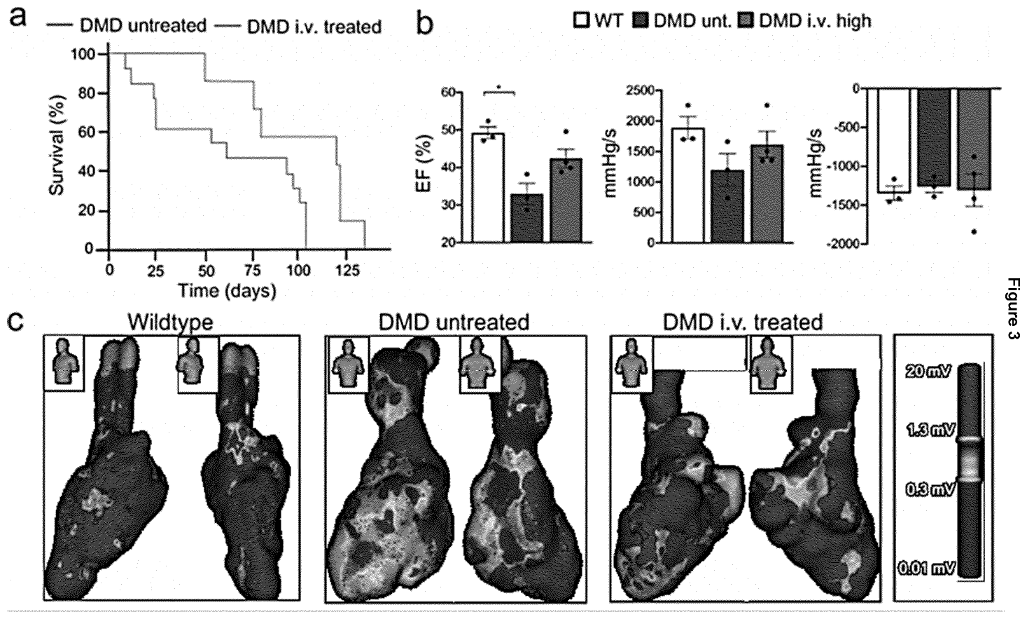

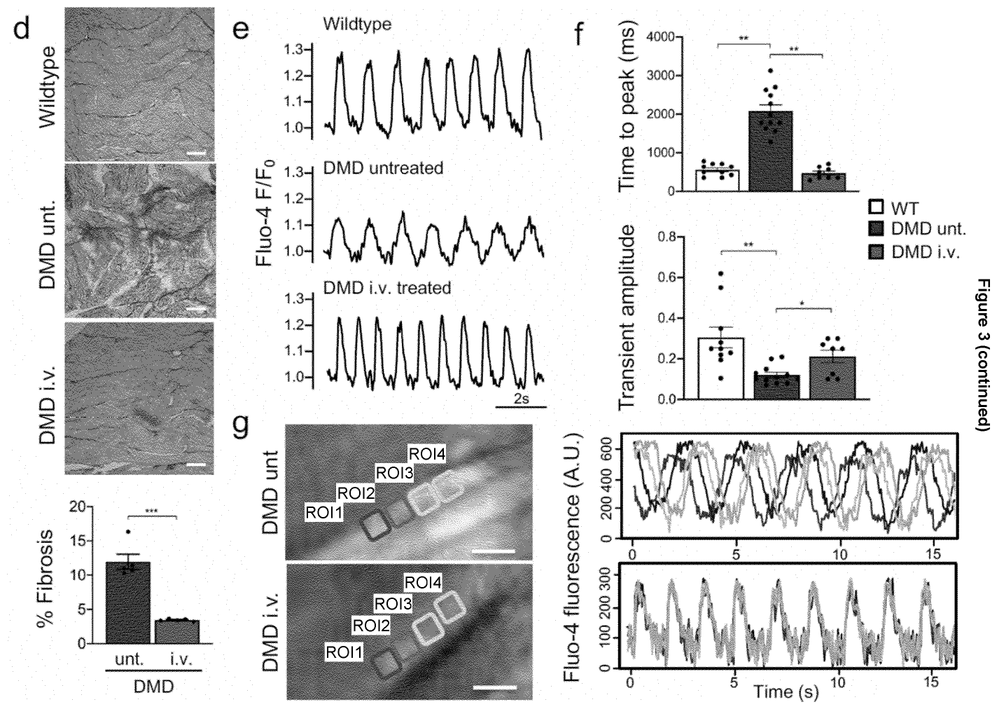

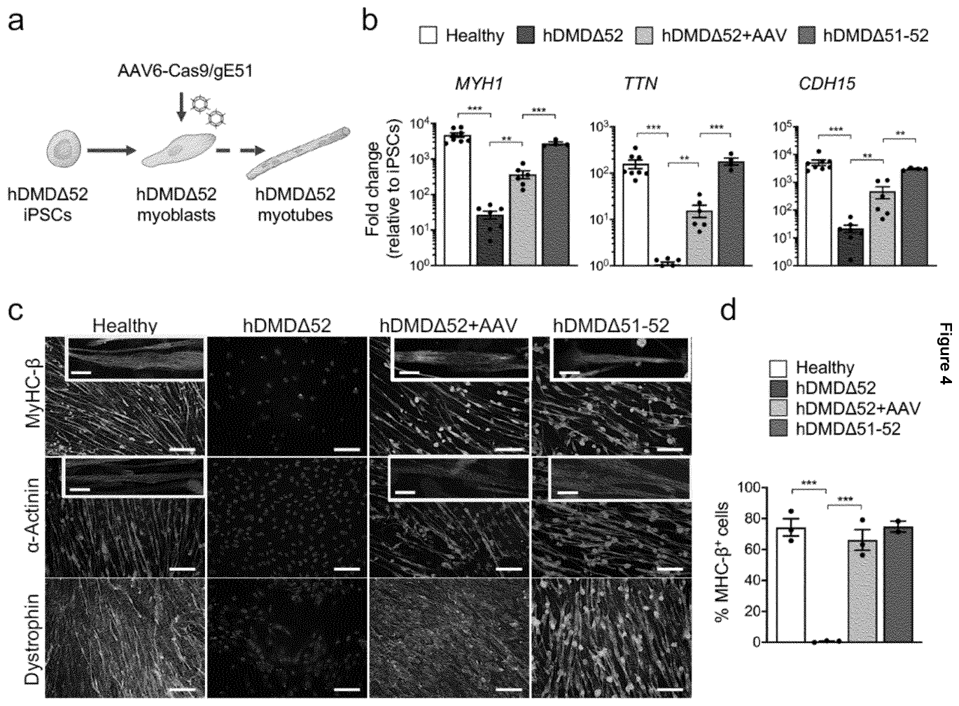

The inventors finally investigated the muscle-specific targeting efficacy of the intein-split Cas9 AAV-mediated approach in human cells (FIGS. 4 and 10). The inventors generated iPSCs²² from a DMD patient carrying likewise a deletion of DMD exon 52 (hDMDΔ52), leading to appearance of a premature stop codon in exon 53 (FIG. 11a-g). As controls, we used isogenic hDMDΔ51-52 iPSCs that were obtained by CRISPR/Cas9-mediated excision of exon 51 in the undifferentiated hDMDΔ52 iPSCs (FIG. 11h-i) and iPSCs from a healthy, young male (FIG. 12).

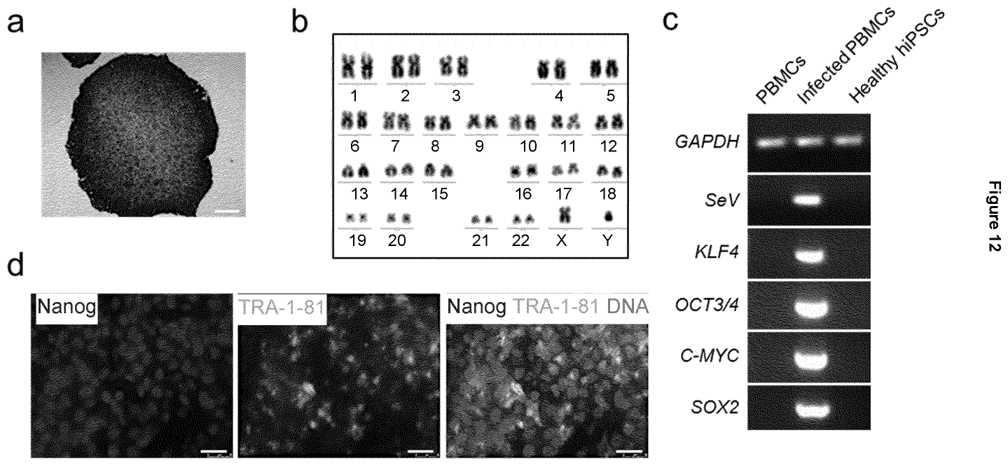

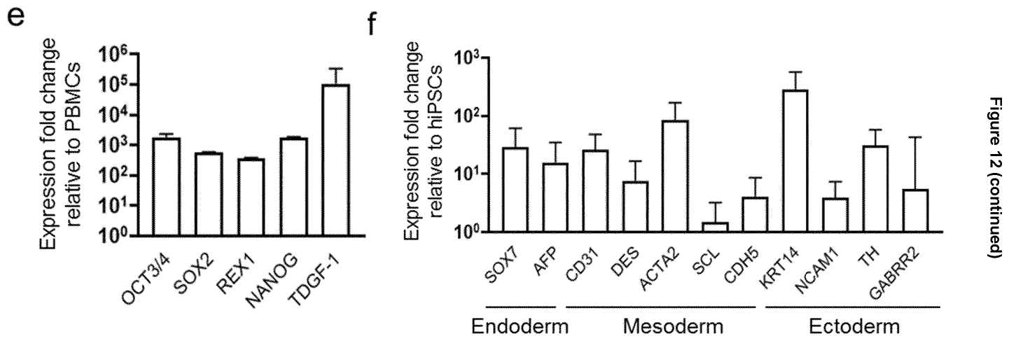

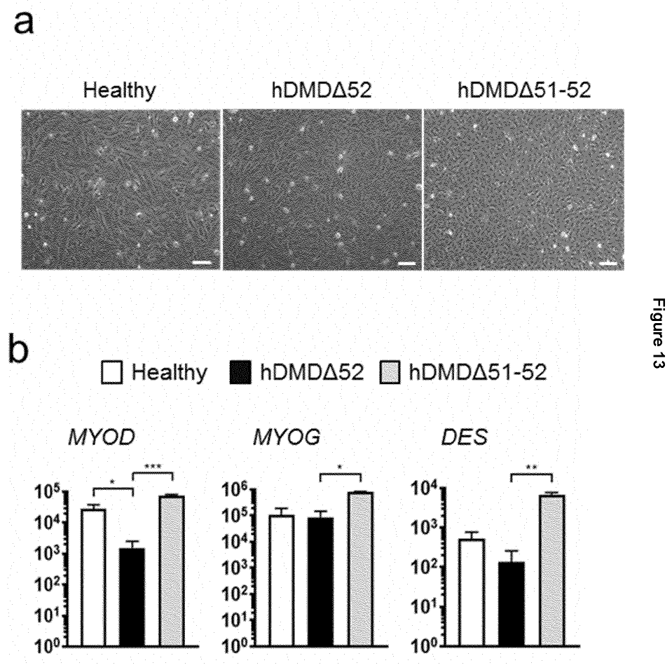

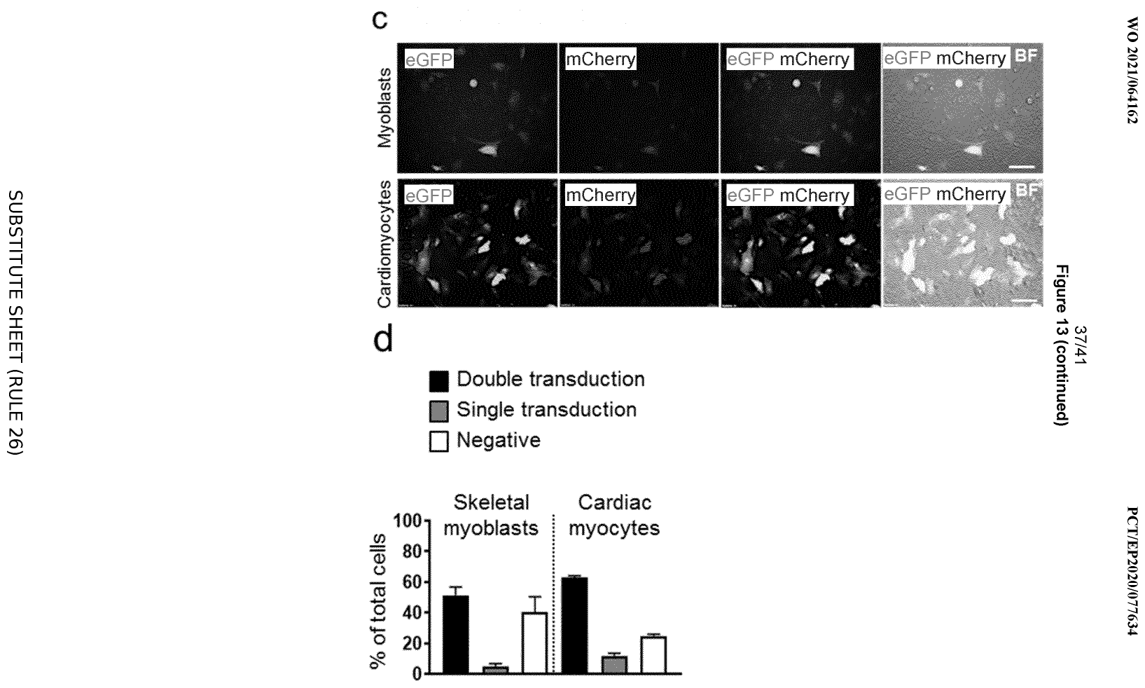

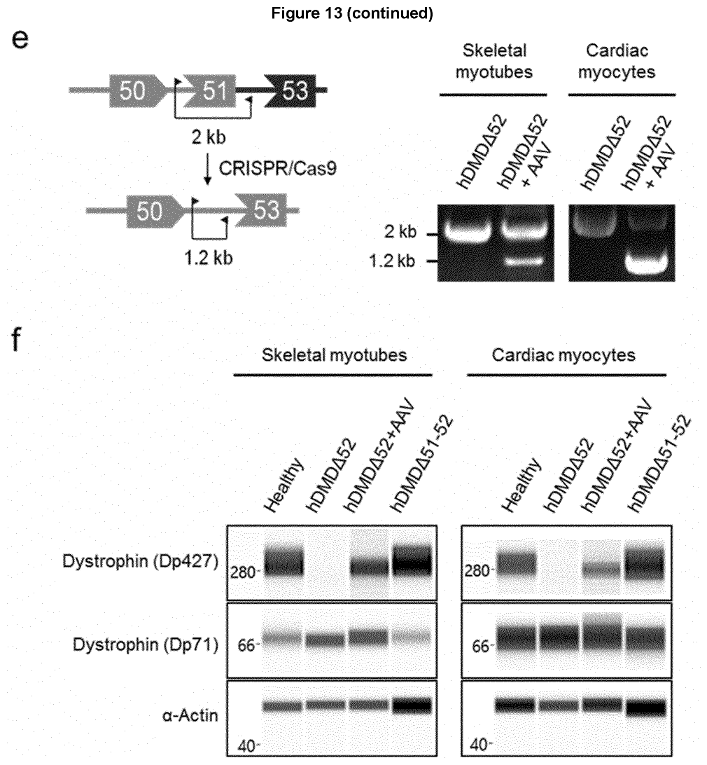

When forced to differentiate into skeletal muscle by a transgene-free approach, hDMDΔ52 cells expressed significantly lower levels of skeletal muscle genes (FIG. 4b) compared to controls and failed to generate multinucleated and spontaneously contractile myotubes (FIG. 4c,d). Infection of hDMDΔ52 iPSC-derived myoblasts—which represent a skeletal myogenic precursor that is formed also in presence of the DMDΔ52 mutation (FIG. 13a,b)—with a pair of AAV6 vectors expressing intein-split Cas9 and gRNAs targeting human DMD exon 51 (10⁶ vp/cell) restored expression of a re-framed DMDΔ51-52 protein and rescued myotube formation (FIG. 4a-d, FIG. 130. Co-infection yield was around 90%, as assessed using viruses carrying the CRISPR gene editing components together with a pair of fluorescence reporters (FIG. 13c,d). AAV6-Cas9-mediated exon 51 excision and expression of re-framed DMDΔ51-52 was also efficient in hDMDΔ52 iPSC-derived cardiomyocytes (FIG. 13e,f) and abolished Ca²⁺ handling defects and arrhythmogenic susceptibility in infected cells (FIG. 4e-h), corroborating the in vivo results. Of note, the inventors detected some dystrophin expression in hDMDΔ52 cells (FIG. 4c and FIG. 130, which corresponded to the ubiquitous, short isoform Dp71, whose promoter/first exon is located in intron 62^{23,24} and is thus not affected by the DMD exon 52 deletion. No off-target effects were detected by whole-genome sequencing in isogenic hDMDΔ51-52 iPSCs.

In summary, the inventors found that a large animal model of Duchenne muscular dystrophy, displaying disease hallmarks such as muscle weakness, cardiomyopathy and premature death, can be treated by somatic genome editing of the mutated DMDΔ52 gene via AAV9-Cas9-gRNA. Intramuscular therapy provided a robust expression of the internally truncated, but partially functional DMDΔ51-52 in the injected skeletal muscles, with minimal editing of other muscles (such as contralateral muscles, diaphragm and heart) or remote non-muscle organs (liver, lung, and kidney). Pharmacologic testing revealed that systemic application of up to 2×10¹⁴ vp/kg for each of the two AAV9 vectors produced efficient and broad muscle transduction, including diaphragm and heart. This effect was improved by dendrimer viral coating with G2-PAMAMs and allowed using lower virus amounts that do not exceed a recently reported level of toxicity in non-human primates and piglets²⁵. No intracellular off-target effects were detected at this systemic dose in highly transduced peripheral muscle tissue, as previously demonstrated with the same dose (2×10¹⁴ vp/kg) in a dog model of DMD¹⁵. Although transduction of satellite cells has been demonstrated before in mdx mice¹⁴ expressing a reporter gene under the control of the Pax7 promoter, the inventors got no evidence for such events in pigs with their viral system.

Using iPSCs, it was possible to model the effects of the human DMDΔ52 mutation and evaluate the efficacy of AAV-Cas9-mediated somatic excision of exon 51 in human muscle cells, which could theoretically reframe about 14% of total DMD mutations^{27,28}. For the first time, we demonstrate that restoration of dystrophin by genome editing can be achieved at the level of myoblasts as well as cardiomyocytes, with beneficial functional outcomes that are comparable to direct correction in undifferentiated iPSCs.

The main advantage of systemic application of Cas9-gRNA components using highly myotropic vectors such as AAV9 relies on the widespread and permanent correction of diseased muscle tissues including the heart.

### Example 2

In a further set of experiments, the AAV9-Cas9-gRNA was directly applied to heart vessels. Here, either the coronary artery was injected through an over-the-wire balloon during inflation of the balloon (e.g. blockade of the blood flow), allowing for prolongation of contact time and more efficacious virus transduction. Or a retrograde approach was chosen, where a Swan-Ganz catheter is inserted in the coronary vein accompanying the coronary artery. After balloon inflation selectively in the vein, the blood flow is reversed by gentle increase of blood pressure and the virus solution is injected over 5 min. In each case, the accompanying vessel is occluded by balloon inflation simultaneously, in order to increase contact time and to maximize efficacy (cf. FIG. 15)

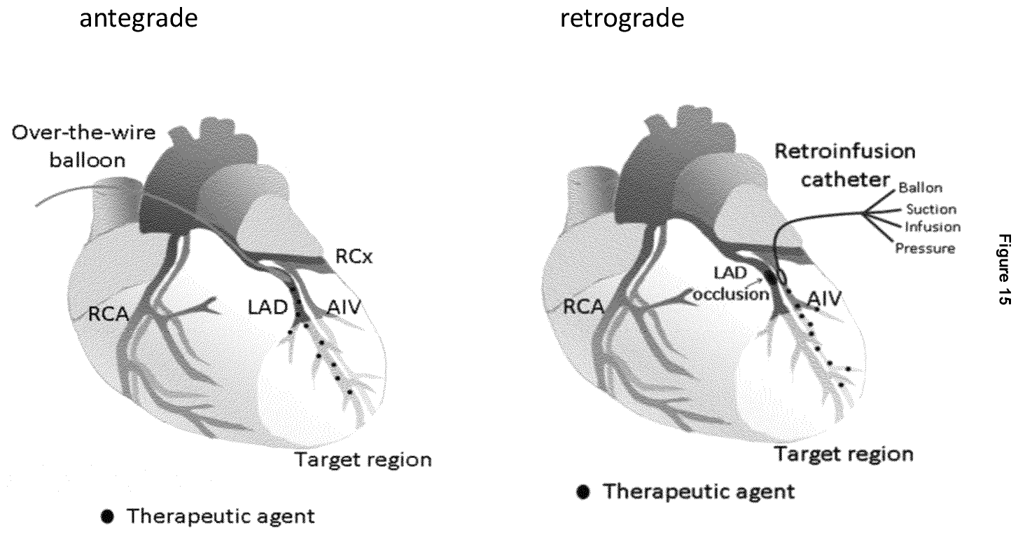

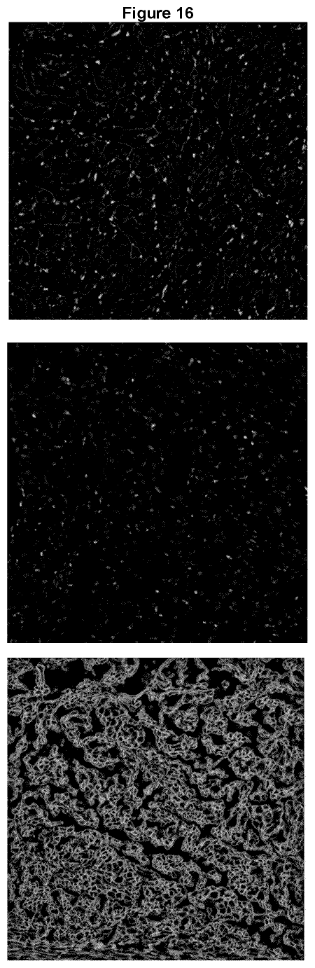

As a result, dystrophin expression, assessed by histology, is increased above the levels of systemic infusion 4 weeks after application of the same amount of the same virus agent (G2-AAV9-Cas9-gE51, as a 2-vector intein system) (cf. FIG. 16).

Thus, regional vascular gene editing therapy using AAV9 as vector system may further enhance the transduction efficacy in the heart.

### REFERENCES

1. Klymiuk, N., et al. Dystrophin-deficient pigs provide new insights into the hierarchy of physiological derangements of dystrophic muscle. Human Molecular Genetics 22, 4368-4382 (2013).
2. Truong, D. J., et al. Development of an intein-mediated split-Cas9 system for gene therapy. Nucleic Acids Res 43, 6450-6458 (2015).
3. Aartsma-Rus, A., Van Deutekom, J. C., Fokkema, I. F., Van Ommen, G. J. & Den Dunnen, J. T. Entries in the Leiden Duchenne muscular dystrophy mutation database: an overview of mutation types and paradoxical cases that confirm the reading-frame rule. Muscle Nerve 34, 135-144 (2006).
4. White, S., et al. Comprehensive detection of genomic duplications and deletions in the DMD gene, by use of multiplex amplifiable probe hybridization. Am J Hum Genet 71, 365-374 (2002).
5. Moser, H. Duchenne muscular dystrophy: pathogenetic aspects and genetic prevention. Human genetics 66, 17-40 (1984).
6. Sharp, P. S., Bye-a-Jee, H. & Wells, D. J. Physiological characterization of muscle strength with variable levels of dystrophin restoration in mdx mice following local antisense therapy. Mol Ther 19, 165-171 (2011).
7. van Deutekom, J. C., et al. Local Dystrophin Restoration with Antisense Oligonucleotide PRO051. New England Journal of Medicine 357, 2677-2686 (2007).
8. Goemans, N. M., et al. Systemic Administration of PRO051 in Duchenne's Muscular Dystrophy. New England Journal of Medicine 364, 1513-1522 (2011).
9. Verhaart, I. E., et al. The Dynamics of Compound, Transcript, and Protein Effects After Treatment With 2OMePS Antisense Oligonucleotides in mdx Mice. Molecular therapy. Nucleic acids 3, e148 (2014).
10. Bengtsson, N. E., et al. Muscle-specific CRISPR/Cas9 dystrophin gene editing ameliorates pathophysiology in a mouse model for Duchenne muscular dystrophy. Nat Commun 8, 14454 (2017).
11. E L Refaey, M., et al. In Vivo Genome Editing Restores Dystrophin Expression and Cardiac Function in Dystrophic Mice. Circulation Research (2017).
12. Long, C., et al. Postnatal genome editing partially restores dystrophin expression in a mouse model of muscular dystrophy. Science 351, 400-403 (2016).
13. Nelson, C. E., et al. In vivo genome editing improves muscle function in a mouse model of Duchenne muscular dystrophy. Science 351, 403-407 (2016).
14. Tabebordbar, M., et al. In vivo gene editing in dystrophic mouse muscle and muscle stem cells. Science 351, 407-411 (2016).
15. Amoasii, L., et al. Gene editing restores dystrophin expression in a canine model of Duchenne muscular dystrophy. Science (2018).
16. Punnoose, A. R., et al. Cardiac Disease Burden and Risk of Mortality in Hospitalized Muscular Dystrophy Patients. Pediatric Cardiology 37, 1290-1296 (2016).
17. Feingold, B., et al. Management of Cardiac Involvement Associated With Neuromuscular Diseases: A Scientific Statement From the American Heart Association. Circulation 136, e200-e231 (2017).
18. Vetter, A., et al. Adenoviral vectors coated with PAMAM dendrimer conjugates allow CAR independent virus uptake and targeting to the EGF receptor. Mol Pharm 10, 606-618 (2013).
19. Zincarelli, C., Soltys, S., Rengo, G. & Rabinowitz, J. E. Analysis of AAV Serotypes 1-9 Mediated Gene Expression and Tropism in Mice After Systemic Injection. Mol Ther 16, 1073-1080 (2008).
20. Thajudeen, A., et al. Correlation of scar in cardiac MRI and high-resolution contact mapping of left ventricle in a chronic infarct model. Pacing and clinical electrophysiology: PACE 38, 663-674 (2015).
21. Fischer C, M. H., Fein E, Reiser E, Lu K, Seidel T, Schinner C, Schwarzmayr T, Schramm R, Tomasi R, Husse B, Cao-Ehlker X, Pohl U, Dendorfer A. Long-term functional and structural preservation of precision-cut human myocardium under continuous electromechanical stimulation in vitro. Nat Commun in press (2019).
22. Moretti, A., et al. Patient-specific induced pluripotent stem-cell models for long-QT syndrome. N Engl J Med 363, 1397-1409 (2010).
23. Bar, S., et al. A novel product of the Duchenne muscular dystrophy gene which greatly differs from the known isoforms in its structure and tissue distribution. Biochem. J. 272, 557-560 (1990).
24. Kawaguchi, T., et al. Detection of Dystrophin Dp71 in Human Skeletal Muscle Using an Automated Capillary Western Assay System. Int. J. Mol. Sci. 19(2018).
25. Hinderer, C., et al. Severe toxicity in nonhuman primates and piglets following high-dose intravenous administration of an AAV vector expressing human SMN. Hum Gene Ther (2018).
26. Pleger, S. T., et al. Cardiac AAV9-S100A1 gene therapy rescues post-ischemic heart failure in a preclinical large animal model. Sci Transl. Med 3, 92ra64 (2011).
27. Walter, M. C. & Reilich, P. Recent developments in Duchenne muscular dystrophy: facts and numbers. 8, 681-685 (2017).
28. Aartsma-Rus, A., et al. Theoretic applicability of antisense-mediated exon skipping for Duchenne muscular dystrophy mutations. Human mutation 30, 293-299 (2009).
29. Wagner, D. L., et al. High prevalence of Streptococcus pyogenes Cas9-reactive T cells within the adult human population. Nature Medicine (2018).
30. Chew, W. L., et al. A multifunctional AAV-CRISPR-Cas9 and its host response. Nature Methods 13, 868 (2016).
31. Zhu, P., et al. CRISPR/Cas9-Mediated Genome Editing Corrects Dystrophin Mutation in Skeletal Muscle Stem Cells in a Mouse Model of Muscle Dystrophy. Molecular therapy. Nucleic acids 7, 31-41 (2017).
32. Klymiuk, N., et al. Dystrophin-deficient pigs provide new insights into the hierarchy of physiological derangements of dystrophic muscle. Human Molecular Genetics 22, 4368-4382 (2013).
33. Richter, A., et al. Potential of primary kidney cells for somatic cell nuclear transfer mediated transgenesis in pig. BMC biotechnology 12, 84 (2012).
34. Klymiuk, N., et al. Sequential targeting of CFTR by BAC vectors generates a novel pig model of cystic fibrosis. Journal of molecular medicine (Berlin, Germany) 90, 597-608 (2012).
35. Kurome, M., Kessler, B., Wuensch, A., Nagashima, H. & Wolf, E. Nuclear transfer and transgenesis in the pig. Methods Mol Biol 1222, 37-59 (2015).
36. Concordet, J. P. & Haeussler, M. CRISPOR: intuitive guide selection for CRISPR/Cas9 genome editing experiments and screens. Nucleic acids research 46, W242-W245 (2018).
37. Truong, D. J., et al. Development of an intein-mediated split-Cas9 system for gene therapy. Nucleic acids research 43, 6450-6458 (2015).
38. Kupatt, C., et al. Cotransfection of Vascular Endothelial Growth Factor-A and Platelet-Derived Growth Factor-B Via Recombinant Adeno-Associated Virus Resolves Chronic Ischemic Malperfusion: Role of Vessel Maturation. Journal of the American College of Cardiology 56, 414-422 (2010).
39. Kupatt, C., et al. Endothelial nitric oxide synthase overexpression provides a functionally relevant angiogenic switch in hibernating pig myocardium. Journal of the American College of Cardiology 49, 1575-1584 (2007).
40. Hinkel, R., et al. Diabetes Mellitus-Induced Microvascular Destabilization in the Myocardium. J Am Coll Cardiol 69, 131-143 (2017).
41. Pathik, B., et al. New Insights Into an Old Arrhythmia: High-Resolution Mapping Demonstrates Conduction and Substrate Variability in Right Atrial Macro-Re-Entrant Tachycardia. JACC: Clinical Electrophysiology 3, 971-986 (2017).
42. Childers, M. K., Grange, R. W. & Kornegay, J. N. In vivo canine muscle function assay. Journal of visualized experiments: JoVE (2011).
43. Gramlich, M., et al. Antisense-mediated exon skipping: a therapeutic strategy for titin-based dilated cardiomyopathy. EMBO molecular medicine (2015).
44. Lian, X., et al. Directed cardiomyocyte differentiation from human pluripotent stem cells by modulating Wnt/beta-catenin signaling under fully defined conditions. Nature protocols 8, 162-175 (2013).
45. Dorn, T., et al. Interplay of cell-cell contacts and RhoA/MRTF-A signaling regulates cardiomyocyte identity. EMBO J 37(2018).
46. Fischer, B., et al. A complete workflow for the differentiation and the dissociation of hiPSC-derived cardiospheres. Stem cell research 32, 65-72 (2018).
47. Van der Auwera, G. A., et al. From FastQ data to high confidence variant calls: the Genome Analysis Toolkit best practices pipeline. Curr Protoc Bioinformatics 43, 11.10.11-33 (2013).

1. A method of treating a disease in a subject in need thereof, the method comprising administering a vector system to said subject, the vector system comprising
(a) a first vector comprising a nucleic acid sequence encoding:
(i) a first fragment of an endonuclease,
(ii) a first fragment of an intein, and
(ii) a first guide RNA (gRNA); and
(b) a second vector comprising a nucleic acid sequence encoding:
(i) a second fragment of the endonuclease,
(ii) a second fragment of the intein, and
(ii) a second guide RNA (gRNA);
wherein the first gRNA binds to a region, which is located 5′ to a sequence of interest comprised in a nucleic acid sequence in the genome, optionally DNA, of a target cell,
wherein the second gRNA binds to a region located 3′ to the sequence of interest comprised in the nucleic acid sequence in the genome, optionally DNA, of a target cell;
wherein the first fragment and the second fragment of the intein are capable of associating into a functional intein,
wherein the functional intein is capable of ligating the first and the second fragment of the endonuclease to form a functional endonuclease;
wherein the functional endonuclease is capable of excising the sequence of interest.

2. The vector system for use of any of the preceding claims, wherein the endonuclease is Cas9, optionally Streptococcus pyogenes Cas9 (SpCas9), further optionally the Cas9 comprises an amino acid sequence as set forth in SEQ ID NO: 1 or an amino acid sequence having at least 60%, 70%, 80%, 85%, 90%, 95%, 98%, 99% or 100% sequence identity to a sequence as shown in SEQ ID 1.

3. The method of claim 1, wherein i) the first fragment of the endonuclease comprises an amino acid sequence selected from the group consisting of SEQ ID NOs: 2 and 3 or an amino acid sequence having at least 60%, 70%, 80%, 85%, 90%, 95%, 98%, 99% or 100% sequence identity to a sequence selected from the group consisting of SEQ ID NOs: 2 and 3, ii) the second fragment of the endonuclease comprises an amino acid sequence selected from the group consisting of SEQ ID NOs: 4 and 5 or an amino acid sequence having at least 60%, 70%, 80%, 85%, 90%, 95%, 98%, 99% or 100% sequence identity to a sequence selected from the group consisting of SEQ ID NOs: 4 and 5, or both i) and ii).

4. (canceled)

5. The method of claim 1, wherein the intein is selected from the group consisting of i) Npu of SEQ ID 6 or an amino acid sequence having at least 60%, 70%, 80%, 85%, 90%, 95%, 98%, 99% or 100% sequence identity to a sequence as shown in SEQ ID 6, ii) NrdJ-1 of SEQ ID 7 or an amino acid sequence having at least 60%, 70%, 80%, 85%, 90%, 95%, 98%, 99% or 100% sequence identity to a sequence as shown in SEQ ID 7 and a) gp-41 of SEQ ID 8 or an amino acid sequence having at least 60%, 70%, 80%, 85%, 90%, 95%, 98%, 99% or 100% sequence identity to a sequence as shown in SEQ ID 8.

6. The method of claim 1, wherein i) the first fragment of the intein comprises an amino acid sequence selected from the group consisting of SEQ ID NOs: 9-11 or an amino acid sequence having at least 60%, 70%, 80%, 85%, 90%, 95%, 98%, 99% or 100% sequence identity to a sequence selected from the group consisting of SEQ ID NOs: 9-11, ii) wherein the second fragment of the intein comprises an amino acid sequence selected from the group consisting of SEQ ID NOs: 12-14 or an amino acid sequence having at least 60%, 70%, 80%, 85%, 90%, 95%, 98%, 99% or 100% sequence identity to a sequence selected from the group consisting of SEQ ID NOs: 12-14, or both i) and ii).

7. (canceled)

8. The method of claim 1, wherein the first and/or the second vector is a viral vector, optionally wherein the viral vector is an adeno-associated virus (AAV) or lentivirus.

9. (canceled)

10. (canceled)

11. The method of claim 8, wherein the AAV is selected from the group consisting of AAV1, AAV2, AAV3, AAV4, AAV5, AAV6, AAV7, AAV8, AAV9, AAV10, and AAV11, or any combination thereof.

12. (canceled)

13. The method of claim 1, wherein the viral vector is coated with a dendrimer, optionally wherein the dendrimer is a PAMAM (poly(amidoamine)), further optionally wherein the dendrimer is a 2^{nd} generation PAMAM.

14. (canceled)

15. (canceled)

16. The method of claim 1, wherein the nucleic acid of the first and/or the second vector further comprises:
(iv) a nuclear localization signal, optionally comprising a sequence selected from the list group consisting of SEQ ID NOs: 15-20 or an amino acid sequence having at least 60%, 70%, 80%, 85%, 90%, 95%, 98%, 99% or 100% sequence identity to a sequence selected from the group consisting of SEQ ID NOs: 15-20.

17. The method of claim 1, wherein the first fragment of the nuclease and/or the second fragment of the intein and the nucleic acid(s) encoding the first and/or the second gRNA are operatively coupled to a promoter, wherein the promoter(s) optionally is/are inducible, optionally wherein i) the promoter that is operatively coupled to the first fragment of the nuclease and/or the second fragment of the intein is selected from the group consisting of CBH, B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, Mb promoter, NphsI promoter, SP-B promoter, SYN1 promoter and WASP promoter; ii) wherein the promotor that is operatively coupled to the first and/or the second gRNA is an RNA polymerase III promoter, further optionally selected from the group consisting of U6, H1 and 7SK; or both i) and ii).

18. (canceled)

19. (canceled)

20. The method of claim 1, wherein the method further comprises one or more of the following:
administering to the subject the first vector;
administering to the subject the second vector;
excising the sequence of interest.

21. (canceled)

22. The method of claim 1, wherein the first and the second vector are administered to the patient simultaneously or sequentially, optionally sequentially with a time delay of at least 5 minutes, at least 10 minutes, at least 15 minutes, at least 30 minutes, of at least 1 hour, at least 2 hours, at least 3 hours, at least 4 hours, at least 8 hours, at least 12 hours, at least 24 hours, at least 48 hours, at least 72 hours, at least 1 week or at least 2 weeks.

23. (canceled)

24. The method of claim 1, wherein the subject is a mammal, optionally a human or a pig.

25. The method of claim 1, wherein the first and the second vectors are administered systemically, enterally, parenterally, intravenously, intra-arterially, topically, intraperitoneally, intramuscularly, intradermally, intrathecally, intravitreally, subcutaneously, transdermally and/or transmucosally.

26. The method of claim 1, wherein the disease is selected from the group consisting of Duchenne muscular dystrophy, hereditary myopathy with early respiratory failure, early-onset myopathy with fatal cardiomyopathy, core myopathy with heart disease, centronuclear myopathy, limb-girdle muscular dystrophy type 2J, familial dilated cardiomyopathy 9, hypertrophic cardiomyopathy and tibial muscular dystrophy, optionally Duchenne muscular dystrophy, further optionally Duchenne muscular dystrophy characterized by a deletion of exon 52 of the dystrophin gene.

27. (canceled)

28. The method of claim 1, wherein i) the nucleic acid sequence of interest is exon 51 of the dystrophin gene, optionally the exon 51 comprises a sequence of 23 or 24 or a nucleic acid sequence having at least 60%, 70%, 80%, 85%, 90%, 95%, 98%, 99% or 100% sequence identity to a sequence as shown in SEQ ID 23 or 24, ii) wherein the first gRNA comprises a nucleic acid sequence as set forth in any of SEQ ID NOs: 25 or 26 or a nucleic acid sequence having at least 60%, 70%, 80%, 85%, 90%, 95%, 98%, 99% or 100% sequence identity to a sequence as shown in SEQ ID NOs: 25 or 26 and/or the second gRNA comprises a nucleic acid sequence as set forth in any of SEQ ID NOs: 27 or 28 or a nucleic acid sequence having at least 60%, 70%, 80%, 85%, 90%, 95%, 98%, 99% or 100% sequence identity to a sequence as shown in SEQ ID NOs: 27 or 28, or both i) and ii).

29. (canceled)

30. (canceled)

31. A vector system comprising
(a) a first vector comprising a nucleic acid sequence encoding:
(i) a first fragment of an endonuclease,
(ii) a first fragment of an intein, and
(ii) a first guide RNA (gRNA); and
(b) a second vector comprising a nucleic acid sequence encoding:
(i) a second fragment of the endonuclease,
(ii) a second fragment of the intein, and
(ii) a second guide RNA (gRNA);
wherein the first gRNA binds to a region, which is located 5′ to a sequence of interest comprised in a nucleic acid sequence in the genome, optionally DNA, of a target cell,
wherein the second gRNA binds to a region located 3′ to the sequence of interest comprised in the nucleic acid sequence in the genome, optionally DNA, of a target cell;
wherein the first fragment and the second fragment of the intein are capable of associating into a functional intein,
wherein the functional intein is capable of ligating the first and the second fragment of the endonuclease to form a functional endonuclease;
wherein the functional endonuclease is capable of excising the sequence of interest.

32. The first vector, the second vector, or both the first and the second vector, as defined in claim 31.

33. (canceled)

34. (canceled)

35. A pharmaceutical composition comprising the vector system of claim 31.

36. A method for excising a sequence of interest from the genome, optionally DNA, of a subject, the method comprising administering the vector system of claim 31 thereby excising the sequence of interest from the genome, optionally DNA, of a subject.
